SEQUENCE LISTING (8 sequences)

SEQ ID NO: 1 - DNA, 21 bp, synthetic construct
  source 1..21: mol_type = other DNA; note = shNT
gagtgccactttccgaataaa

SEQ ID NO: 2 - DNA, 21 bp, synthetic construct
  source 1..21: mol_type = other DNA; note = shAbca1
gcgcgatagcgctaataattt

SEQ ID NO: 3 - DNA, 687 bp, synthetic construct
  source 1..687: mol_type = other DNA; note = gfaABC1D
gatctaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgca

SEQ ID NO: 4 - DNA, 25 bp, synthetic construct
  source 1..25: mol_type = other DNA; note = sgLuc2
caccgttggcgctcaacttttacga

SEQ ID NO: 5 - DNA, 25 bp, synthetic construct
  source 1..25: mol_type = other DNA; note = SgAbca1 (#1)
caccggagagtcactcacccggaca

SEQ ID NO: 6 - DNA, 25 bp, synthetic construct
  source 1..25: mol_type = other DNA; note = sgAbca1 (#2)
caccgttggcgctcaacttttacga

SEQ ID NO: 7 - DNA, 18 bp, synthetic construct
  source 1..18: mol_type = other DNA; note = sgRNA sequencing primer
tacgtgacgtagaaagta

SEQ ID NO: 8 - DNA, 986 bp, synthetic construct
  source 1..986: mol_type = other DNA; note = ABCA1 promoter
catgttcctctcgggtcctctgagggacctggggagctcaggctgggaatctccaaggcagtaggtcgcctatcaaaaatcaaagtccaggtttgtggggggaaaacaaaagcagcccattacccagaggactgtccgccttcccctcaccccagcctaggcctttgaaaggaaacaaaagacaagacaaaatgattggcgtcctgagggagattcagcctagagctctctctcccccaatccctccctccggctgaggaaactaacaaaggaaaaaaaaattgcggaaagcaggatttagaggaagcaaattccactggtgcccttggctgccgggaacgtggactagagagtctgcggcgcagccccgagcccagcgcttcccgcgcgtcttaggccggcgggcccgggcgggggaaggggacgcagaccgcggaccctaagacacctgctgtaccctccacccccaccccacccacctccccccaactccctagatgtgtcgtgggcggctgaacgtcgcccgtttaaggggcgggccccggctccacgtgctttctgctgagtgactgaactacataaacagaggccgggaacggggcggggaggagggagagcacaggctttgaccgatagtaacctctgcgctcggtgcagccgaatctataaaaggaactagtcccggcaaaaaccccgtaattgcgagcgagagtgagtggggccgggacccgcagagccgagccgacccttctctcccgggctgcggcagggcagggcggggagctccgcgcaccaacagccggttctcagggcgctttgctccttgttttttccccggttctgttttctccccttctccggaaggcttgtcaaggggtaggagaaagagacgcaaacacaaaagtggaaaacaggtaagaggctctccagtgacttacttgggcgttattgttttgtttcgaggccaaggaggcttcgggaagtga

(19) United States
(12) Patent Application Publication
(10) Pub. No.: US 2025/0082766 A1
(43) Pub. Date: Mar. 13, 2025

(54) TREATMENT OF BRAIN TUMORS BY TARGETING THE CHOLESTEROL PATHWAY IN ASTROCYES

(71) Applicant: Ramot at Tel-Aviv University Ltd., Tel-Aviv (IL)

(72) Inventor: Lior MAYO, Tel-Aviv (IL)

(73) Assignee: Ramot at Tel-Aviv University Ltd., Tel-Aviv (IL)

(21) Appl. No.: 18/897,070

(22) Filed: Sep. 26, 2024

Related U.S. Application Data
(63) Continuation of application No. PCT/IL2023/050337, filed on Mar. 30, 2023.
(60) Provisional application No. 63/325,655, filed on Mar. 31, 2022.

(51) Int. Cl. A61K 47/68; A61P 35/00; C12N 15/113

(52) U.S. Cl. CPC A61K 47/6803; A61P 35/00; C12N 15/113; C12N 2310/14; C12N 2310/531

(57) ABSTRACT

A method of treating a brain tumor in a subject in need thereof is disclosed. The method comprising administering to the subject a therapeutically effective amount of an agent capable of downregulating activity or expression of a component of the lipid synthesis and/or transportation pathways in an astrocyte in the tumor microenvironment and/or a therapeutically effective amount of a molecule which is associated with lipid uptake by the tumor cells or immune cells in the tumor microenvironment, or a polynucleotide encoding same.

### RELATED APPLICATIONS

This application is a Continuation of PCT Patent Application No. PCT/IL2023/050337 having International filing date of Mar. 30, 2023, which claims the benefit of priority under 35 USC § 119(e) of U.S. Provisional Patent Application No. 63/325,655, filed on Mar. 31, 2022.

PCT Patent Application No. PCT/IL2023/050337 is also related to co-filed PCT Patent Application No. PCT/IL2023/050338, entitled “TARGETING IMMUNE INFILTRATION TO THE CENTRAL NERVOUS SYSTEM (CNS)” (Attorney Docket No. 95282).

The contents of the above applications are all incorporated by reference as if fully set forth herein in their entirety.

### SEQUENCE LISTING STATEMENT

The XML file, entitled 100921.xml, created on Sep. 23, 2024, comprising 10,777 bytes, submitted concurrently with the filing of this application is incorporated herein by reference.

### FIELD AND BACKGROUND OF THE INVENTION

The present invention, in some embodiments thereof, relates to targeting the lipid synthesis in astrocytes and, more particularly, but not exclusively, to the use of same for the treatment of brain tumors.

Cancers develop in complex tissue-dependent environments, on which they rely for sustained growth, invasion and metastasis. The tumor microenvironment (TME) is associated with response to therapy and with regulation of the immune response to the tumors. Unlike the tumor cells, non-neoplastic cells in the tumor microenvironment (TME) are genetically stable and thus represent an attractive therapeutic target, with a lower risk of resistance and tumor recurrence. Accumulating data suggest that modulation of the bioenergetic, metabolic, or immunological properties of the TME can regulate tumor progression in the central nervous system (CNS). However, the mechanisms that shape the immunometabolic landscape of the TME, and thus control brain cancer pathogenesis, are not well understood.

Glioblastoma Multiforme (GBM) is the most common primary malignant brain tumor and carries an abysmal 5-year survival rate of just 5.6%, a statistic that has shown little change over decades. Despite significant advances in the understanding of this disease, the translation to improved treatment has been quite disappointing. Thus, neurosurgery, radiation, and cytotoxic chemotherapy (mainly Temozolomide; TMZ) remain the mainstays of therapy along with agents which inhibit formation of blood vessels (angiogenesis) and peritumoral edema, although, immune checkpoint therapy has recently been suggested as a potentially efficacious approach.

Glioblastoma is notoriously genetically unstable, thereby limiting the options for targeted therapies. The tumor microenvironment supports tumor metabolism and strengthens the resistance of glioblastoma to radiation and chemotherapy and the low immunogenicity of glioblastoma hinders a strong immunological response. GBM tumors are considered to have a high apoptotic threshold, which might contribute to their resistance to cytotoxic therapy. Moreover, the blood brain barrier (BBB) prevents many systemically administered chemotherapeutics from reaching sufficient concentrations in the brain without serious adverse effects.

The ability of a cell to undergo intrinsic apoptosis or mitochondrial programmed cell death is governed by the interactions of the BCL-2 family of proteins. Higher mitochondrial priming, meaning a higher possibility to begin apoptosis, was shown to increase chemosensitivity and response to therapy in different types of cancers (Vo, T.-T. et al. 2012. Cell 151, 344-355).

In many cancers, the tumor cell metabolism adapts during oncogenic transformation to ideally support their growing metabolic and energetic demands and can raise their apoptotic threshold. Specifically, brain tumors display a high cholesterol content, which is thought to support tumor growth and the viability and activity of cells in the TME. Glioma cells exhibit an accumulation of cholesterol, specifically in their mitochondria. Cholesterol presence in membranes, associated with ABCA1 activity, decreases their fluidity, which in turn inhibits mitochondrial permeability transition and release of pro-apoptotic signaling. Moreover, Cholesterol levels are also known to regulate gene expression (e.g., via LXR, Hippo pathway/p53, or ERK signaling), which can regulate the expression of pro- or anti-apoptotic BCL-2 family genes.

Targeted disruption of cholesterol metabolism was shown to be beneficial in adult and childhood brain tumors [Phillips, R. E., et al., Proc Natl Acad Sci USA (2019) 116: 7957-7962; Villa, G. R., et al., Cancer Cell (2016) 30: 683-693]. Specifically, the potential to target cholesterol metabolism as a new strategy for treating glioblastomas was discussed in Ahmed et al., Cancers (Basel) (2019) 11(2): 146.

Astrocytes are the most abundant cells in the CNS. They perform essential functions during development and homeostasis, such as participating in the maintenance of the blood-brain barrier (BBB), storing and distributing energetic substrates to neurons, and supporting the development of neural cells and synaptogenesis. Astrocytes can also control CNS inflammation and neurodegeneration through multiple mechanisms, including neurotoxicity, modulation of microglial activities, recruitment of inflammatory cells into the CNS, and even via their metabolic cascade. Tumor-associated astrocytes (TAAs) were recently suggested to participate in shaping the TME of primary and secondary brain tumors [Henrik Heiland, D., et al. Nat Commun (2019) 10: 2541; Priego, N., et al. Nat Med (2018) 24: 1024-1035; Chen, Q., et al. Nature (2016) 533: 493-498]. However, the role of reactive astrocytes in GBM pathogenicity is poorly understood.

Ming-Xue Piao et al., (2015. Int. J. Clin. Exp. Pathol. 2015; 8(3): 2787-2794; “PCSK9 regulates apoptosis in human neuroglioma u251 cells via mitochondrial signaling pathways”) shows that PCSK9 overexpression resulted in increased cell proliferation of U251 cells, and suggest that a PCSK9 inhibitor may be useful in therapies directed against neuroglioma and possibly other types of cancer.

Additional background art includes: Pirmoradi L. et al., J Investig Med (2019) 67(4):715-719.

### SUMMARY OF THE INVENTION

According to an aspect of some embodiments of the present invention there is provided a method of treating a brain tumor in a subject in need thereof, the method comprising administering to the subject a therapeutically effective amount of an agent capable of specifically downregulating activity or expression of a component of the lipid synthesis and/or transportation pathways in an astrocyte in the tumor microenvironment, wherein the agent is specific to the astrocyte in the tumor microenvironment and not to a cancerous cell of the brain tumor, thereby treating the brain tumor in the subject.

According to an aspect of some embodiments of the present invention there is provided a therapeutically effective amount of an agent capable of specifically downregulating the activity or expression of a component of the lipid synthesis and/or transportation pathways in an astrocyte in the tumor microenvironment, wherein the agent is specific to the astrocyte in the tumor microenvironment and not to a cancerous cell of the brain tumor for use in treating a brain tumor in a subject in need thereof.

According to an aspect of some embodiments of the present invention there is provided a method of treating a brain tumor in a subject in need thereof, the method comprising administering to the subject a therapeutically effective amount of a molecule which is associated with lipid uptake by the tumor cells or immune cells in the tumor microenvironment, or a polynucleotide encoding same, thereby treating the brain tumor in the subject.

According to an aspect of some embodiments of the present invention there is provided a therapeutically effective amount of a molecule which is associated with lipid uptake by the tumor cells or immune cells in the tumor microenvironment, or a polynucleotide encoding same, for use in treating a brain tumor in a subject in need thereof.

According to an aspect of some embodiments of the present invention there is provided a chimeric polynucleotide comprising a nucleic acid sequence encoding an expression product capable of downregulating an activity or expression of a component of the lipid synthesis and/or transportation pathways, and another heterologous nucleic acid sequence comprising a cis acting regulatory element specifically active in a reactive astrocyte of the microenvironment of the tumor but not in a non-reactive astrocyte or a cancerous cell of a brain tumor.

According to an aspect of some embodiments of the present invention there is provided a composition of matter comprising the chimeric polynucleotide of some embodiments of the invention, comprising a particle encapsulating the chimeric polynucleotide.

According to an aspect of some embodiments of the present invention there is provided an article of manufacture comprising a small molecule capable of downregulating an activity or expression of a component of the lipid synthesis and/or transportation pathways, the small molecule being conjugated to an antibody or fragment thereof capable of binding to a reactive astrocyte in the tumor microenvironment and not to a non-reactive astrocyte or a cancerous cell of the brain tumor.

According to an aspect of some embodiments of the present invention there is provided a monocyte expressing a heterologous PCSK9 mRNA (messenger RNA) or protein.

According to an aspect of some embodiments of the present invention there is provided an astrocyte expressing a heterologous PCSK9 mRNA or protein.

According to some embodiments of the invention, the astrocyte comprises a reactive astrocyte.

According to some embodiments of the invention, the agent is specific to a reactive astrocyte and not to a cancerous cell of the brain tumor.

According to some embodiments of the invention, the lipid is selected from the group consisting of a cholesterol, a cholesteryl ester (CE), a triglyceride and a sphingolipid.

According to some embodiments of the invention, the agent is an efflux inhibitor which inhibits release of a lipid from the astrocyte in the tumor microenvironment.

According to some embodiments of the invention, the lipid is cholesterol.

According to some embodiments of the invention, the efflux inhibitor is an inhibitor of the ATP-binding cassette transporter A1 (ABCA1).

According to some embodiments of the invention, the agent is a small molecule.

According to some embodiments of the invention, the small molecule is Probucol or Glyburide, or an analogue thereof.

According to some embodiments of the invention, the component of the lipid synthesis or transportation pathway is a molecule associated with the de-novo cholesterol synthesis pathway.

According to some embodiments of the invention, the component of the lipid synthesis or transportation pathway is a molecule associated with cholesterol catabolism to oxysterols.

According to some embodiments of the invention, the component of the lipid synthesis or transportation pathway is a molecule associated with liver-X-receptors (LXRs).

According to some embodiments of the invention, the component of the lipid synthesis or transportation pathway is a molecule associated with oxysterols catabolism.

According to some embodiments of the invention, the component of the lipid synthesis or transportation pathway is a molecule associated with cholesterol efflux.

According to some embodiments of the invention, the component of the lipid synthesis or transportation pathway is a molecule associated with cholesterol uptake.

According to some embodiments of the invention, the component of the lipid synthesis or transportation pathway is a molecule associated with the induction of Mylip.

According to some embodiments of the invention, the component of the lipid synthesis or transportation pathway is an ATP-binding cassette transporter A1 (ABCA1).

According to some embodiments of the invention, the small molecule is selected from the group consisting of a Probucol, Glyburide, a LDLR antisense/decoy molecule, a Menin inhibitor, a Statin, AY-9944, D-003, Avasimibe, Nystatin, Ezetimibe, Fenofibrate, 2-Hydroxypropyl-β-cyclodextrin, Omega-3-acid ethyl esters and an analogue thereof.

According to some embodiments of the invention, the agent is a DNA editing molecule.

According to some embodiments of the invention, the DNA editing molecule is selected from the group consisting of a meganuclease, a zinc finger nuclease (ZFN), a transcription-activator like effector nuclease (TALEN) and CRISPR/Cas system.

According to some embodiments of the invention, the agent is a RNA silencing molecule.

According to some embodiments of the invention, the RNA silencing molecule is selected from the group consisting of an antisense oligonucleotide (ASO), a small interference RNA (siRNA), a microRNA (miRNA), a short hairpin RNA (shRNA), a DNAzyme and a ribozyme.

According to some embodiments of the invention, the agent is comprised in a nucleic acid construct under the transcriptional control of a cis acting regulatory element specifically active in the astrocyte.

According to some embodiments of the invention, the cis acting regulatory element is a promoter.

According to some embodiments of the invention, the cis acting regulatory element is an astrocyte-specific promoter.

According to some embodiments of the invention, the astrocyte-specific promoter is a glial fibrillary acidic protein (GFAP) promoter.

According to some embodiments of the invention, the glial fibrillary acidic protein (GFAP) promoter comprises SEQ ID NO: 3.

According to some embodiments of the invention, the nucleic acid construct is encapsulated in a particle.

According to some embodiments of the invention, the particle is an Adeno-associated virus (AAV) particle.

According to some embodiments of the invention, the small molecule is conjugated to an antibody or fragment thereof capable of binding the astrocyte.

According to some embodiments of the invention, the antibody is a T cell receptor-like antibody.

According to some embodiments of the invention, the antibody is capable of binding a reactive astrocyte MHC-I complex.

According to some embodiments of the invention, the agent is conjugated directly or indirectly to a targeting moiety capable of binding the astrocyte of the tumor microenvironment and not to a non-reactive astrocyte or a cancerous cell of the brain tumor.

According to some embodiments of the invention, the targeting moiety is an antibody, an aptamer, a peptide or a particle.

According to some embodiments of the invention, the method further comprising administering to the subject a therapeutically effective amount of a molecule which is associated with lipid uptake by the tumor cells or immune cells in the tumor microenvironment.

According to some embodiments of the invention, the molecule which is associated with lipid uptake by the tumor cells or immune cells in the tumor microenvironment is Proprotein convertase subtilisin/kexin type 9 (PCSK9).

According to some embodiments of the invention, the molecule or the polynucleotide is comprised in or associated with a particle suitable for delivery into a brain of the subject.

According to some embodiments of the invention, the particle is acellular.

According to some embodiments of the invention, the particle is cellular.

According to some embodiments of the invention, the cellular particle is a cell.

According to some embodiments of the invention, the cell is a monocyte.

According to some embodiments of the invention, the cell is an astrocyte.

According to some embodiments of the invention, the acellular particle is an Adeno-associated virus (AAV) particle.

According to some embodiments of the invention, the method further comprising administering to the subject chemotherapy.

According to some embodiments of the invention, the chemotherapy is temozolomide (TMZ).

According to some embodiments of the invention, the method further comprising administering radiation therapy to the subject.

According to some embodiments of the invention, the method further comprising administering to the subject an anti VEGF antibody.

According to some embodiments of the invention, the brain tumor is selected from the group consisting of Acoustic neuroma, Astrocytoma, Choroid plexus carcinoma, Craniopharyngioma, Embryonal tumor, Ependymoma, Glioblastoma, Glioma, Medulloblastoma, Meningioma, Oligodendroglioma, Pediatric brain tumor, Pineoblastoma, Pituitary tumor and Brain metastasis. According to some embodiments of the invention, the brain tumor is Glioblastoma.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I illustrate that depletion of Tumor-associated astrocytes (TAAs) regress glioblastoma progression. (FIG. 1A) Representative immunofluorescence images of reactive TAAs stained for GFAP (cyan) crowning a glioblastoma (GBM) tumor (GFP⁺-GL261, white), and nuclei (DAPI, yellow). The right image is an expansion of the area marked by the white box. (n=3 biologically independent experiments, 3 mice per group). Scale bar, 1000 m; (FIGS. 1B-I) WT (wild type), or Gfap-TK GBM-bearing mice were treated daily with Ganciclovir (GCV, 25 mg/kg) from day 10 until the experimental endpoint as illustrated in FIG. 1B. (FIG. 1C) representative immunofluorescence images of reactive astrocyte depletion at the tumor margins (GFP⁺-GL261, white), as detected by GFAP staining (cyan). Scale bars, 500 m. Data are representative of three independent experiments with n=4 mice/group. (FIGS. 1D-G) Tumor size in GCV-treated WT or Gfap-TK GBM-bearing littermates. (FIGS. 1D-E) Representative images of GL261-derived bioluminescence from each group are shown in FIG. 1D and quantification of tumor size in FIG. 1E. Data are representative of five independent experiments with n=6 mice/group. (FIGS. 1F-G) Representative images from each group, 17 days after GL261 cell implantation, are shown in FIG. 1F with quantification of tumor size in FIG. 1G, tumor (GL261 cells in purple), and nuclei (DAPI; white). Scale bar, 1000 m. Data are representative of three independent experiments with n=5 mice/group. (FIG. 1H) Bodyweight assessment of mice from FIGS. 1D-E. (FIG. 1I) Kaplan-Meier curves assessing overall survival. Data are representative of three independent experiments with n=8 mice/group. Data in FIGS. 1D-H are shown as mean±s.e.m. P values were determined by two-way ANOVA (FIGS. 1D-H) or Log rank (Mantel-Cox) test (FIG. 1I). *P<0.05, **P<0.01, ***P<0.001, n.s. not significant.

FIGS. 2A-F illustrate a RiboTag analysis of Tumor associated-astrocytes revealing activation of immunoregulatory pathways and perturbation of metabolic circuits. (FIG. 2A) Illustration of the RiboTag workflow; (FIG. 2B) Enrichment of astrocyte-specific gene expression and de-enrichment of neuronal, oligodendroglial, and Tumor-associated macrophages (TAMs) gene expression, shown as the log fold change calculated between astrocyte RNAs immunoprecipitated by anti-HA antibody (anti Haemagglutinin antibody) versus brain total cell RNAs (including astrocytes) immunoprecipitated with control antibody; (FIG. 2C) Representative immunofluorescence images of GfapCRE:Rpl22HA mice demonstrating co-localization of ribosome-associated HA-Tag (yellow) with specific cell-lineage markers of astrocytes, TAMs, oligodendrocytes, or neurons cell-linage specific markers (GFAP, IBA1, MBP, or NeuN, respectively; blue). Co-localization (white) is identified by red arrowheads. Scale bars, m; (FIG. 2D) heatmap of differently expressed genes (at least two-fold, padj<0.01) of astrocytes derived from sham-injected or GBM-bearing brain hemisphere specimens; (FIG. 2E) RiboTag-isolated mRNA expression in astrocytes from GL261-bearing (GBM) or PBS-injected (Sham) Gfap^{Cre}:Rpl22^{HA} mice, 17 days after intracranial injection. Data are representative of three independent experiments with n=4 biologically independent samples, pool of 2 mice per sample; (FIG. 2F) Manhattan plot of gene ontology (GO) of upregulated in TAAs. Similar pathways are color-coded: Immune-regulation (green), metabolism (pink), proliferation (blue), Miscellaneous (orange). Highlighted GO are numbered and detailed in Table 1 (herein below). Data in FIGS. 2B and 2E are shown as mean±s.e.m. P values were determined by one-way ANOVA (FIG. 2B) or two-sided Student's t-tests (FIG. 2E). *P<0.05, **P<0.01, ***P<0.001.

FIGS. 3A-I illustrate that TAA depletion attenuates TAM recruitment. (FIG. 3A) Primary astrocytes stimulated with complete medium (Med) or GBM-CM from GL261 cells for 6 hours. Quantitative PCR (qPCR) analysis of Ccl2 expression normalized to Ppia (n=4 biologically independent experiments); (FIGS. 3B-C) Astrocytes were stimulated with complete medium or GBM-CM for 12 hours, extensively washed, and used to prepare astrocyte conditioned medium (ACM) or tumor cell-induced ACM (T-ACM), which was tested in an in vitro monocyte migration assay; (FIGS. 3D-G) Analysis of TAMs in the tumor microenvironment. Green Fluorescent Protein (GFP)⁺-GL261 glioma cells were intracranially injected into WT or Gfap-TK mice. Following tumor establishment (day 9 after tumor implantation), mice were treated daily with GCV (as in FIG. 1B); TAMs recruitment to the tumor was examined 17 days after tumor implantation. (FIGS. 3D-E) Representative immunofluorescence images TAMs stained for IBA-1 (white) in the tumor (GBM, green) microenvironment (n=2 biologically independent experiments, 4 mice per group). Scale bars, 100 m. (FIGS. 3F-G) Percentage of TAMs (CD11b⁺ CD45⁺) gated from GCV-treated GL261-bearing WT and Gfap-TK mice. Representative flow cytometry plots from each group are shown in FIG. 3F and quantification analyses are in FIG. 3G. (n=3 independent experiments, 6 mice per experiment). Data are shown as mean±s.e.m. P values were determined by one-way ANOVA, followed by Fisher's LSD post-hoc analysis (FIG. 3B) or two-sided Student's t-tests (FIGS. 3A, 3C, 3D). *P<0.05, **P<0.01, ***P<0.001. (FIGS. 3H-I) Heatmap of differently expressed chemokines (at least two-fold, Padj<0.01) (FIG. 3H) and Ccl2 mRNA expression (shown in FIG. 2E) in astrocytes derived from sham-injected or GBM-bearing mice, as described in FIG. 2E. Data are representative of three independent experiments with n=4 biologically independent samples. (FIG. 3I) scRNAseq analysis of chemokine expression intensity (color-coded), frequency (dot size), and Z-score in tumor-associated astrocytes from GBM patients (astrocyte cluster as in FIG. 8A). Expression levels (FIGS. 3H and 3I) are defined by color-coded expression as indicated (from blue to pink).

FIGS. 4A-K illustrate that TAA ablation attenuates TAM recruitment. (FIGS. 4A-D) Functional analysis of TAMs (CD11b⁺DC45⁺) isolated from GCV-treated WT and Gfap-TK GL261-implanted mice (as in FIGS. 3D-G) 17 days after tumor implantation. (FIG. 4A) Pathway enrichment analysis of differentially expressed genes (at least two-fold, padj<0.05) as detected by Nanostring (FIG. 10A). (FIGS. 4B-C) qPCR analysis of Arg1, Ahr, Stat3, Irf7, Gpnmb, Vegfa, Mmp14 and Cd274 expression in FACS-sorted TAMs; expression normalized to Ppia. Data are representative of 3 independent experiments (n=4 biologically independent samples, pool of 2 mice per sample). (FIG. 4D) PD-L1 expression in TAMs. Representative flow cytometry plots from each group are shown on the left and quantification analyses of the percentage of PD-LI⁺ TAMs and PD-L1 expression (geometric mean fluorescence intensity, gMFI) are on the right. Data are representative of 3 independent experiments (n=4 biologically independent samples, pool of 2 mice per sample). (FIGS. 4E-F) Mixed glia cultures were treated with mild Trypsin/EDTA (T/E) to remove the astrocyte monolayer leaving only the microglia attached to the plate, or were left un-treated. Cultures were then treated with GL261 conditioned media (GBM-CM) for 72 hours. Representative flow cytometry plots of microglial (gated as CD11b⁺ cells) PD-L1 expression from each group are shown on the left and quantification analyses of the percentage of PD-LI⁺ microglial cells are on the right (n=4 biologically independent experiments). (FIG. 4G) Mixed glia and MG cultures were prepared as in (FIG. 4E) and treated with GBM-CM for 24 hours. MG cultures were then isolated with mild T/E, and co-cultured with GFP⁺-GL261 cells for 48 hours. The viability of GFP-gated GL261 cells was then determined by Annexin-V assay. (FIG. 4H) Representative flow cytometry plots of GFP-gated GL261 cells from each group are shown on the left, and quantification analyses of cell death are on the right (n=4 biologically independent experiments). (FIG. 4I) microglial cultures were pre-treated with N-Nitro-L-Arginine Methyl Ester (L-NAME) (2 mM) before co-incubation with GL261 cells, and glioma viability was analyzed (n=3 biologically independent experiments). (FIG. 4J) qPCR analysis of Nos2 expression in Ribotag-isolated microglial cells (CD11b⁺CD45^{dim}) isolated from GCV-treated WT and Gfap-TK GBM-bearing mice, as in (FIG. 2E); expression normalized to Ppia. Data are representative of 3 independent experiments (n=3 biologically independent samples, pool of 2 mice per sample). (FIG. 4K) Mixed glia were treated with the indicated blocking antibodies or appropriate isotype controls (25 g/ml), and then activated with GBM-CM. Microglia were isolated as in (FIG. 4E), and microglial expression of Nos2 was determined by qRT relative to Ppia (n=5 biologically independent experiments). Data are shown as mean±s.e.m. P values were determined by two-sided Student's t-tests (FIGS. 4A-J) and one-way ANOVA, followed by Fisher's LSD post-hoc analysis (FIG. 4K) or *P<0.05, **P<0.01, ***P<0.001, ns—not significant.

FIGS. 5A-N illustrate that astrocyte-derived cholesterol supports glioma survival. (FIGS. 5A-D) Real-time changes in the ECAR (FIGS. 5A-B) and oxygen consumption rate (OCR) (FIGS. 5C-D) of GL261 glioma cells, cultured in media supplemented with full serum (FCS, fetal calf serum) or lipoprotein-deprived serum (LPDS, Lipoprotein-deficient serum) for 18 hours and measured using Seahorse. Oligo, oligomycin; FCCP, carbonyl cyanide4-(trifluoromethoxy) phenylhydrazone; R/A, rotenone plus antimycin A; 2-DG, 2-deoxy-d-glucose. Glycolysis, glycolytic capacity and glycolytic reserve were extracted from the ECAR reading, and basal respiration, adenosine triphosphate (ATP) production, Maximal respiration, and spare respiratory capacity were determined based on OCR. Data are representative of two independent experiments (n=6 technical replicates per experiment). (FIG. 5E) Percentage of cell death of GL261 and CT-2A glioma cells, or primary astrocytes, cultured in FCS or LPDS for 5 days; as determined by Annexin-V assay (n=5 independent experiments). (FIGS. 5F-G) GL261 or CT-2A glioma cells were cultured in FCS or LPDS-media and treated with PBS (Mock) or cholesterol (250 ng/ml) for 5 days. Representative flow cytometry plots of Annexin-V/DAPI staining from each group are shown in FIG. 5F and quantification analyses of cholesterol rescue are in FIG. 5G (n=5 biologically independent experiments). (FIGS. 5H-K) Representative images and quantification of LDLR expression (Immunoblot, FIGS. 5H-I) and cell death via FACS analysis of Annexin V/DAPI staining (FIGS. 5J-K) in response to endogenous LXR agonists (24-OHC) or Mock (DMSO) treatment in GL261 and CT-2A cells (n=3 biologically independent experiments). (FIGS. 5L-N) Analysis of LPDS-induced glioma cell death, in the presence of absence of primary astrocytes, by Annexin-V assay. (FIGS. 5L-M) Representative flow cytometry plots of murine GL261 and CT-2A glioma cells co-cultured for 5 days with primary mouse astrocytes are shown in FIG. 5L and quantification analyses in FIG. 5M (n=4 biologically independent experiments). (FIG. 5N) quantification analyses of human U87EGFRvIII (U87) glioma cells co-cultured with human primary astrocytes for 5 days (n=3 biologically independent experiments). Data are shown as mean±s.e.m. P values were determined by two-sided Student's t-tests (FIGS. 5C-G, 5L-N) or by Two-way ANOVA (FIGS. 5H-K). *P<0.05, **P<0.01, ***P<0.001.

FIGS. 6A-O illustrate that astrocytic expression of ABCA1 regulates glioma cholesterol levels and tumor progression. (FIG. 6A) Box plot analysis of the cancer genome atlas (TCGA) gene expression for ABCA1 in normal (n=207) or GBM patients (n=163). n represents the number of patients per group. (FIG. 6B) Heat map overly of the scRNAseq gene expression intensity of ABCA1, and ABCG1 in TAAs from GBM patients (astrocyte cluster as in FIG. 8C). (FIG. 6C) qPCR analysis of Abca1 expression in Ribotag-isolated astrocytes isolated from GCV-treated WT and Gfap-TK GBM-bearing mice (as in FIGS. 2A-F). (FIG. 6D) Representative immunofluorescence images of sham-injected or GL261-bearing mice stained for ABCA1 (purple), GFAP (reactive astrocytes, yellow), GBM, (GFP⁺-GL261, red), and nuclei (DAPI, white); asterisk indicates the injection coordinates in sham, scale bars, 35 m (n=2 biologically independent experiments, 4 mice per group). (FIG. 6E) Representative immunoblot and quantification analyses comparing ABCA1 protein levels in astrocytes treated with GBM-CM for 24 hours (n=3 biologically independent experiments). (FIG. 6F) Schematic map of the astrocyte-specific shRNA lentiviral vector. (FIGS. 6G-N) Intracranially injection of astrocyte-specific shAbca1 lentivirus attenuates GBM progressions. Non-targeting (Gfap-shNT) or Abca1-targeting (Gfap-shAbca1) astrocyte-specific lentiviruses were injected into the TME of GL261-bearing mice every 5 days (as indicated) starting 9 days after tumor implantation. (FIG. 6G) qRT analysis of FACS-sorted GFP⁺-astrocytes for Abca1 on day 18; expression normalized to Ppia. Data are representative of two independent experiments (n=4 biologically independent samples). (FIGS. 6H-I) Representative flow cytometry plots of filipin III staining in tdTomato+-expression GL261 cells from each group are shown in FIG. 6H and quantification analyses of the percentage of Filipin⁺ GL261 cells and filipin intensity (gMFI) are depicted in FIG. 6I. Data are representative of two independent experiments (n=4 biologically independent samples per group). (FIGS. 6J-K) Representative immunofluorescence images of cleaved caspase-3 (red) and nuclei (DAPI, blue) 10 days after lentivirus injection are shown in FIG. 6J and quantification is depicted in FIG. 6K. T indicates the tumor. Data are representative of two independent experiments (n=4 biologically independent samples per group). (FIGS. 6L-M) Representative images of GL261-derived bioluminescence from each group are shown in FIG. 6L and quantification of tumor size is depicted in FIG. 6M. Data are representative of three independent experiments with n=6 mice/group. (FIG. 6N) Kaplan-Meier curves assessing overall survival of these groups. Data are representative of two independent experiments with n=6 mice/group. (FIG. 6O) Kaplan-Meier curves assessing overall survival of GBM patients based on ABCA1 expression; n represent the number of patients per group. Data are shown as mean±s.e.m. P values were determined by two-sided Student's t-tests (FIGS. 6A, 6C, 6E, 6G, 6H-K), two-way ANOVA (FIGS. 6L-M) or Log rank (Mantel-Cox) test (FIGS. 6N-0). *P<0.05, **P<0.01, ***P<0.001. n.d. not detected.

FIGS. 7A-K illustrate that Tumor-associated astrocyte (TAA) depletion halts glioblastoma progression. (FIGS. 7A-B) Tumor size of GL261-bearing mice as determined by bioluminescence imaging: (FIG. 7A) GL261 cells were intracranially implanted into WT mice. GBM-bearing mice were treated daily with Ganciclovir (GCV, 25 mg/kg) or vehicle (PBS) from day 10 until the experimental end. Data are representative of two independent experiments with n=8 mice/group. (FIG. 7B) GL261 cells were intracranially implanted into WT or Gfap-TK mice. Data are representative of two independent experiments with n=7 mice/group; (FIG. 7C) GfapCRE:iDTR breeding scheme. Mice in which the expression the DT receptor (DTR) from a ubiquitously active promoter is prevented by a loxP-flanked stop cassette (iDTR; induced Diphtheria toxin) were crossed with transgenic mice expressing the Cre recombinase under the control of the GFAP promoter to generate GfapCRE:iDTR mice, in which DTR expression is limited to GFAP⁺ astrocytes, resulting in their depletion following Diphtheria toxin-A (DT-A) administration; (FIGS. 7D and 7G) iDTR or GfapCRE:iDTR littermates, were intracranially implanted with GL261. Ten days later mice were treated daily with DT-A (25 mg/kg). Tumor size was analyzed by bioluminescence imaging. Data are representative of two independent experiments with n=7 mice/group; (FIGS. 7E-F) Kaplan-Meier curves assessing overall survival of mice from FIG. 7A and FIG. 7B, respectively; (FIGS. 7H-K) Astrocyte ablation halts CT-2A glioma pathogenicity. CT-2A glioma cells were intracranially implanted into WT or Gfap-TK littermates and treated with GCV as in (FIGS. 7D and 7G). Tumor growth was analyzed by bioluminescence imaging (FIGS. 7H-I), and mice weight loss and survival were monitored (FIGS. 7J and 7K, respectively). Data in FIGS. 7A, 7B, 7D, 7H, and 7I are shown as mean±s.e.m. P values were determined by two-way ANOVA (FIGS. 7A, 7B, 7D, 7H, and 7I) or Log rank (Mantel-Cox) test (FIGS. 7E, 7F and 7K)). **P<0.01, ***P<0.001.

FIGS. 8A-Q illustrate transcriptomic analysis of tumor-associated astrocytes. (FIG. 8A) PCA of differentially regulated genes of RiboTag-isolated astrocytes (as in FIGS. 2A-F) from PBS-injected mice (Sham, pink) or GL261 GBM-bearing mice (GBM, blue). PC1 was associated with the variance between the sham and GBM data sets, whereas PC2 was associated with the variance between each group; (FIG. 8B) Validation of RNA seq data in different biological samples. qPCR analysis of Cd274, Ccl2, Stat1, Cd74, Cxcl10, Stat3, Csf1, Gpnmb, C3, Cls1, and Mki67 expression in RiboTag-isolated astrocytes from sham-injected or GBM-implanted injected mice; expression normalized to Ppia. Data are representative of 3 independent experiments (n=4 biologically independent experiments). Data are shown as mean±s.e.m. P values were determined by two-sided Student's t-tests. *P<0.05, **P<0.01, ***P<0.001; (FIGS. 8C-D) Analysis of single-cell data of GBM microenvironment performed on data by Darmanis et. al. (discussed herein below). (FIG. 8C) unbiased clustering of the TAMs (1842 cells, orange), TAAs (1052 cells, light blue), oligodendrocyte precursor cells (OPCs, 406 cells, red), oligodendrocytes (81 cells, dark blue), and endothelial cells (50 cells, purple), neurons (21, green) and neoplastic cells (137, gray) defined based on the expression of known markers), presented as color coded TSNE plot. (FIG. 8D) Heat map overly of the scRANseq gene expression intensity within the astrocyte cluster of CHI3L1, CD74, CIS, C3, CD274, GPNMB, ANXA2, MKI67, STAT1, STAT3, AHR, and CSF1. Expression levels are defined by color-coded expression as indicated (from yellow to red; gray coloring indicates that the transcript was not detected). (FIG. 8E) Immunohistochemistry analysis of GFAP (magenta) co-localization with Annexin A2 (ANXA2, yellow), PD-L1 (yellow), Ki67 (yellow), or CD74 (yellow) of sham-injected or GBM-bearing mice. Representative images on the left, box-plot analysis of the antigen-positive astrocytes per cm² (n=9), on the right. Scale bars, 500 m (left), 5 m (right). Co-localization (white) is identified by white arrowheads. Data are shown as median, interquartile interval, minimum, and maximum values. (FIGS. 8F-I) CD8⁺ T-cell depletion in WT or Gfap-TK mice GBM-bearing mice. GL261 cells were intracranially implanted, and the mice were treated daily with GCV from day 9 until the experimental end (as in FIG. 1D), and intraperitoneally injected (black arrows) with CD8 depleting mAbs (αCD8) or isotype control (IC) (0.1 mg/mouse, as in ShiY., et al., Nature 2019, 567: 341-346). Representative data of two independent experiments (n=9 mice/group). (FIGS. 8F-G) Analysis of the CD8+ T-cells frequency in the blood. Representative flow cytometry plots of CD3+/CD8+ staining are shown in FIG. 8F and quantification analyses of CD8+ T-cells frequency are shown in FIG. 8G. (FIGS. 8H-I) Tumor size of GL261-bearing mice as determined by bioluminescence imaging. P values were determined by two-sided Student's t-tests (FIGS. 8B and 8E) or two-way ANOVA (FIGS. 8G and 8H). *P<0.05, **P<0.01, ***P<0.001. (FIGS. 8J-80) Analysis of astrocyte diversity. (FIGS. 8J-K) Sub clustering of astrocytes based on differential expression. (FIG. 8J) Color-coded TSNE plot-, of cluster A (Blue, 599 cells) and cluster B (Pink, 453 cells). (FIG. 8K) Volcano plot of gene expression in astrocytes, color-coded by the cluster enrichment. (FIG. 8L) Top 20 Functional enrichment pathways (FDR<0.05) in the astrocyte clusters, color-coded by the cluster enrichment. (FIGS. 8M-N) Heat map overly of the scRNAseq gene expression intensity of astrocyte immune and cholesterol signatures (FIG. 8M and FIG. 8N, respectively). Expression levels in heatmaps are color-coded (from yellow to red; Grey indicates that the transcript was not detected). Genes associated with each signature are stated at the bottom of the corresponding heat map. Signature score is defined as the sum of all relevant transcripts per cell. (FIG. 8O) Expression levels overlay of significant (FDR<0.001) transcription factors on volcano plot from (FIG. 8K), color-codded by association with cluster A (blue), B (pink), or expressed evenly between the two clusters (pan-astrocyte expression, black). (FIGS. 8P and 8Q) Analysis of FGF2 expression and its correlation to cholesterol synthesis genes (INSG1, SREBF1, SREBF2, ACAT2, HMGCS1, HMGCR, FDPS, FDFT1, and SQLE), which are regulated by astrocyte cell density (Kambach D M, et al., 2017. Oncotarget 8: 14860-14875). (FIG. 8P) Heat map overly of the transcripts expression levels in the astrocytes, color-coded by their cluster association (as in FIG. 8O). (FIG. 8Q) Heat map of the Jaccard correlation index between FGF2 transcript to the cholesterol synthesis genes. Correlation score is noted within each cell. Correlation is color-coded [negative (yellow), natural (blue) and purple (positive)].

FIGS. 9A-G illustrate an analysis of CCR2 pathway in human GBM. (FIGS. 9A-F) Analysis of gene expression (Box plots, FIGS. 9A, 9C and 9E; n=163 GBM patients and 207 normal controls) and survival correlations (Kaplan-Meier Curve, FIGS. 9B, 9D and 9F; n as indicated) for CCR2 (FIGS. 9A-B), CCL2 (FIGS. 9C-D) and CCL7 (FIGS. 9E-F). n represents the number of patients per group. Data are shown as mean±s.e.m. P values were determined by or two-sided Student's (expression data, *P<0.01) or Log rank (Mantel-Cox) tests (survival, P<0.0001). (FIG. 9G) Heat map overly of the scRNAseq gene expression intensity of CCL2, and CCL7 in tumor-associated astrocyte cluster of GBM patients (as in FIG. 8D). Expression levels are defined by color-coded expression (from yellow to red; Grey indicates that the transcript was not detected).

FIGS. 10A-N illustrate a profile of tumor-associated macrophages in GBM. (FIG. 10A) Volcano plot of gene expression in TAMS FACS-sorted from GCV-treated WT (black) and Gfap-TK (pink) GL261-bearing mice FACS-sorted TAM gene expression with P<0.05 (fold change in relative expression as determined by log 2 (GCV-treated/PBS-treated). (FIG. 10B) Box plot analysis of TCGA data of CD274 gene expression in GBM (n=163) and normal (Norm; (n=207) patients. (FIG. 10C) Heat map overly of the scRNAseq gene expression intensity of CD274 in GBM patients (TAM and TAA clusters are as in FIG. 8C). Expression levels are defined by color-coded expression (from yellow to red; Grey indicates that the transcript was not detected). (FIG. 10D) Kaplan-Meier curves assessing overall survival of GBM patient based on CD274 expression; n represent the number of patients per group. Box plot analysis of TCGA data of CSF1 (FIG. 10E) and CSF1R (FIG. 10F) gene expression in GBM (n=163) and normal (Norm; (n=207) patients. (FIG. 10G) Heatmap overlay of CSF1/CSF1R ratio in the GBM TME, based on scRNAseq gene expression. Ratio intensity is present by color; Blue—only CSF1 expressing cells, Yellow—the dual expression of CSF1 and CSF1R, and Red—cells that only express CSF1R. (FIGS. 10H-I) Kaplan-Meier curves assessing overall survival of GBM patient based on CSF1 and CSF1R expression; n represent the number of patients per group. Data are shown as mean±s.e.m. P values were determined by or two-sided Student's (expression data, *P<0.01) or Log rank (Mantel-Cox) tests (survival, P<0.0001). (FIGS. 10J-N) (FIG. 10J) Representative fluorescent images of primary mouse microglial cells (left) and primary astrocytes, stained for GFAP (green), IBA-1 (white), and nuclei (DAPI, magenta). Scale bars, 20 m. (FIG. 10K) Mixed glial cultures were left untreated or subject to mild trypsinization (Lin L, et al., 2017. J. Neuroinflammation. 14:101), isolating the microglial cells [MG (pure). Representative flow cytometry plots of CD11b/GLAST staining from each group are shown on the left and quantification analyses of CD11b+ microglial cell frequencies are on the right (n=4 biologically independent experiments). Data are shown as mean±s.e.m. P values were determined by or two-sided Student's t-test (***, P<0.001). (FIG. 10L) Pure microglial cultures were prepared, treated with astrocytes conditioned medium (ACM) or control medium (Med), and co-cultured with GFP⁺-GL261 cells for 48 hours (as described in FIG. 4G). Representative flow cytometry plots of GFP-gated GL261 cells from each group are shown on the left, and quantification analyses of cell death are on the right (n=2 biologically independent experiments). Data are shown as mean±s.e.m. P=0.678 by two-sided Student's t-test. (FIGS. 10M-N) pure microglial cultures were prepared, treated, and co-incubated with isotype control or CSF1R blocking mAbs (25 g/ml) for 48 hours, as described in FIG. 4K. Microglial cell death and proliferation were then analyzed by LDH assay (FIG. 10M) and CellTrace™ Violet staining (FIG. 10N). Representative flow cytometry plots are shown on the left and quantification analyses of the percentage of proliferating cells in each generation are on the right (n=2). Data are representative of two independent experiments. Data are shown as mean±s.e.m. P values were determined by two-sided Student's t-test; P=0.89 (FIG. 10M), or Two-way ANOVA, followed by Fisher's LSD post-hoc analysis; P>0.99 (FIG. 10N), ns, not significant.

FIGS. 11A-K illustrate that astrocyte-derived cholesterol supports glioma survival. (FIG. 11A) Representative immunofluorescence images of cholesterol accumulation (Filipin III, white) in the GBM tumor (tdTomato⁺ GL261 cells, red), Tumor margins are indicated in yellow (n=3 biologically independent samples). Scale bars, 400 μm (right), 150 μm (left). (FIGS. 11B-E) Real-time changes in the ECAR (FIGS. 11B-C) and OCR (FIGS. 11D-E) of CT-2A glioma cells, cultured in media supplemented with full serum (FCS) or lipoprotein-deprived serum (LPDS) for 18 hours and measured using Seahorse. Oligo, oligomycin; FCCP, carbonyl cyanide4-(trifluoromethoxy) phenylhydrazone; R/A, rotenone plus antimycin A; 2-DG, 2-deoxy-d-glucose. Glycolysis, glycolytic capacity and glycolytic reserve are extracted from ECAR reading, and basal respiration, ATP production, Maximal respiration, and spare respiratory capacity were determined based on OCR. Data are representative of two independent experiments (n=6 technical replicates per experiment). (FIG. 11F) Scheme of cholesterol synthesis inhibition by HMGCR-inhibitor lovastatin. (FIGS. 11G-H) Representative flow cytometry plots and quantification analyses of Annexin V/DAPI staining comparing astrocytes with CT-2A and GL261 cells after a 3 day treatment with the lovastatin (n=3 biologically independent experiments). (FIGS. 11I-J) Representative immunoblot and quantification analyses comparing LDLR protein levels in with CT-2A and GL261 cells cluttered for 2 days in FCS or LPDS supplemented media. (n=3 biologically independent experiments). (FIG. 11K) Box plot analysis of TCGA gene expression for LDLR in normal (n=207) or GBM patients (n=163). n represent the number of patients per group. Data are shown as mean±s.e.m. P values were determined by two-sided Student's t-tests (FIGS. 11B-E and 11I-K) or by one-way ANOVA, followed by Fisher's LSD post-hoc analysis (FIGS. 11G-H). *P<0.05, **P<0.01, ***P<0.001.

FIGS. 12A-F illustrate that astrocyte-derived cholesterol support glioma survival. (FIG. 12A) Scheme of de-novo cholesterol synthesis pathway. (FIGS. 12B-C) qPCR analyses of Hmgcs1, Hmgcr and Dhcr24 expression in astrocytes treated with GBM-CM (FIG. 12B) or co-incubated for 24 hours with GL261 (FIG. 12C); expression normalized to Ppia (n=4 biologically independent experiments). (FIG. 12D) Heat map overly of the scRNAseq gene expression intensity of HMGCS1, HMGCR, and DHC24 in tumor-associated astrocytes from GBM patients (astrocyte cluster as in FIG. 8C). Expression levels are defined by color-coded expression (from yellow to red; Grey indicates that the transcript was not detected). (FIGS. 12E-F) Analysis of LPDS-induced glioma cell death, in the presence or absence of primary astrocytes, by Annexin-V assay. (FIG. 12E) Representative fluorescent images of murine GFP⁺-GL261 and tdTomato⁺-CT-2A glioma cells co-cultured with or without primary mouse astrocytes (n=4 biologically independent experiments). (FIG. 12F) Representative flow cytometry plots and quantification analyses of Annexin V/DAPI staining comparing human U87EGFRvIII glioma cells co-cultured with or human primary astrocytes for 5 days (n=3 biologically independent experiments). Data are shown as mean±s.e.m. P values were determined by two-sided Student's t-tests (FIGS. 12B and 12C). *P<0.05, **P<0.01, ***P<0.001.

FIGS. 13A-L illustrate astrocytic expression of ABCG1, ABCA1, and astrocyte-specific lentiviruses. (FIG. 13A) Box plot analysis of TCGA gene expression for ABCG1 in normal (n=207) or GBM (n=163) patients. n represents the number of patients per group, **P<0.01. (FIG. 13B) Representative immunofluorescence images of Gfap-shNT or Gfap-shAbca1 astrocyte-specific GFP-expressing (yellow) lentiviruses-injected mice stained for GFAP (astrocytes, purple), IBA1 (TAMs, purple), MBP (oligodendrocytes, purple), and NEUN (neurons, purple); arrowheads indicate co-localization only in GFAP⁺ astrocytes, scale bars, 20 m (n=3 biologically independent samples). (FIGS. 13C-J) Primary astrocytes were transduced with RNAi encoding lentiviruses [Non-targeting shRNA (shNT; SEQ ID NO: 1) or Abca1-targeting shRNA (shAbca1; SEQ ID NO: 2); Schematic map of the astrocyte-specific shRNA lentiviral vector in FIG. 6F], or with astrocyte-specific CRISPR-Cas9 lentivirus targeting the luciferase gene (sgLuc2; control; SEQ ID NO: 4), or Abca1 sgAbca1 (#1; SEQ ID NO: 5) and sgAbca1 (#2; SEQ ID NO: 6); Schematic map of the astrocyte-specific CRISPR-Cas9-sgRNA lentiviral vector in FIG. 13C. shRNA and sgRNA sequences are detailed in Table 3 hereinbelow. Transduced astrocytes were then co-cultured with CT-2A glioma cells in LPDS-media for 5 days, and LPDS-induced glioma cell death was determined by Annexin-V assay. (FIGS. 13D-G) Representative immunoblot (FIGS. 13D-E) and quantification analyses (FIGS. 13F-G) comparing ABCA1 protein levels in transduced astrocytes (n=3 biologically independent experiments). (FIGS. 13H-J) Representative flow cytometry plots of CT-2A glioma cells co-cultured for 5 days with transduced astrocytes are shown in FIGS. 13H-J and quantification analyses are shown in FIG. 13J (n=3 biologically independent experiments). Data are shown as mean±s.e.m. P values were determined by a two-sided Student's t-test (FIGS. 13F-G) or by one-way ANOVA (FIGS. 13F-G and J). *P<0.05, **P<0.01, ***P<0.001. (FIGS. 13K-L) Expression of astrocyte-specific GFP-expressing Gfap-shRNA lentiviruses-injected GBM-bearing mice. (FIG. 13K) Representative immunofluorescence images of GFP (green) and GFAP (astrocytes, magenta), scale bars, 400 m. (FIG. 13L) Representative immunofluorescence images of GFP expression (green) and cell makers for TAMs (IBA1, magenta), oligodendrocytes (MBP, magenta), astrocytes (GFAP, magenta), or neurons (NeuN, magenta). Colocalization (white) is identified by white arrowheads. Large scale image on the left, insert (white box) on the right. scale bars, 40 m, scale bar for insert 10 μM. Data are representative of two independent experiments with n=3 mice/group.

FIG. 14 is a schematic illustration of a pharmaceutical approach which uses FDA-approved therapeutic modalities to inhibit cholesterol efflux to brain tumor cells.

FIGS. 15A-D illustrate that cholesterol-lowering drugs inhibit astrocyte-mediated rescue of glioma cells from cholesterol deprivation. Analysis of LPDS-induced glioma cell death, co-cultured with astrocytes, in the presence of absence of cholesterol-lowering drugs, by Annexin-V assay. (FIGS. 15A-C) Representative flow cytometry plots of murine GL261 treated with statins (FIG. 15A) or ABCA1 inhibitors Probucol (FIG. 15B), and Glyburide (FIG. 15C), and this quantification analyses (FIG. 15D). n=2 biologically independent experiments.

FIGS. 16A-E illustrate that Probucol attenuates GBM progression. (FIG. 16A) Illustration of the experimental design for primary and recurrent GBM models. (FIGS. 16B-D) WT mice were intracranially (IC) implanted with GL261 cells. Tumor-bearing mice were treated daily, starting at day 10, with Probucol (30 mg/kg) or vehicle. (FIG. 16B) Tumor growth curve as determined by IVIS, (FIG. 16C) mice weight loss, and (FIG. 16D) Kaplan-Meier curves assess overall survival. p-value by Two-way ANOVA. Data represent Mean±SEM, 8 mice/group. (FIG. 16E) Illustration of the predicted effects of probucol or glyburide in the different models/disease stages.

FIGS. 17A-C illustrate that cholesterol-deprived gliomas are more susceptible to agent-induced apoptosis. Percentage of cell death of GL261 glioma cells cultured in media supplemented with full serum (D5) or lipoprotein-deprived serum (LPDS) and treated with three apoptotic agents [staurosporine (FIG. 17A), doxorubicin (FIG. 17B), or TMZ (FIG. 17C), at indicated concentrations] or vehicle control (mock) for 24 hours; as determined by lactate dehydrogenase (LDH) cytotoxicity assay (n=3 independent experiments). Data are shown as mean±s.e.m. P values were determined by Two-way ANOVA.**** p<0.001.

FIGS. 18A-G illustrate an analysis of BCL-2 family gene expression in GBM and its correlation to cholesterol efflux in the tumor. (FIG. 18A) Before and after plot of BC-2 family gene expression in normal tissue (N, empty circle; “before”) and GBM tumors (T, filled circle; “after”). Underlie of the gene name represents a significant statistical correlation to ABCA1 expression. (FIG. 18B) Correlation plot analysis of CGGA gene expression data between ABCA1 and significantly correlated Bcl-2 family genes. (FIGS. 18C-E) BCL2A1 gene expression in GBM patients. (FIG. 18C) Box plot analysis of TCGA gene expression for BCL2A1 in normal (n=207) or GBM patients (n=163). n represents the number of patients per group; data are shown as mean±s.e.m (FIGS. 18D-E) Correlation plot analysis of ABCA1 and BCL2A1 gene expression in primary GBM patients (FIG. 18D) or recurrent GBM patients (FIG. 18E). (FIGS. 18F-G) Kaplan-Meier curves assess the overall survival of primary (FIG. 18F) or recurrent GBM (FIG. 18G) patients based on BCL2A1 expression; n represents the number of patients per group. P-values were determined by two-sided Student's t-tests (FIG. 18C), Log rank (Mantel-Cox) test (FIG. 18F-G), or person correlation (FIGS. 18A, 18B and 18D-E).

FIGS. 19A-C illustrate that cholesterol-lowering drugs sensitize glioma cells to TMZ treatment. Analysis of TMZ-induced glioma cell death, co-cultured with astrocytes, in the presence or absence of cholesterol-lowering drugs, as determined by Annexin-V assay. (FIGS. 19A-B) Representative flow cytometry plots of murine GL261 treated with statins (FIG. 19A) or ABCA1 inhibitors (FIG. 19B) in combination with TMZ (lower panels) or vehicle (control, upper panels) and their quantification analyses (FIG. 19C). n=2 biologically independent experiments.

FIGS. 20A-C illustrate that Probucol enhances the potency of TMZ therapeutic. WT mice were IC implanted with GL261-Luc2 cells. Tumor-bearing mice were treated with probucol (30 mg/kg), TMZ (2.5 mg/kg), or both (probucol was administrated two days before, during, and two days following TMZ treatment), starting on day 10. (FIG. 20A) Tumor growth curve as determined by IVIS, (FIG. 20B) mice weight loss, and (FIG. 20C) Kaplan-Meier curves assess overall survival. p-value by Two-way ANOVA. Data represent Mean±SEM, 8 mice/group.

FIGS. 21A-D illustrate that PCSK9 overexpression in the cancer microenvironment halts glioma progression. (FIG. 21A) Box plot analysis of TCGA gene expression for PCSK9 in normal (Norm; n=207), GBM patients (n=163), or low-grade glioma (LGG, n=518). n represents the number of patients per group. (FIG. 21B) Astrocytes were transduced with wild type PCSK9-encoding or empty lentivirus, and 24 hours later, the media was collected and transferred to glioma cells. Representative immunoblot analyses comparing LDLR and ACTIN levels in GL261 glioma cells treated with the astrocyte-conditioned media for 24 hours. (n=2 biologically independent experiments). (FIGS. 21C-D) Intracranially injection of PCSK9-encoding lentivirus attenuates GBM progressions. PCSK9-encoding or empty lentiviruses were injected into the TME of GL261-bearing mice 9 and 15 days after tumor implantation. (FIG. 21C) Analysis of tumor size, based on tumor bioluminescence. (FIG. 21D) Kaplan-Meier curves assessing overall survival of these groups. Data are representative of two independent experiments with n=6 mice/group. Data are shown as mean±s.e.m. P values were determined by two-sided Student's t-tests (FIG. 21A), two-way ANOVA (FIG. 21C), or Log rank (Mantel-Cox) test (FIG. 21D). *P<0.05, **P<0.01, ***P<0.001.

### DESCRIPTION OF SPECIFIC EMBODIMENTS OF THE INVENTION

The present invention, in some embodiments thereof, relates to targeting the lipid synthesis in astrocytes and, more particularly, but not exclusively, to the use of same for the treatment of brain tumors.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Malignant brain tumors are the cause of a disproportionate level of morbidity and mortality among cancer patients, an unfortunate statistic that has remained constant for decades. Despite considerable advances in the molecular characterization of these tumors, targeting the cancer cells has yet to produce significant advances in treatment.

While reducing the present invention to practice, the present inventor has uncovered that astrocytes control glioblastoma pathogenicity by reprogramming the immunological properties of the tumor microenvironment and supporting the non-oncogenic metabolic dependency of glioblastoma on cholesterol.

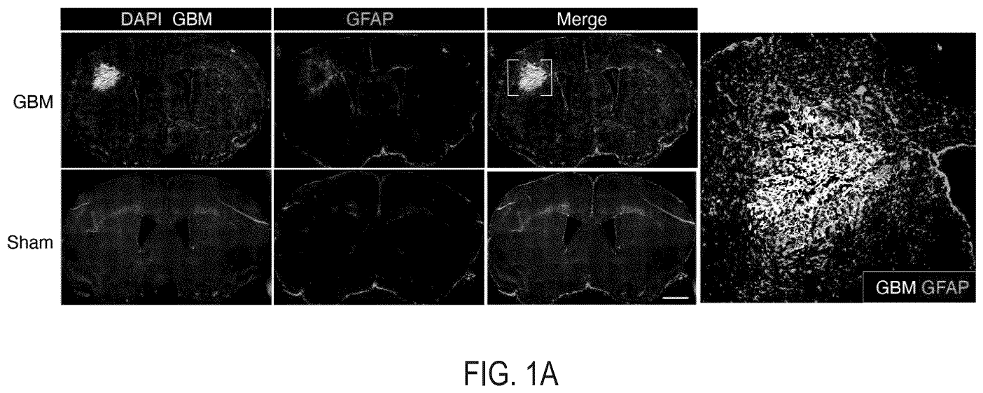

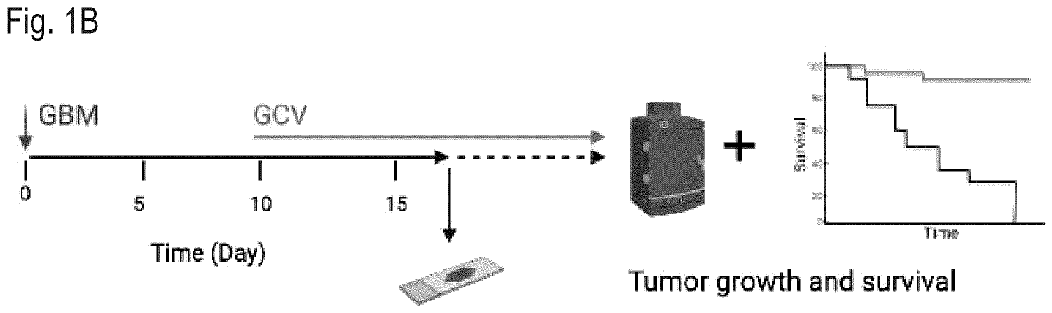

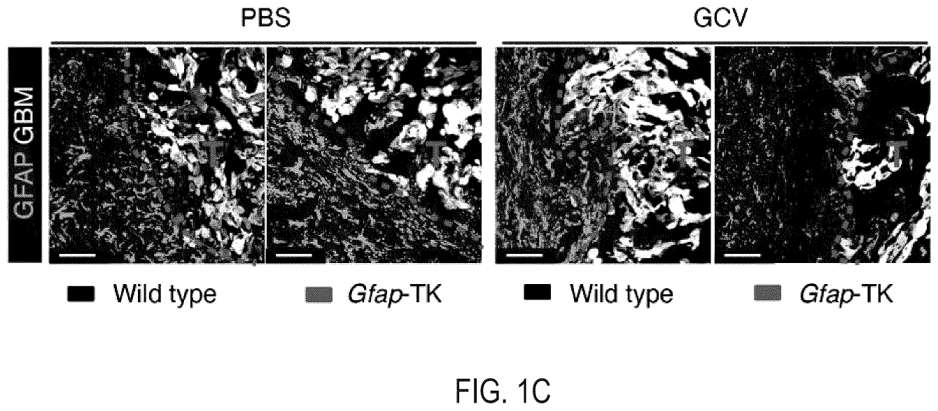

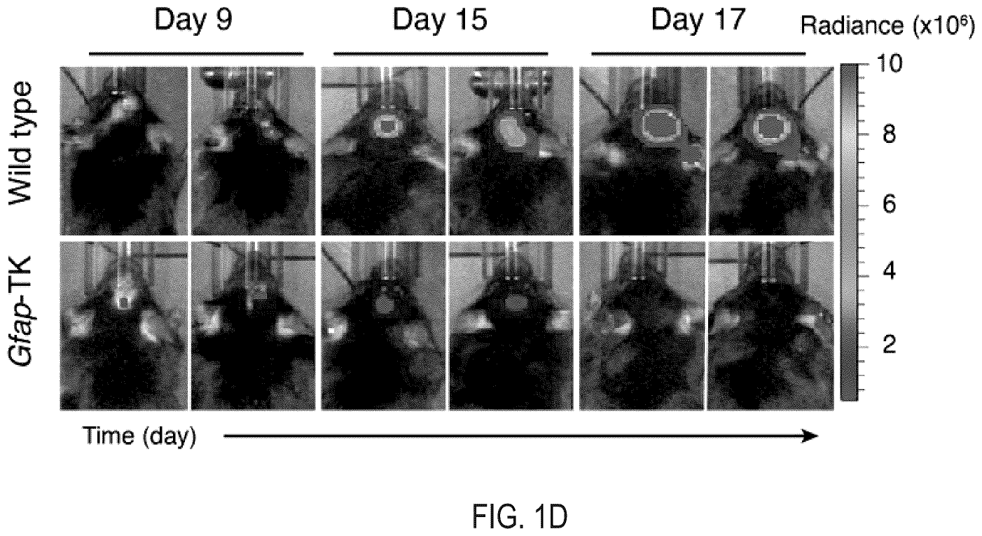

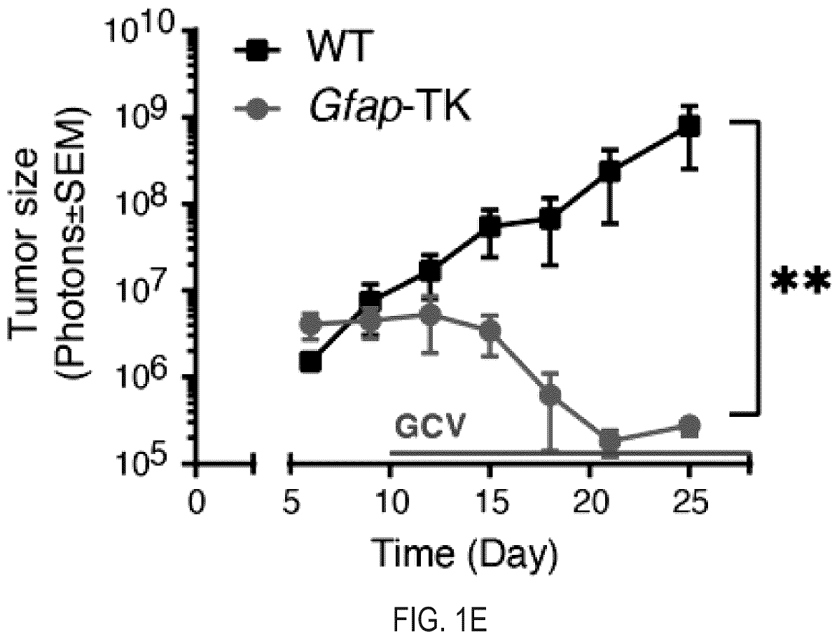

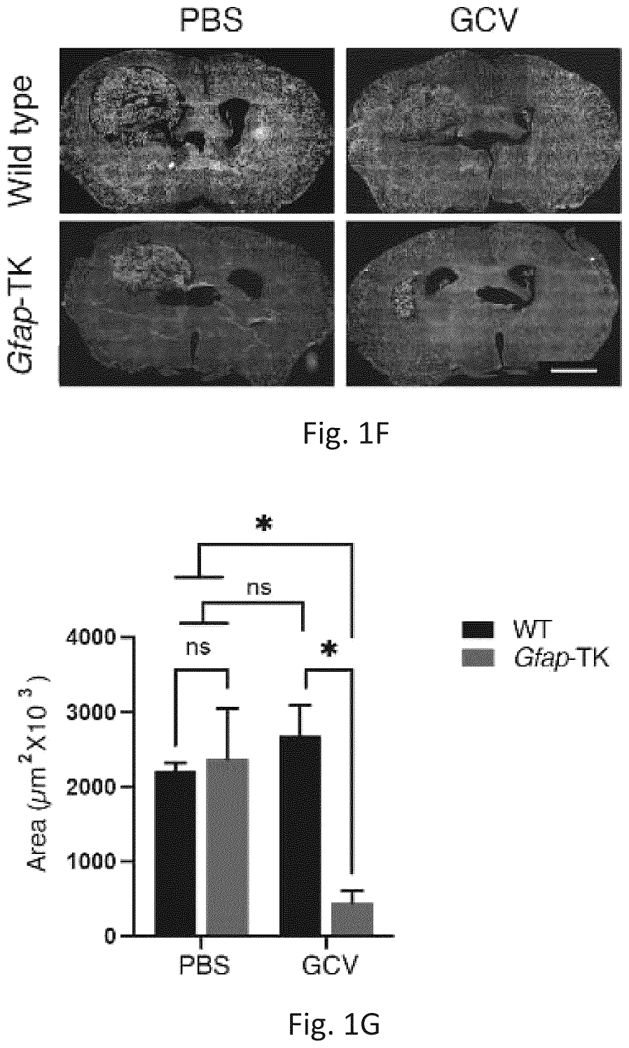

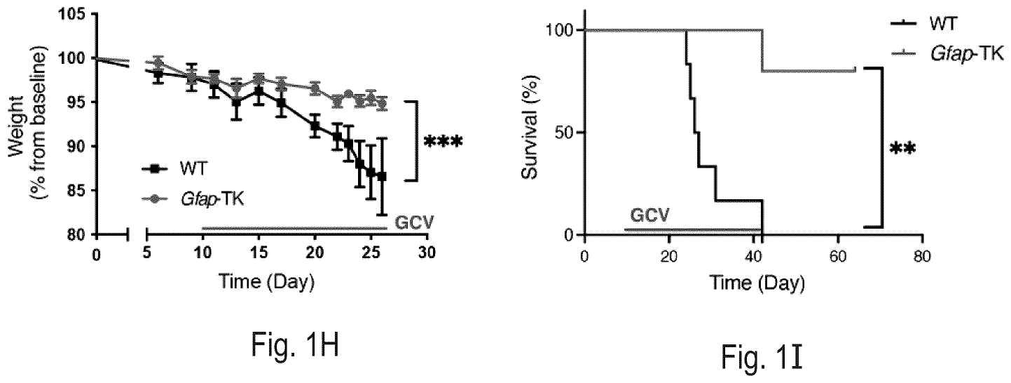

Example 1 of the Examples section which follows shows that reactive tumor associated astrocytes (TAAs) play a pivotal role in supporting glioma progression and tumor pathogenicity, and that depletion of TAAs halts glioma growth, significantly attenuates the weight loss in glioma-bearing subjects, and improves their survival (FIGS. 1A-I). Thus, the present inventor has uncovered that targeting astrocytes in the brain tumor microenvironment can regress tumor growth and prolong patient's survival.

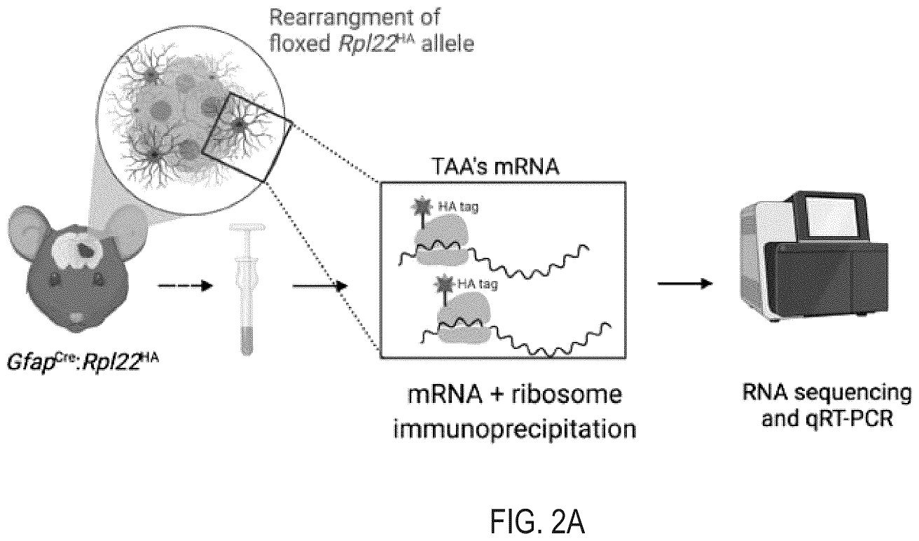

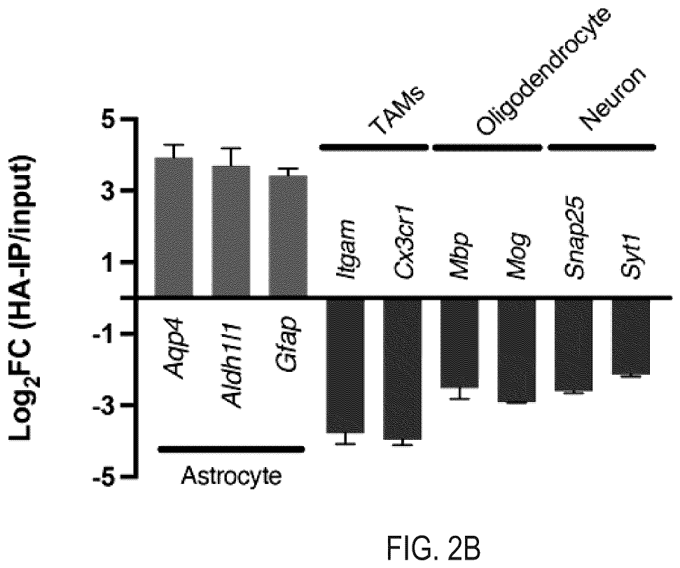

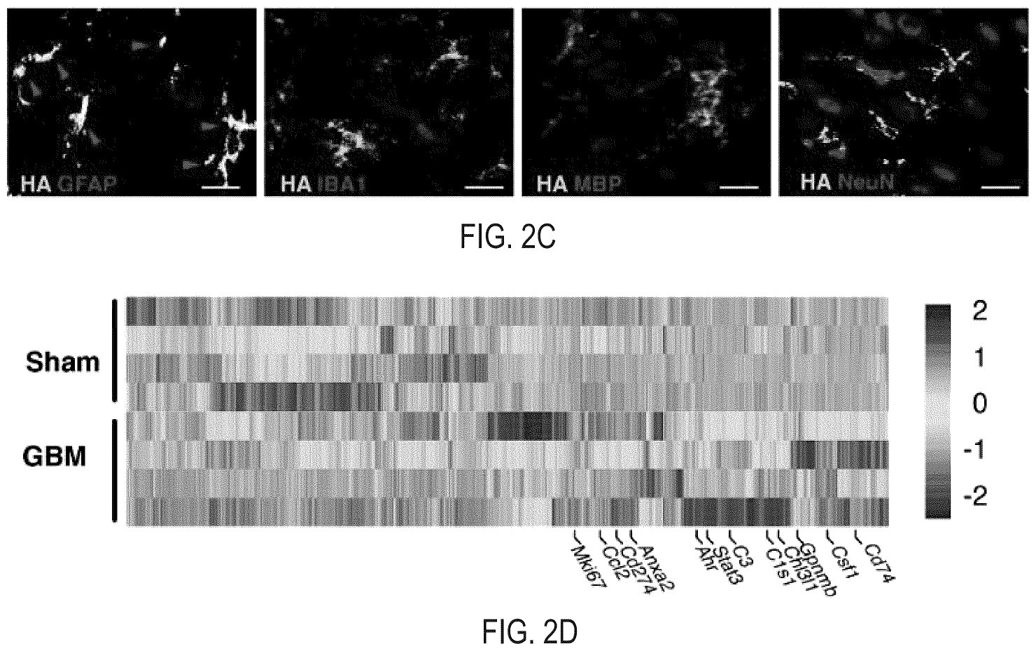

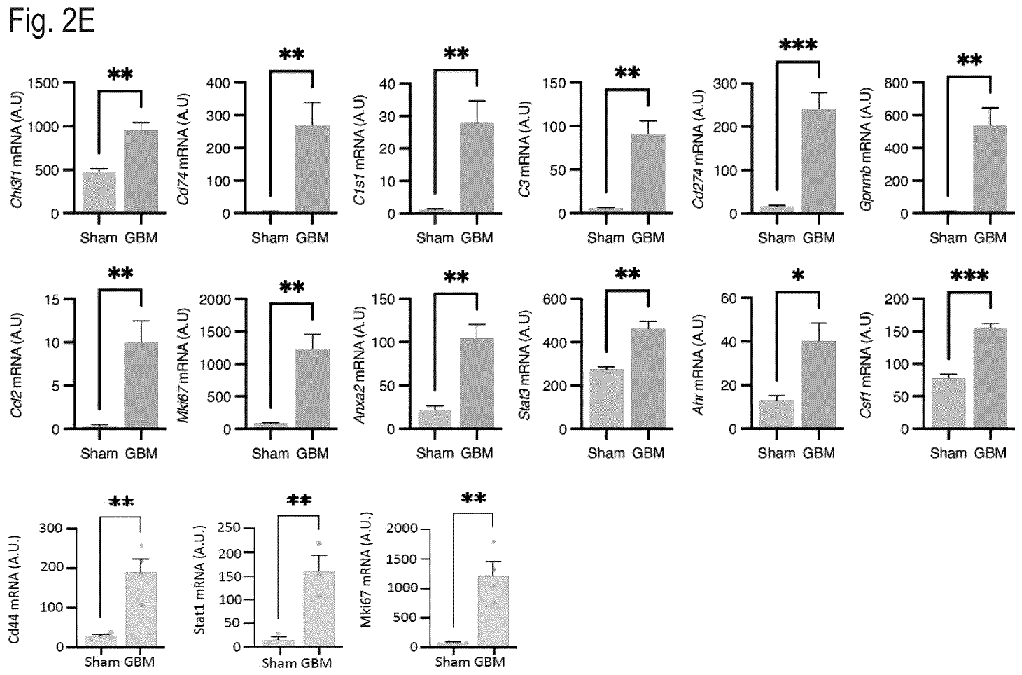

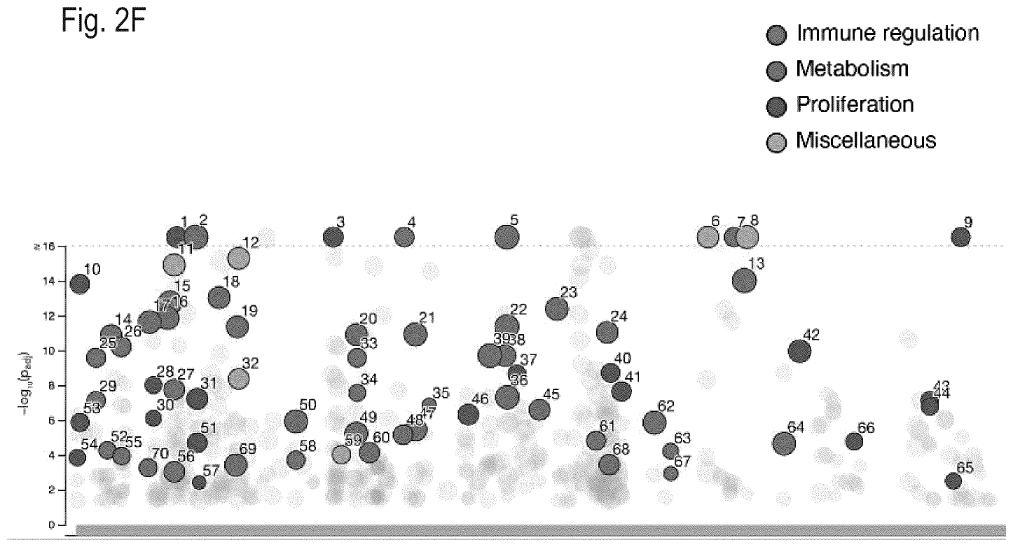

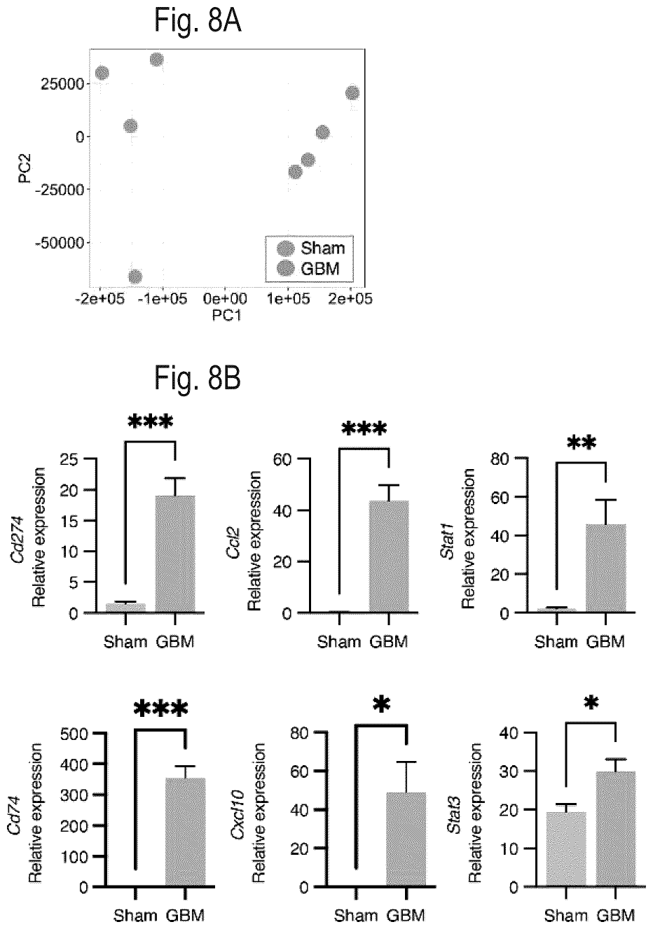

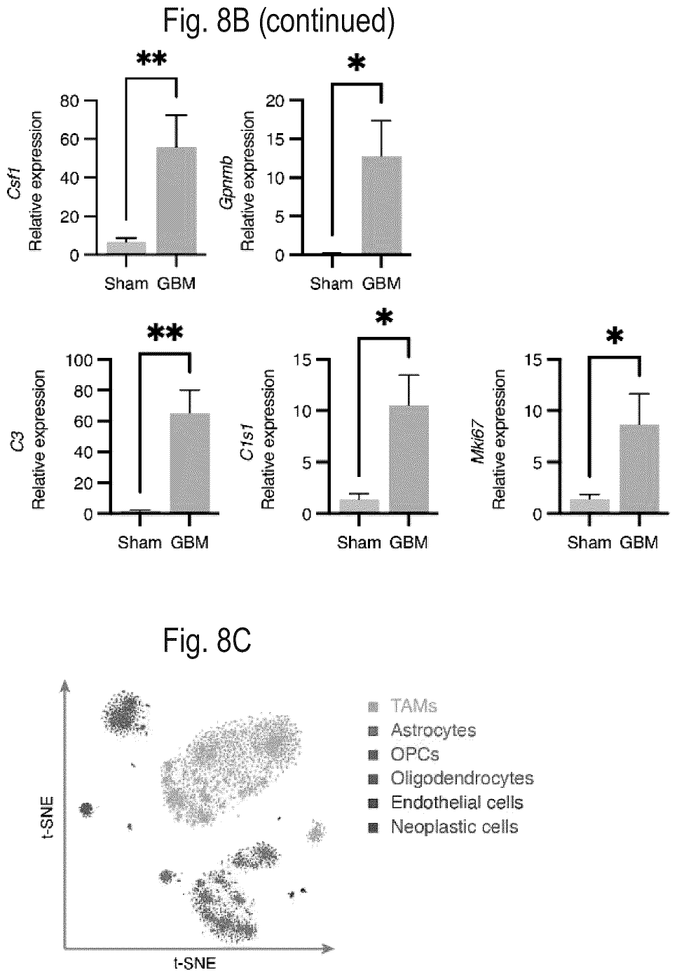

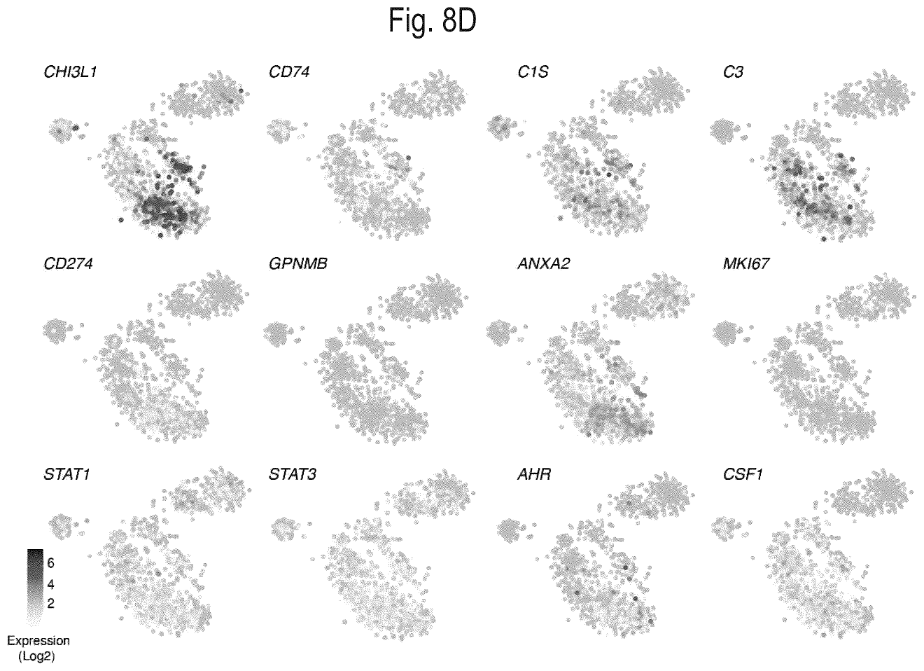

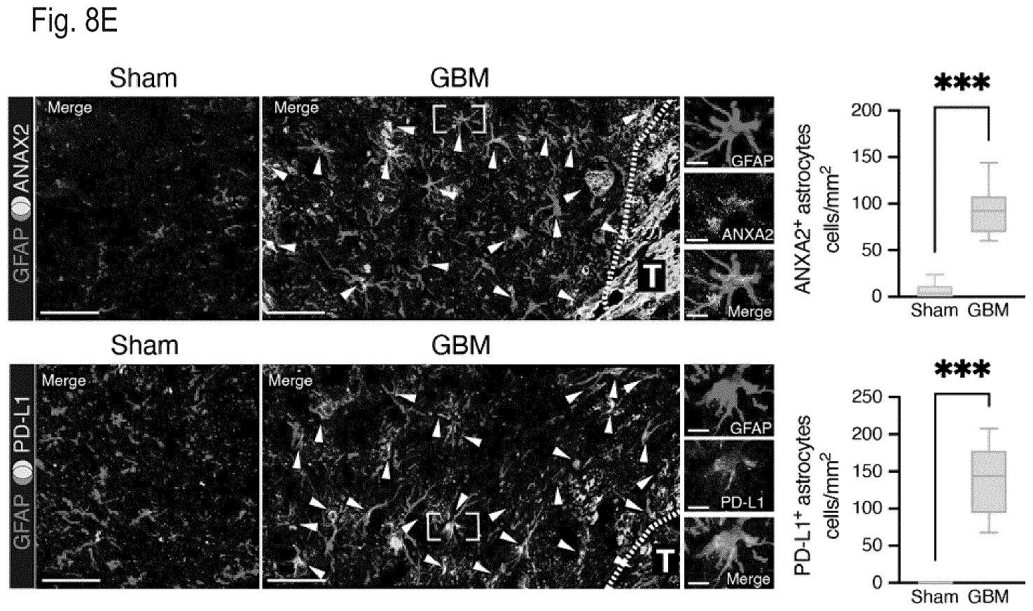

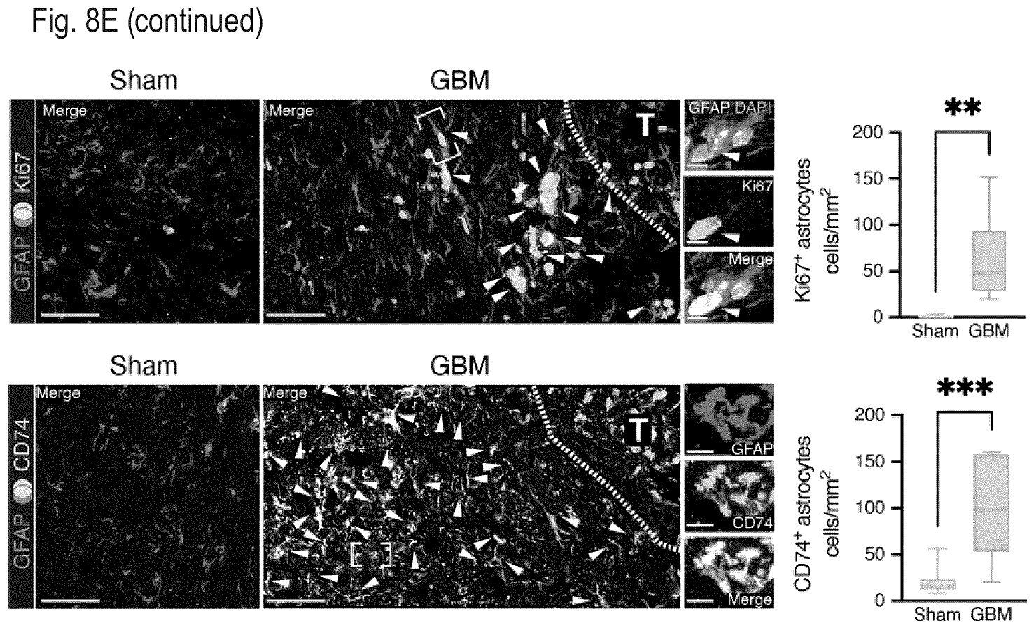

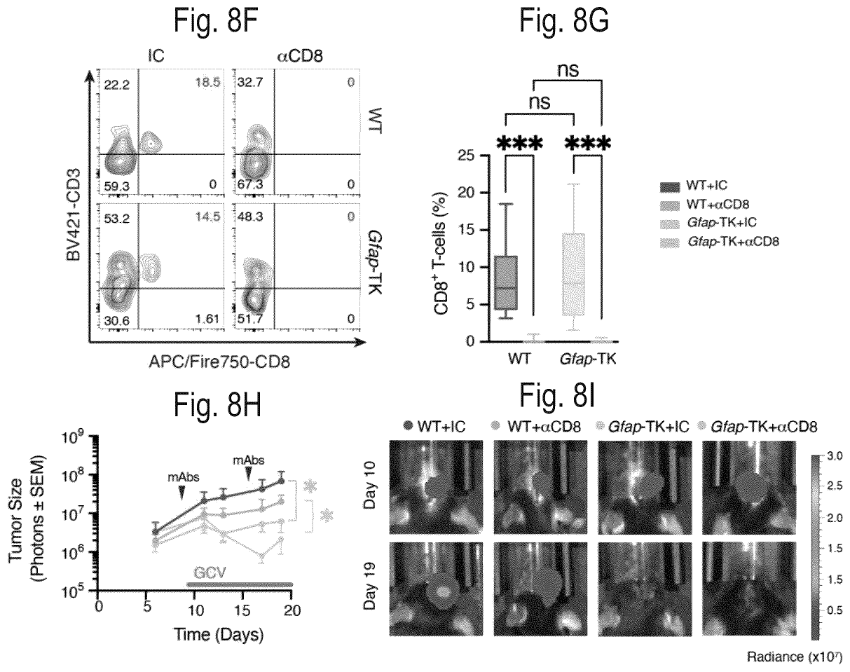

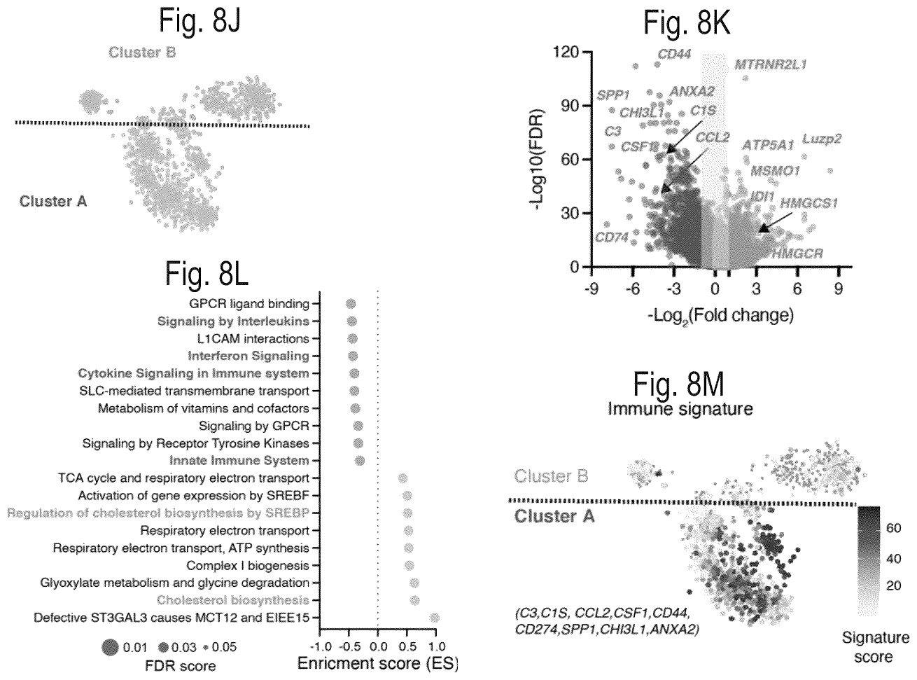

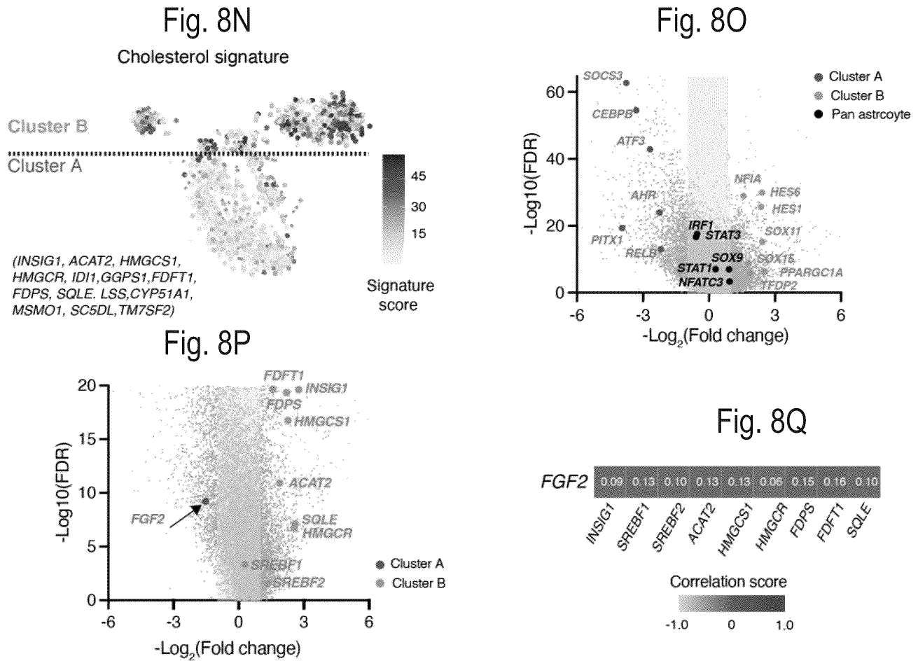

Specifically, the present inventor has analyzed the tumor-associated astrocytes (TAAs) translatome, and uncovered that TAAs in GBM exhibit increased expression of immune-associated genes (Chi3l1, Cd74), complement components (C1s1, C3), immunosuppression (e.g., Cd274, Gpnmb), chemokines (Ccl2), and proliferation (Mki67, Anxa2), as well as genes encoding for transcription factors associated with glial support of brain tumors (Stat3, Ahr) or genes associated with astrocyte crosstalk with microglial cells (Csf1) (FIG. 2E and FIG. 8B). Thus, the present inventor uncovered that astrocytes support glioma pathogenicity by directly promoting immunosuppression, regulating the neighboring immune cells, and contributing to the metabolic landscape of the tumor microenvironment.

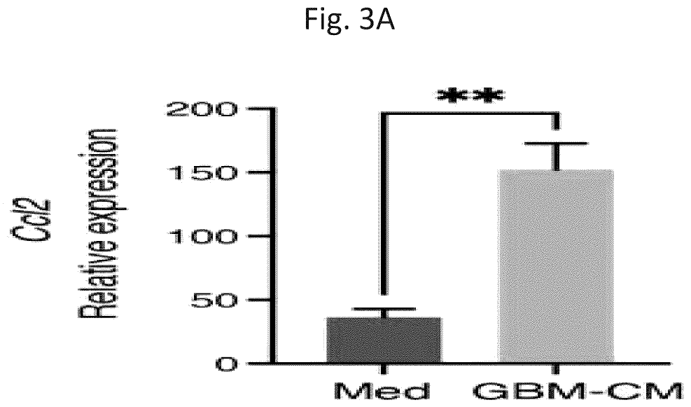

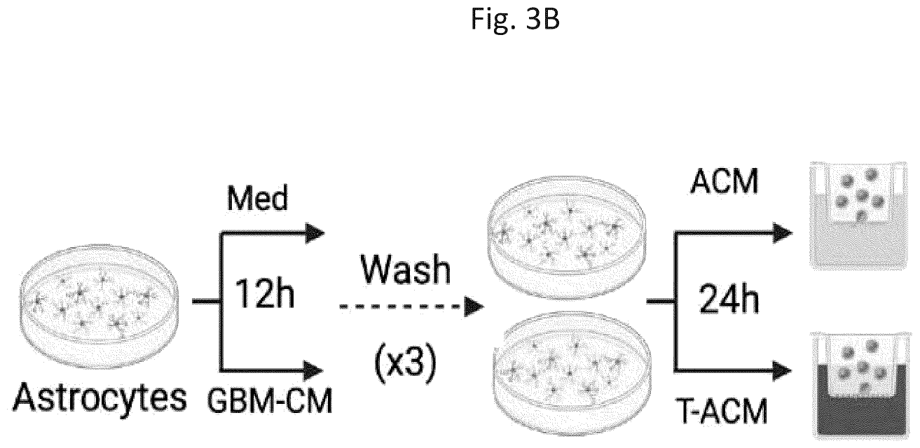

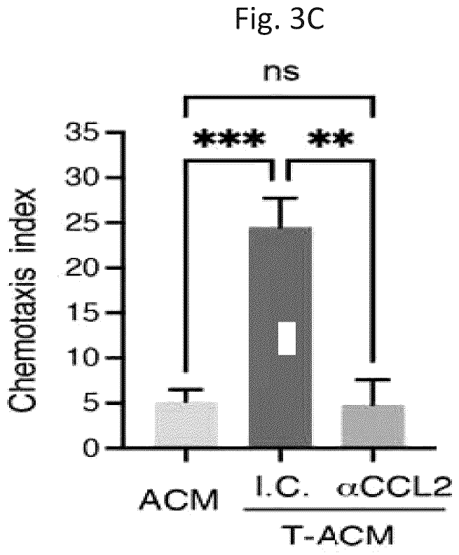

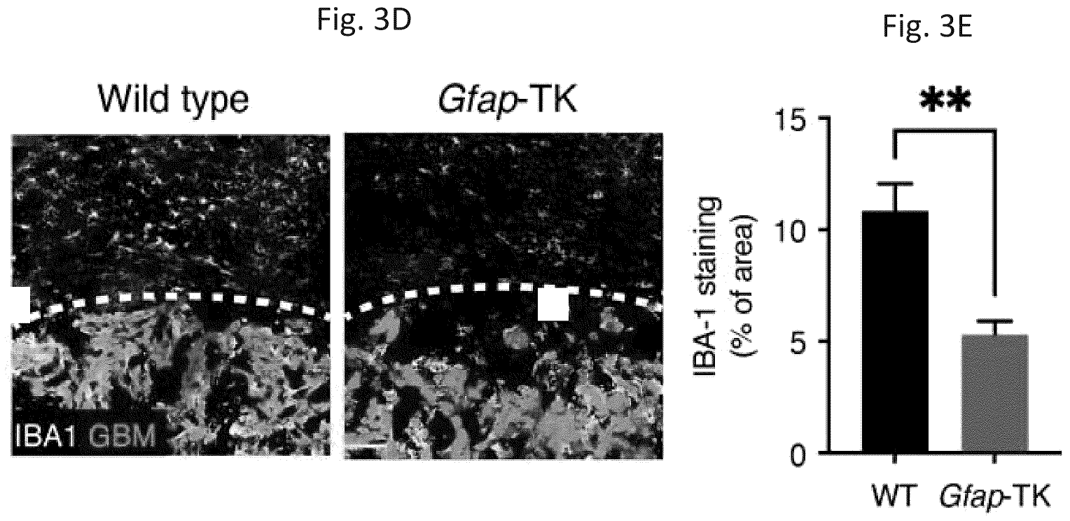

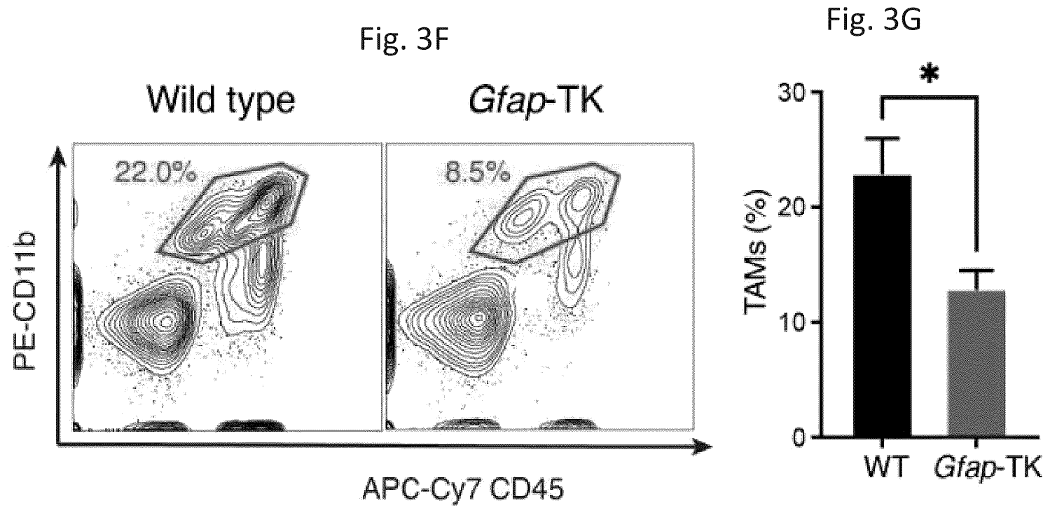

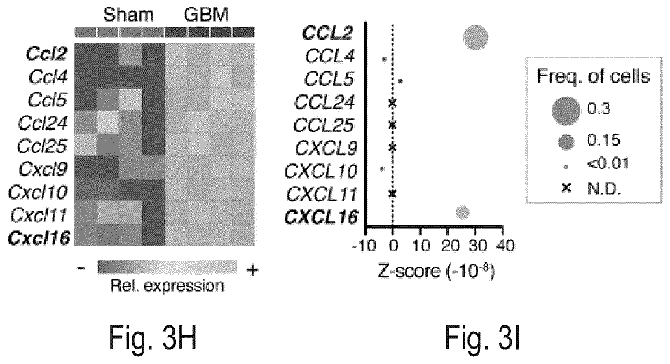

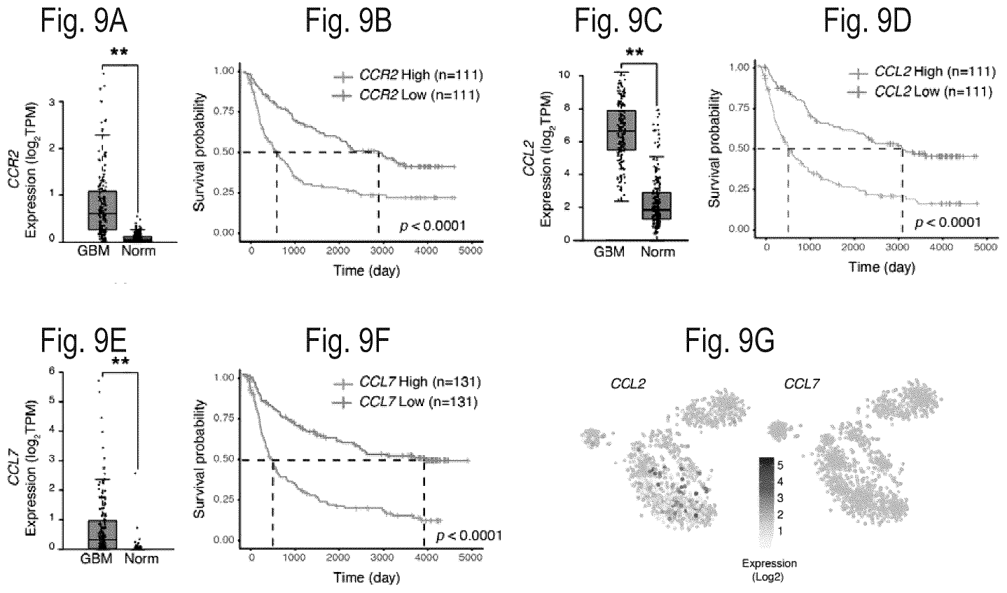

The present inventor has further uncovered presence of high levels of CCL2 in astrocytes from GBM patients (FIG. 9G), in TAAs isolated from GL261-bearing mice (FIG. 2E) and in primary astrocytes treated with tumor-conditioned media generated using murine GL261 glioma (GBM-CM) (FIG. 3A). In addition, while astrocyte condition media (ACM) was efficient in inducing monocyte migration (FIG. 3B) anti-CCL2 neutralizing antibodies inhibited this increase in monocyte migration, implicating CCL2 as the main chemoattractant in GBM-induced astrocyte recruitment of the monocytes (FIG. 3C). In addition, the present inventor has uncovered that depletion of reactive astrocytes with GCV significantly reduced the accumulation of IBA-1⁺ Tumor-associated macrophages (TAMs) in the tumor microenvironment (TME) (FIGS. 3D-E, 3F-G), suggesting that reactive astrocytes directly control the recruitment of TAMs to the GBM TME and that this process is predominantly mediated by CCL2. Taken together, these results reveal that astrocytes initiate transcriptional programs that shape the immune and metabolic compartments in the glioma microenvironment (Examples 1 and 2, hereinbelow).

Specifically, TAA's expression of CCL2 and CSF1 governs the recruitment of Tumor-associated macrophages (TAMs) and promotes a pro-tumorigenic macrophage phenotype (Examples 3 and 4, hereinbelow). Concomitantly, the present inventor has demonstrated that astrocyte-derived cholesterol is key to glioma cell survival, and that targeting astrocytic cholesterol efflux, via ABCA1 (ATP binding cassette subfamily A member 1), halts tumor progression (Examples 5 and 6, hereinbelow). Taken together, these findings elucidate the role of astrocytes in glioblastoma pathogenesis, define the molecular circuits by which the astrocytes shape the immunometabolic landscape of the TME and control tumor progression, thereby identifying astrocytes as a target for therapeutic intervention.

The present inventor has uncovered that downregulation of endogenous synthesis of cholesterol in astrocytes (e.g., reactive astrocytes in the TME) can be used to reduce tumor growth and treat glioblastoma. This is particularly beneficious since lipoprotein-bound cholesterol does not cross the blood brain barrier (BBB)), and since glioma cells cannot de-novo synthesize cholesterol

According to an aspect to some embodiments of the invention, there is provided a method of treating a brain tumor in a subject in need thereof, the method comprising administering to the subject a therapeutically effective amount of an agent capable of specifically downregulating activity or expression of a component of the lipid synthesis and/or transportation pathways in an astrocyte in the tumor microenvironment, wherein the agent is specific to the astrocyte in the tumor microenvironment and not to a cancerous cell of the brain tumor, thereby treating the brain tumor in the subject.

According to an aspect to some embodiments of the invention, there is provided a therapeutically effective amount of an agent capable of specifically downregulating the activity or expression of a component of the lipid synthesis and/or transportation pathways in an astrocyte in the tumor microenvironment, wherein the agent is specific to the astrocyte in the tumor microenvironment and not to a cancerous cell of the brain tumor, for use in treating a brain tumor in a subject in need thereof.

The term “brain tumor” as used herein refers to an abnormal growth of cells intracranially, i.e., within the brain that can be benign or malignant, including abnormal growth of cells that originate and comprise the brain e.g., neurons, glial cells, astrocytes, oligodendrocytes, ependymal cells, lymphatic tissue, blood vessels, cranial nerves, pituitary gland and/or pineal gland, as well as metastases from cancers that originate from other organs, e.g., breast or prostate cancer. Brain tumors that arise from brain tissues include gliomas and non-gliomas. Specific examples of gliomas include astrocytomas, oligodendrogliomas and ependymomas. Non-glioma brain tumors include benign tumors, such as pituitary adenomas and malignant tumors, such as medullblastomas, primary CNS lymphomas, and CNS germ cell tumors.

According to some embodiments, the brain tumor is a malignant brain tumor.

According to some embodiments of the invention, the brain tumor is selected from the group consisting of Acoustic neuroma, Astrocytoma, Choroid plexus carcinoma, Craniopharyngioma, Embryonal tumor, Ependymoma, Glioblastoma, Glioma, Medulloblastoma, Meningioma, Oligodendroglioma, Pediatric brain tumor, Pineoblastoma, Pituitary tumor and Brain metastasis.

According to some embodiments of the invention, the brain tumor is Glioblastoma.

According to some embodiments, the brain tumor is a glioblastoma multiforme (GBM), i.e. an astrocytic tumor that includes giant cell glioblastoma and gliosarcoma.

According to some embodiments of the invention, the subject is a human subject.

The term “tumor microenvironment” or “TME” refers to the area surrounding the brain tumor. Tumor microenvironment typically comprises blood vessels, immune cells, neuronal cells, glial cells of the CNS, signaling molecules, and the extracellular matrix. The glial cells of the CNS, which are part of the TME, may include microglial cells, astrocytes, and oligodendrocytes.

As used herein the phrase “an astrocyte in the tumor microenvironment” refers to a non-cancerous cell.

According to some embodiments of the invention, the astrocyte in the tumor microenvironment comprises a reactive astrocyte.

The term “reactive astrocyte” refers to a non-cancerous astrocyte that undergoes morphological, molecular, and functional changes in response to a pathological situation in a surrounding tissue. Reactive astrocytes can typically be found in the microenvironment of brain tumors.

Reactive astrocytes can be recognized by elevated levels of glial fibrillary acidic protein (GFAP).

GFAP, is one of the major intermediate filament proteins of mature astrocytes. It is used as a marker to distinguish astrocytes from other glial cells during development. Similarly to other intracellular proteins, GFAP is typically expressed on the astrocyte cell surface in a MHC-I restrictive manner. This MHC-I-protein expression complex is referred to as “reactive astrocyte MHC-I complex”.

According to some embodiments of the invention the agent specifically downregulates the activity or expression of a component of the lipid synthesis and/or transportation pathways in astrocytes in the TME rather than in cancerous cells.

According to some embodiments of the invention the agent of some embodiments of the invention completely inhibits the activity or expression of a component of the lipid synthesis and/or transportation pathways in astrocytes in the TME but not in the cancer cells of the brain tumor.

According to some embodiments of the invention, the agent which is capable of specifically downregulating activity or expression of a component of the lipid synthesis and/or transportation pathways in an astrocyte in the tumor microenvironment binds directly or indirectly to an astrocyte in the TME (e.g., a reactive astrocyte in the TME) and not to a cancerous cell of the brain tumor.

As mentioned, the agent is capable of downregulating activity or expression of a component of the lipid synthesis and/or transportation pathways in an astrocyte.

The phrase “component of the lipid synthesis and/or transportation pathways” refers to any molecule that directly or indirectly affects the rate of lipid (e.g. cholesterol) biosynthesis or transport (e.g. across the cell membrane). For example, any molecule that affects the rate of lipid (e.g. cholesterol) biosynthesis (e.g. via the mevalonate pathway, e.g. also referred to as the de-novo cholesterol synthesis, or Bloch pathway) by catalyzing lipid (e.g. cholesterol) biosynthesis, affecting the localization or accumulation levels of precursors of such lipids, affecting the rate of activity of enzymes that catalyze such biosynthesis, the accumulation levels of such enzymes, the availability or localization of these enzymes. In addition, any molecule that affects the rate of lipid (e.g. cholesterol) transport, such as cholesterol receptors (e.g., low-density lipoprotein receptor, LDLR), molecules mediating the efflux of cholesterol and phospholipids across the cell membrane (e.g. ABC transporters, e.g. ABCA1), molecules facilitating lipid (e.g. cholesterol) transfer across the cytosol, such as sterol transfer proteins (STPs) and Apolipoproteins (e.g. Apo B, Apo CIII).

The phrase “an agent capable of downregulating activity or expression of a component of the lipid synthesis and/or transportation pathways” refers to a molecule that downregulates an activity or expression of a component of the lipid synthesis and/or transportation pathways per se (i.e., direct inhibition) or a down-stream signaling effector (i.e., indirect inhibition) thereof.

As used herein “activity of a component of the lipid synthesis and/or transportation pathways” refers to an enzymatic activity, localization activity, transport activity, efflux activity of any molecule that affects the rate of lipid (e.g. cholesterol) biosynthesis or transport.

As used herein “expression of a component of the lipid synthesis and/or transportation pathways” refers to expression of mRNA or protein products of a molecule that affects the rate of lipid (e.g. cholesterol) biosynthesis or transport, e.g. expression of enzymes, of precursors of enzymes, of lipid binding molecules, or of transport molecules.

According to some embodiments of the invention, the lipid is selected from the group consisting of a cholesterol, a cholesteryl ester (CE), a triglyceride and a sphingolipid.

According to some embodiments of the invention, the lipid is cholesterol.

According to some embodiments of the invention, the agent is an efflux inhibitor.

The term “efflux” when relating to a lipid (e.g., cholesterol) refers to the transfer of a lipid from one cell or an immediate environment thereof to another cell.

For example, the transfer of a lipid (e.g., cholesterol) from an astrocyte to a cancerous cell; or the transfer of a lipid from the tumor microenvironment to a cancerous cell of the brain tumor.

According to some embodiments of the invention, the agent is an efflux inhibitor which inhibits release of a lipid from the astrocyte in the tumor microenvironment.

According to some embodiments of the invention, the efflux inhibitor inhibits the release of a lipid from the astrocyte in the TME to the cancerous cells of the brain tumor, and thus blocks the supply of lipid(s) to the cancerous cells of the brain tumor.

According to some embodiments of the invention, the efflux inhibitor is an inhibitor of the ATP-binding cassette transporter A1 (ABCA1).

According to some embodiments of the invention, the component of the lipid synthesis or transportation pathway is an ATP-binding cassette transporter A1 (ABCA1).

ABCA1 is a membrane-associated protein which is a member of the superfamily of ATP-binding cassette (ABC) transporters, which transport various molecules across extra- and intracellular membranes. With cholesterol as its substrate, ABCA1 protein functions as a cholesterol efflux pump in the cellular lipid removal pathway.

According to some embodiments of the invention, the component of the lipid synthesis or transportation pathway is a molecule associated with the de-novo cholesterol synthesis pathway.

According to some embodiments of the invention, the component of the lipid synthesis or transportation pathway is a molecule associated with cholesterol catabolism to oxysterols.

According to some embodiments of the invention, the component of the lipid synthesis or transportation pathway is a molecule associated with liver-X-receptors (LXRs).

According to some embodiments of the invention, the component of the lipid synthesis or transportation pathway is a molecule associated with oxysterols catabolism.

According to some embodiments of the invention, the component of the lipid synthesis or transportation pathway is a molecule associated with cholesterol efflux.

According to some embodiments of the invention, the component of the lipid synthesis or transportation pathway is a molecule associated with cholesterol uptake.

According to some embodiments of the invention, the component of the lipid synthesis or transportation pathway is a molecule associated with the induction of Mylip.

According to some embodiments of the invention, the agent of some embodiments of the invention is a small molecule which is capable of downregulating activity or expression of a component of the lipid synthesis and/or transportation pathways in an astrocyte in the tumor microenvironment.

There are at least two main families of FDA-approved drugs that may control cholesterol efflux to the glioma cells: 1) Statins, which inhibit cholesterol synthesis, and 2) Probucol and glyburide that inhibit the activity of the cholesterol transporter ABCA1.

Without being bound to any theory, the present inventor envisages that drug-mediated inhibition of cholesterol synthesis, and/or inhibition of ABCA1 activity in an astrocyte of the TME would limit cholesterol efflux to the tumor cells, depriving the cancerous cells of vital energy sources and building blocks, and thus lowering their apoptotic threshold, and attenuating tumor survival.

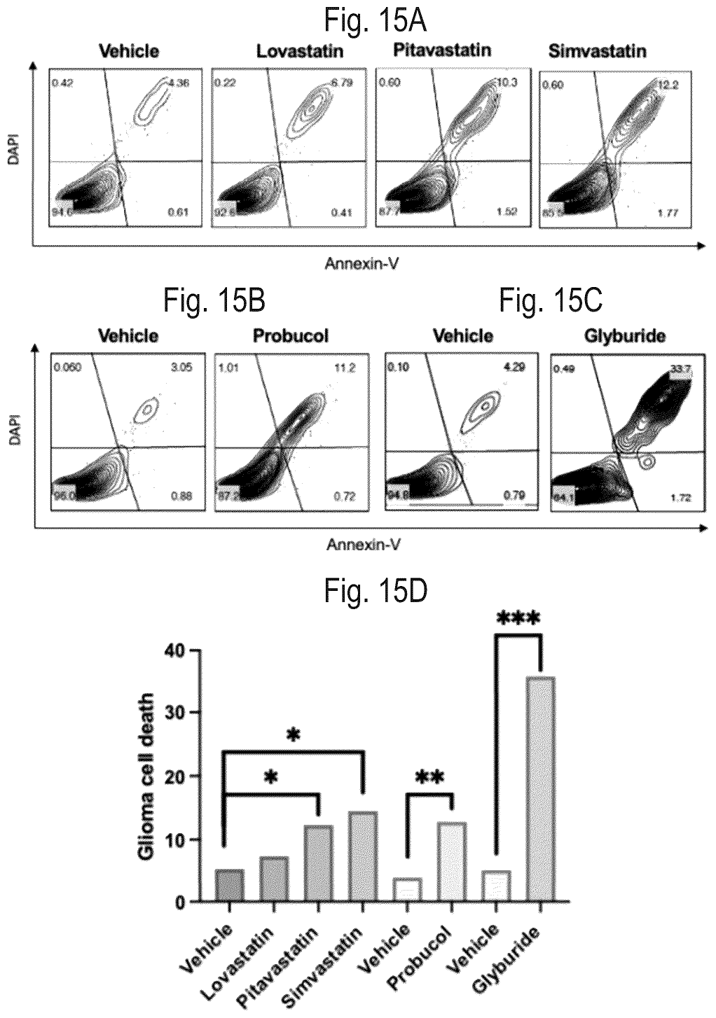

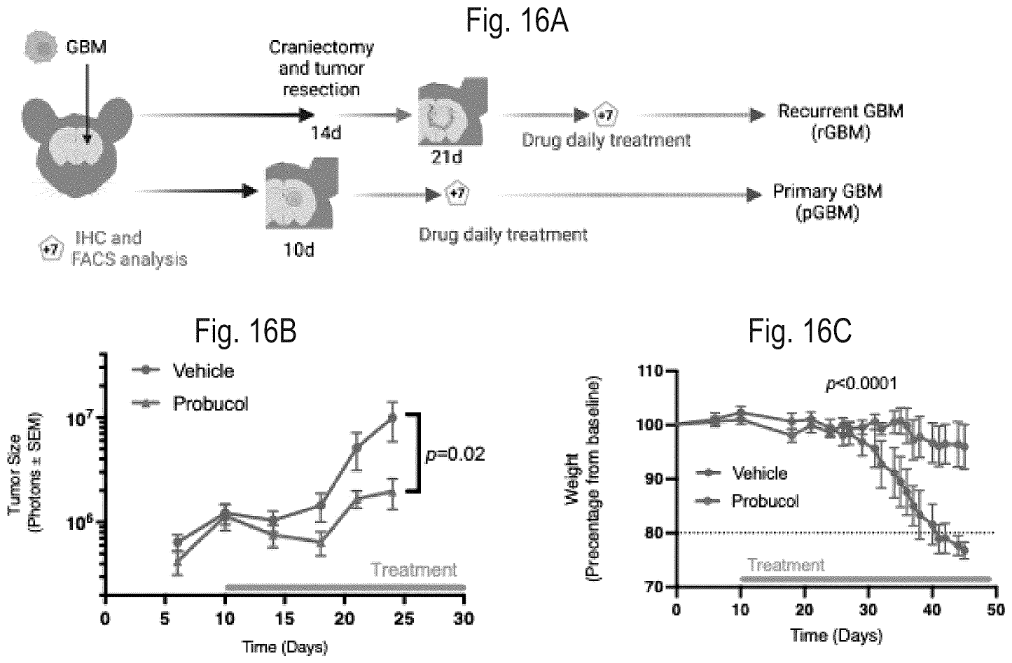

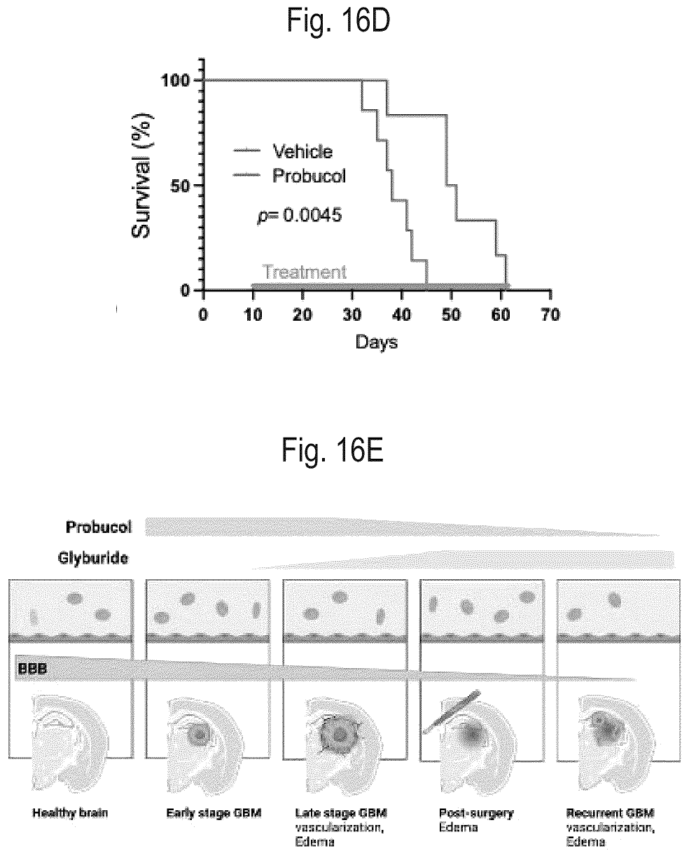

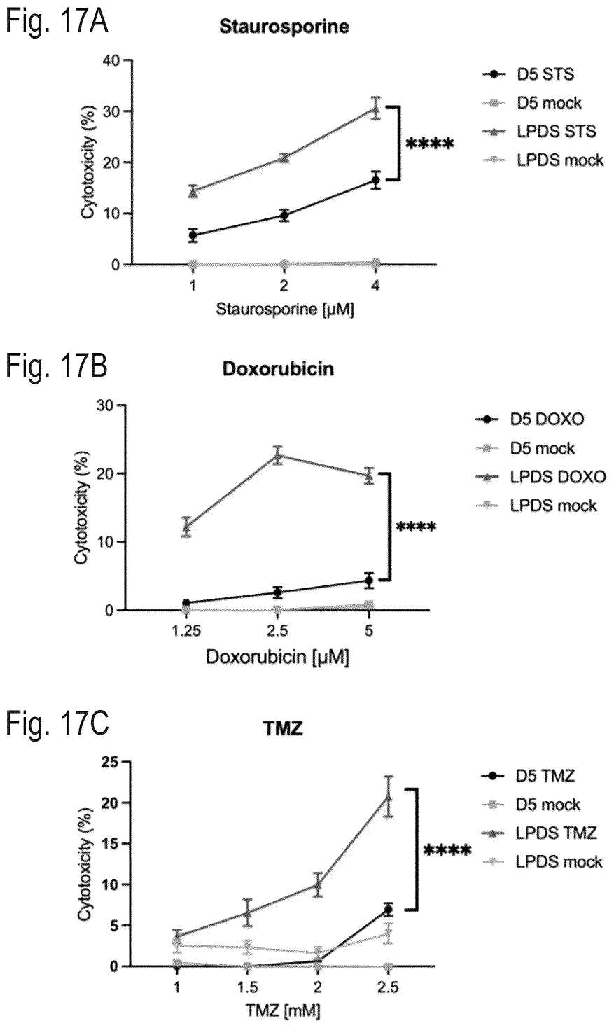

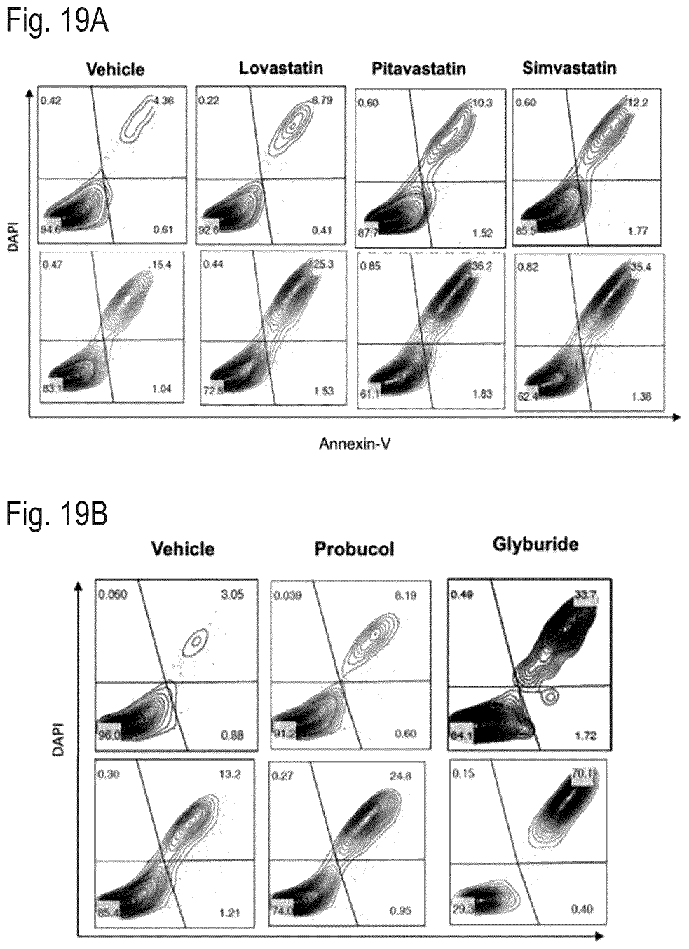

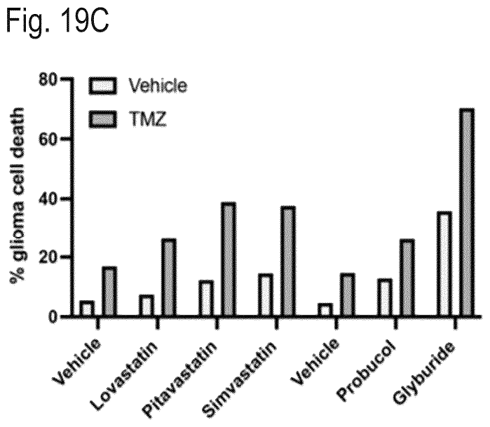

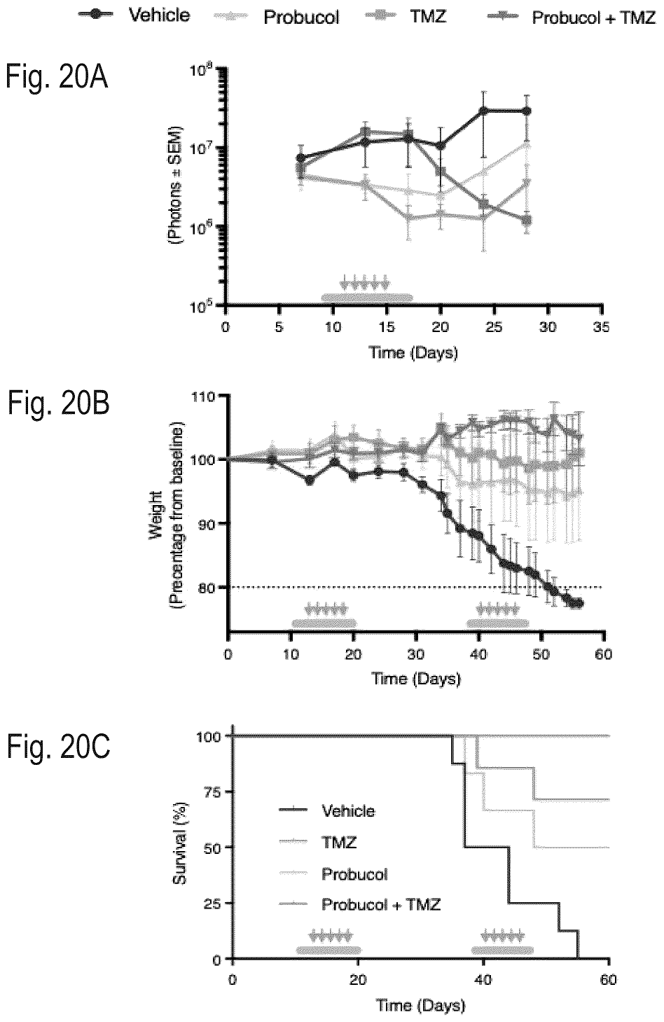

Indeed, Examples 7-10 of the Examples section which follows show that statins and ABCA1 inhibitors were capable of inducing glioma cell death in-vitro (FIGS. 15A-D); that ABCA1 inhibitors (e.g., Probucol and Glyburide) were capable of decreasing tumor size, preventing weight loss and prolonging survival of subjects having GBM in-vivo (FIGS. 16A-E and Examples 8 and 9 of the Examples section which follows); that cholesterol-deprived gliomas are more susceptible to agent-induced apoptosis (FIGS. 17A-C); that cholesterol-lowering drugs sensitize glioma cells to TMZ treatment in-vitro (FIGS. 19A-C); and that ABCA1 inhibitors (e.g., Probucol) enhances the potency of TMZ therapeutic in GBM in-vivo (FIGS. 20A-C).

According to some embodiments of the invention, the small molecule is selected from the group consisting of a Probucol, Glyburide, a LDLR antisense/decoy molecule, a Menin inhibitor, a Statin, AY-9944, D-003, Avasimibe, Nystatin, Ezetimibe, Fenofibrate, 2-Hydroxypropyl-β-cyclodextrin, Omega-3-acid ethyl esters and an analogue thereof.

The term “analogue” as used herein refers to a molecule which is structurally similar and/or otherwise exhibit the same functionality as the small molecule of some embodiments of the invention, characterized, in a most preferred embodiment, by their possession of at least one of the abovementioned biological activities of specifically downregulating activity or expression of a component of the lipid synthesis and/or transportation pathways in an astrocyte in the tumor microenvironment as described herein.

According to some embodiments of the invention, the analogue specifically downregulates the activity or expression of a component of the lipid synthesis and/or transportation pathways in an astrocyte in the tumor microenvironment (i.e., a reactive astrocyte) but not in the cancerous cells.

According to some embodiments of the invention, the small molecule is capable of downregulating activity or expression of a component of cholesterol efflux.

According to some embodiments of the invention, the small molecule is an inhibitor of ABCA1 expression or activity.

According to some embodiments of the invention, the small molecule is Probucol or Glyburide, or an analogue thereof.

Probucol (Lorelco) is a strong, BBB-permeable, FDA-approved ABCA1 inhibitor that was also shown to prevent BBB dysfunction and mitigate cognitive and hippocampal synaptic impairments in Alzheimer's models.

Glyburide (Glibenclamide; BIIB093, RP1127) is also a potent ABCA1 inhibitor; however, it doesn't cross well the undisrupted BBB. Glyburide can also inhibit Sulfonylurea receptor 1 (SUR1), a known component of ATP-sensitive potassium channels, and thus serve to significantly reduce edema and brain swelling in different brain injuries ranging from stroke to metastatic brain tumor, which is the main mode-of-actions of Bevacizumab (Avastin), a recently FDA-approved (2009) for the treatment of recurrent or progressive GBM.

Avasimibe (CI-1011) is an inhibitor of sterol O-acyltransferases (SOAT1 and SOAT2, also known as ACAT1 and ACAT2) enzymes, which are involved in the metabolism and catabolism of cholesterol.

Nystatin (Brand Names: Flagystatin, Mycostatin, Nyaderm, Nyamyc, Nystop, Viaderm Kc) is an inhibitor of cholesterol trafficking.

Ezetimibe (Brand Names: Ezetrol, Lypqozet, Nexlizet, Roszet, Vytorin, Zetia) is an inhibitor of cholesterol absorption.

Fenofibrate (Brand names: Antara, Cholib, Fenoglide, Fenomax, Lipidil Supra, Lipofen, Tricor, Triglide)—PPARα—reduces low-density lipoprotein cholesterol (LDL-C), total cholesterol, triglycerides, and apolipoprotein B and increases high-density lipoprotein cholesterol (HDL-C).

2-Hydroxypropyl-β-cyclodextrin (HPPCD) (Brand names:VTS-270, Adrabetadex) is a cholesterol chelator.

Omega-3-acid ethyl esters (Brand names: Lovaza, Omtryg)—affect the regulation of cholesterol uptake and degradation by SREBF1.

Downregulation of an activity or expression of a component of the lipid synthesis and/or transportation pathways in astrocytes of the TME can be effected on the genomic and/or the transcript level using a variety of molecules which interfere with transcription and/or translation (e.g., DNA editing agents, RNA silencing agents, Ribozyme, DNAzyme and antisense), or on the protein level using e.g., antagonists, enzymes that cleave the polypeptide, peptides which interfere with the self-ligation and the like.

According to some embodiments of the invention, the downregulation of activity or expression of a component of the lipid synthesis and/or transportation pathways in an astrocyte in the tumor microenvironment is achieved by RNA silencing.

According to some embodiments of the invention, the agent is an RNA silencing molecule.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

According to some embodiments of the invention, the RNA silencing molecule is selected from the group consisting of an antisense oligonucleotide (ASO), a small interference RNA (siRNA), a microRNA (miRNA), a short hairpin RNA (shRNA), a DNAzyme and a ribozyme.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., RNA of a component of the lipid synthesis and/or transportation pathways in an astrocyte of the TME, e.g., ABCA1) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

The RNA silencing agents of specific embodiments of the present invention include, but are not limited to dsRNA, siRNA, shRNA, miRNA and miRNA mimics.

Accordingly, some embodiments of the invention contemplate use of dsRNA to downregulate protein expression from mRNA. According to one embodiment dsRNA longer than 30 bp are used.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

For example, suitable siRNAs directed against Abca1 can be the shAbca1 set forth by SEQ ID NO: 2.

It will be appreciated that, and as mentioned hereinabove, the RNA silencing agent of some embodiments of the invention need not be limited to those molecules containing only RNA, but further encompasses chemically-modified nucleotides and non-nucleotides.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a component of the lipid synthesis and/or transportation pathways in an astrocyte (e.g., ABca1) can be effected using an antisense polynucleotide capable of specifically hybridizing with an mRNA transcript encoding a component of the lipid synthesis and/or transportation pathways in an astrocyte.

Nucleic acid agents can also operate at the DNA level as summarized infra.

Downregulation of a component of the lipid synthesis and/or transportation pathways in an astrocyte in the TME can also be achieved by inactivating the gene (e.g., ABCA1) via introducing targeted mutations involving loss-of function alterations (e.g. point mutations, deletions and insertions) in the gene structure.

According to some embodiments of the invention, the agent is a DNA editing molecule.

According to some embodiments of the invention, the DNA editing molecule is selected from the group consisting of a meganuclease, a zinc finger nuclease (ZFN), a transcription-activator like effector nuclease (TALEN) and CRISPR/Cas system.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLJDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present invention include sgLuc2 (SEQ ID NO: 4), sgAbca1 (#1) (SEQ ID NO: 5) or sgAbca1 (#2) (SEQ ID NO: 6).

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

According to some embodiments of the invention, the DNA editing molecule is selected from the group consisting of a meganuclease, a zinc finger nuclease (ZFN), a transcription-activator like effector nuclease (TALEN) and CRISPR/Cas system.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

According to some embodiments of the invention, the agent is comprised in a nucleic acid construct under the transcriptional control of a cis acting regulatory element specifically active in the astrocyte of the TME but not in a cancerous cell of the brain tumor.

According to some embodiments of the invention, the cis acting regulatory element is a GBM-associated astrocyte-specific promoter.

According to some embodiments of the invention, the GBM-astrocyte-specific promoter is a glial fibrillary acidic protein (GFAP) promoter.

It should be noted that a functional portion of the GFAP promoter, which is capable of transcribing the GFAP coding sequence in an astrocyte can also be used. A non-limiting example of such a functional portion is the gfaABC1D promoter (set forth by SEQ ID NO: 3).

According to some embodiments of the invention, the glial fibrillary acidic protein (GFAP) promoter comprises SEQ ID NO: 3.

According to some embodiments of the invention, the GBM-associated astrocyte-specific promoter is an ABCA1 promoter such as SEQ ID NO: 8.

According to some embodiments of the invention, the nucleic acid construct is encapsulated in a particle, e.g., for a targeting delivery into the astrocyte in the TME.

According to some embodiments of the invention, the particle is an Adeno-associated virus (AAV) particle.

According to some embodiments of the invention, the agent is conjugated directly or indirectly to a targeting moiety capable of binding to the astrocyte of the tumor microenvironment (e.g., to a reactive astrocyte of the TME) and not to a non-reactive astrocyte or a cancerous cell of the brain tumor.

The targeting moiety can specifically bind to marker(s), structure(s) or epitope(s) which are present on the reactive astrocyte in the TME but which are absent or significantly reduced (e.g., less than 20%, less than 10%) in a cell of the brain tumor.

For example, while reactive GFAP-high expressing astrocytes in the TME express an MHC class I antigenic peptide on the surface, cancerous cells in the brain tumor do not express or exhibit a significantly reduced expression of the MHC class I antigenic peptide of the surface (e.g., at least 50% less expression, e.g., at least 60%, 70%, 80%, 90% less expression).

According to some embodiments of the invention, the targeting moiety is an antibody, an aptamer, a peptide or a particle.

The term “antibody” as used in this invention includes intact molecules as well as functional fragments and/or mimetics thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)2 fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Antibody mimetics are small proteins (usually less than 20 kDa) that mimic CDR display within antibody Fab fragments but lack the Fc. Antibody mimetics can be obtained using methods like phage display. To increase their in vivo stability antibody mimetics can be conjugated to specific sequences by chemical conjugation or genetic fusion (reviewed in Angeline N Ta & Brian R McNaughton. 2017. FUTURE MEDICINAL CHEMISTRY Vol. 9, NO. 12; “Antibody and antibody mimetic immunotherapeutics”; which is fully incorporated herein by reference in its entirety).

Once antibodies are obtained, they may be tested for activity, for example via ELISA.

According to some embodiments of the invention, the antibody is a T cell receptor-like antibody.

According to some embodiments of the invention, the antibody is capable of binding a reactive astrocyte MHC-I complex.

As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

The particle which is used as a targeting moiety to the agent of some embodiments of the invention can be an acellular particle, or a cellular particle.

Non-limiting examples of acellular particles include, viruses or portions thereof, such as Adeno-associated virus (AAV) particle which may carry the agent of some embodiments of the invention (e.g., a polynucleotide) into a cell of interest; and/or a lipid-based particle which is used to enhance fusion of the agent of some embodiments of the invention (e.g., a polynucleotide) with the lipid cell membrane of a cell-of-interest.

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43]. The liposomes may be positively charged, neutral or negatively charged. For Mononuclear Phagocyte System (MPS) uptake, the liposomes can be hydrophobic since hydrophilic masking of the liposome membrane (e.g., by use of polyetheleneglycol-linked lipids and hydrophilic particles) may be less prone to MPS uptake. It is also preferable that the liposomes do not comprise sterically shielded lipids such as ganglioside-GM1 and phosphatidylinositol since these lipids prevent MPS uptake.

The liposomes may be a single lipid layer or may be multilamellar. If the therapeutic agent is hydrophilic, its delivery may be further improved using large unilamellar vesicles because of their greater internal volume. Conversely, if the therapeutic agent is hydrophobic, its delivery may be further improved using multilamellar vesicles. Alternatively, the therapeutic agent (e.g. oligonucleotide) may not be able to penetrate the lipid bilayer and consequently would remain adsorbed to the liposome surface. In this case, increasing the surface area of the liposome may further improve delivery of the therapeutic agent. Suitable liposomes in accordance with the invention are non-toxic liposomes such as, for example, those prepared from phosphatidyl-choline phosphoglycerol, and cholesterol. The diameter of the liposomes used can range from 0.1-1.0 microns. However, other size ranges suitable for phagocytosis by phagocytic cells may also be used. For sizing liposomes, homogenization may be used, which relies on shearing energy to fragment large liposomes into smaller ones. Homogenizers which may be conveniently used include microfluidizers produced by Microfluidics of Boston, MA. In a typical homogenization procedure, liposomes are recirculated through a standard emulsion homogenizer until selected liposomes sizes are observed. The particle size distribution can be monitored by conventional laser beam particle size discrimination. Extrusion of liposomes through a small-pore polycarbonate membrane or an asymmetric ceramic membrane is an effective method for reducing liposome sizes to a relatively well defined size distribution. Typically, the suspension is cycled through the membrane one or more times until the desired liposome size distribution is achieved. The liposomes may be extruded through successively smaller pore membranes to achieve a gradual reduction in liposome size.

According to some embodiments of the invention the cellular particle comprises a whole cell or a portion thereof (e.g., a cell-free particle).

Cell-free particles include, but are not limited to vesicles released or secreted from a cell, e.g., a vesicle including cellular membranes but not a cell nucleus.

As used herein, the term “exosome” refers to an extracellular vesicle that is released from a cell upon fusion of a multivesicular body (MVB) with the plasma membrane. The exosome may (a) have a size of between 30 nm (nanometer) and 120 nm (nanometer) as determined by electron microscopy; (b) comprises a complex of molecular weight >100 kDa (kilodalton), comprising proteins of <100 kDa; (c) comprises a complex of molecular weight >300 kDa, comprising proteins of <300 kDa; (d) comprises a complex of molecular weight >1000 kDa; (e) has a size of between 2 nm and 200 nm, as determined by filtration against a 0.2 μM filter and concentration against a membrane with a molecular weight cut-off of 10 kDa; or (f) a hydrodynamic radius of below 100 nm, as determined by laser diffraction or dynamic light scattering.

Exosomes can also be purified by ultracentrifugation of clarified conditioned media at 100,000×g. They can also be purified by ultracentrifugation into a sucrose cushion. GMP methods for exosome purification from dendritic cells have been described in J Immunol Methods. 2002; 270:211-226.

Exosomes can also be purified by differential filtration, through nylon membrane filters of defined pore size. A first filtration though a large pore size will retain cellular fragments and debris. A subsequent filtration through a smaller pore size will retain exosomes and purify them from smaller size contaminants.

As used herein, an isolated exosome is one which is physically separated from its natural environment. An isolated exosome may be physically separated, in whole or in part, from tissue or cells with which it naturally exists, e.g., stem cells, fibroblasts, and macrophages. In some embodiments of the disclosure, the isolated exosomes may be free of cells or it may be free or substantially free of conditioned media. Typically, the isolated exosomes are provided at a higher concentration than exosomes present in un-manipulated conditioned media.

The particle may be isolated for example by being separated from non-associated components based on any property of the particle. For example, the particle may be isolated based on molecular weight, size, shape, composition or biological activity.

The particle may have a size of greater than 2 nm. The particle may have a size of greater than 5 nm (nanometer), 10 nm, 20 nm, 30 nm, 40 nm or 50 nm. The particle may have a size of greater than 100 nm, such as greater than 150 nm. The particle may have a size of substantially 200 nm or greater.

The particle or particles may have a range of sizes, such as between 2 nm to 20 nm, 2 nm to 50 nm, 2 nm to 100 nm, 2 nm to 150 nm or 2 nm to 200 nm. The particle or particles may have a size between 20 nm to 50 nm, 20 nm to 100 nm, 20 nm to 150 nm or 20 nm to 200 nm. The particle or particles may have a size between 50 nm to 100 nm, 50 nm to 150 nm or 50 nm to 200 nm. The particle or particles may have a size between 100 nm to 150 nm or 100 nm to 200 nm. The particle or particles may have a size between 150 nm to 200 nm.

The size may be determined by various means. In principle, the size may be determined by size fractionation and filtration through a membrane with the relevant size cut-off. The particle size may then be determined by tracking segregation of component proteins with SDS-PAGE or by a biological assay.

The size may comprise a hydrodynamic radius. The hydrodynamic radius of the particle may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the particle may be determined by any suitable means, for example, laser diffraction or dynamic light scattering.

According to some embodiments of the invention, the cell-free particle is comprised in a cell free sample in which the majority of protein is comprised in cell-free particles comprising a plurality of the cell-free particle.

According to some embodiments of the invention, the cell-free particle is derived from a cell selected from the group consisting of a tumor cell, a stem cell, healthy cell, stably transfected cell.

Cellular particles suitable for targeting the agent of some embodiment of the invention include, but are not limited to, cells, such as immune cells capable of crossing the BBB and reaching the tumor microenvironment. Examples include, but are not limited to, innate immune cells (macrophages, monocytes, neutrophils, dendritic cells, innate lymphoid cells, myeloid-derived suppressor cells, and Natural killer (NK) cells) as well as adaptive immune cells (T cells and B cells).

According to some embodiments of the invention, the method further comprising administering to the subject a therapeutically effective amount of a molecule which is associated with lipid uptake by the tumor cells or immune cells in the tumor microenvironment.

According to some embodiments of the invention, the molecule is associated with cholesterol uptake by the tumor cells or the immune cells in the TME.

Molecules which are associated with cholesterol uptake by the tumor cells or immune cells in the TME include, for example, an LDLR antisense/decoy molecule and/or Proprotein convertase subtilisin/kexin type 9 (PCSK9).

PCSK9 (Gene ID: 255738) is a member of the subtilisin-like proprotein convertase family, and it plays a role in cholesterol and fatty acid metabolism.

Without being bound by any theory, the present inventor has envisaged that provision of PCSK9 lowers the expression of LDL receptors in tumor cells or other cells in the TME, which ultimately inhibits uptake of cholesterol by the tumor cells, resulting in starvation of the tumor cells and increasing their susceptibility to apoptosis.

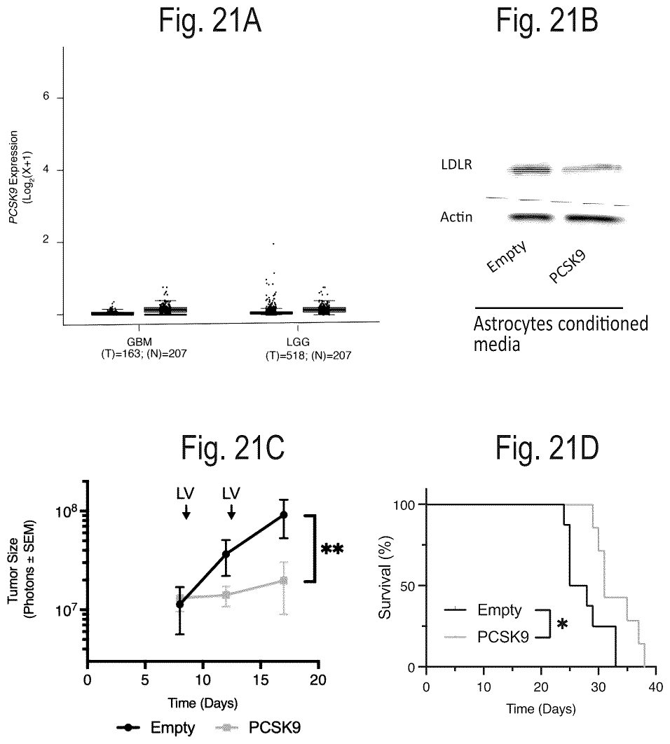

Indeed, Example 11 of the Examples section which follows demonstrates that over-expression of PCSK9 (e.g., using a lentivirus) results in the release (secretion) of PCSK9 to the media (conditioned medium) that, in-turn, lowers the levels of LDLR in glioma cells (FIG. 21B), attenuates GBM growth (FIG. 21C) and prolongs survival of GBM-affected subjected (FIG. 21D).

According to an aspect of some embodiments of the invention, there is provided a method of treating a brain tumor in a subject in need thereof, the method comprising administering to the subject a therapeutically effective amount of a molecule which is associated with lipid uptake by the tumor cells or immune cells in the tumor microenvironment, or a polynucleotide encoding same, thereby treating the brain tumor in the subject.

According to an aspect of some embodiments of the invention, there is provided a therapeutically effective amount of a molecule which is associated with lipid uptake by the tumor cells or immune cells in the tumor microenvironment, or a polynucleotide encoding same, for use in treating a brain tumor in a subject in need thereof.

According to some embodiments of the invention, the molecule which is associated with lipid uptake by the tumor cells or immune cells in the tumor microenvironment, or a polynucleotide encoding same is administered or delivered into the TME.

According to some embodiments of the invention, the molecule which is associated with lipid uptake by the tumor cells or immune cells in the tumor microenvironment is Proprotein convertase subtilisin/kexin type 9 (PCSK9).

It should be noted that since PCSK9 is a secreted protein delivery of a polynucleotide encoding PCSK9 into the TME in the brain can result in the secretion of PCSK9 in the tumor environment, lowering the levels of LDLRs in the tumor cells (e.g., glioma cells) and eventually reducing tumor growth and treating the subject having the brain tumor.

According to some embodiments of the invention, the molecule or the polynucleotide is comprised in or associated with a particle suitable for delivery into a brain of the subject (e.g., into the TME).

According to some embodiments of the invention, the particle is acellular.

According to some embodiments of the invention, the particle is cellular.

According to some embodiments of the invention, the cellular particle is a cell.

According to some embodiments of the invention, the cell is a monocyte.

According to some embodiments of the invention, the cell is an astrocyte.

According to some embodiments of the invention, the acellular particle is a virus comprising a polynucleotide encoding PCSK9.

According to some embodiments of the invention, the acellular particle is a virus comprising a nucleic acid construct comprising a polynucleotide encoding PCSK9.

According to some embodiments of the invention, the acellular particle is an Adeno-associated virus (AAV) particle.

According to an aspect to some embodiments of the invention there is provided a monocyte expressing a heterologous PCSK9 mRNA or protein.

According to an aspect to some embodiments of the invention there is provided an astrocyte expressing a heterologous PCSK9 mRNA or protein.

According to some embodiments of the invention the PCSK9 mRNA which is delivered into the TME is the sequence set forth by GenBank Accession No. NM_174936.4 (SEQ ID NO: 9), which encodes the PCSK9 protein set forth by GenBank Accession No. NP_777596.2 (SEQ ID NO: 10).

It should be noted that some PCSK9 variants may have increased activity, e.g., enhanced degradation of the LDL receptor (LDLRs) at the cell surface compared to the wild type protein, resulting in inhibition of LDL uptake by the tumor cells or immune cells in the TME.

For example, a variant comprising a missense mutation at position 374 of the PCSK9 protein (D374Y) results in a gain-of-function mutation compared to the function of the wild type protein.

According to some embodiments of the invention, the PCSK9 which is expressed in the TME is a PCSK9 variant having increased activity as compared to the wild-type PCSK9 protein (SEQ ID NO: 10).

According to some embodiments of the invention the PCSK9 mRNA which is expressed in the TME (e.g., by a monocyte and/or an astrocyte) is a sequence encoding a gain-of-function mutation resulting in expression of a protein variant having enhanced degradation of the LDL receptor (LDLRs) at the cell surface compared to the wild type protein.

According to some embodiments of the invention the PCSK9 variant comprises the amino acid sequence set forth by SEQ ID NO: 12.

According to some embodiments of the invention the PCSK9 variant is encoded by the nucleic acid sequence set forth by SEQ ID NO: 11.

According to some embodiments of the invention the PCSK9 mRNA is expressed under a regulation of a monocyte-specific promoter or an astrocyte-specific promoter.

For example, suitable monocyte-specific promoter include, but are not limited to the promoter of CD68, or the promoter of allograft inflammatory factor 1 (AIF1).

For example, a suitable astrocyte-specific promoter is a GFAP promoter, e.g., the gfaABC1D promoter (set forth by SEQ ID NO: 3).

According to some embodiments of the invention, endogenous monocytes or astrocytes of the subject are retrieved from a subject in need thereof, transformed ex-vivo to express a nucleic acid construct comprising the coding sequence of PCSK9 under a promoter suitable for expression in monocyte or astrocytes cells, respectively, and then are used for transplantation into the subject.

Methods of preparing monocytes which express a heterologous mRNA or protein are known in the art, e.g., Michael Klichinsky et al., 2020 (Nat. Biotechnol. 38(8):947-953; “Human chimeric antigen receptor macrophages for cancer immunotherapy”, which is fully incorporated herein by reference its entirety).

Methods of preparing astrocytes which express a heterologous mRNA or protein are known in the art, e.g., Rita Perelroizen et al., 2022, Brain. 2022, 145(9):3288-3307. “Astrocyte immunometabolic regulation of the tumour microenvironment drives glioblastoma pathogenicity”; Lior Mayo et al., 2014. Nat Med. 20(10):1147-56. “Regulation of astrocyte activation by glycolipids drives chronic CNS inflammation”; Tom Meyer et al., 2022. Proc Natl Acad Sci USA. 119(35):e2211310119. “NAD⁺ metabolism drives astrocyte proinflammatory reprogramming in central nervous system autoimmunity”; Eloise Hudry, et al., 2019. Neuron. 101(5):839-862. “Therapeutic AAV Gene Transfer to the Nervous System: A Clinical Reality”; and Jarred M Griffin et al., 2019. Gene Ther. 26(5):198-210. “Astrocyte-selective AAV gene therapy through the endogenous GFAP promoter results in robust transduction in the rat spinal cord following injury”; each of which is fully incorporated herein by reference its entirety.

According to an aspect of some embodiments of the invention, there is provided a chimeric polynucleotide comprising a nucleic acid sequence encoding an expression product capable of downregulating an activity or expression of a component of the lipid synthesis and/or transportation pathways, and another heterologous nucleic acid sequence comprising a cis acting regulatory element specifically active in a reactive astrocyte of the microenvironment of the tumor, but not in a cancerous cell of a brain tumor.

As used herein the term “chimeric polynucleotide” refers to a polynucleotide which comprises at least two distinct nucleic acid sequences, wherein the combination of same in the chimeric polynucleotide is not found in nature.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to some embodiments of the invention, the chimeric polynucleotide of some embodiments of the invention is an isolated polynucleotide.

The term “isolated” refers to at least partially separated from the natural environment e.g., from a human cell.

According to some embodiments of the invention, the cis acting regulatory element is a promoter, e.g., as described above.

According to an aspect of some embodiments of the invention, there is provided a composition of matter comprising the chimeric polynucleotide of some embodiments of the invention and a particle encapsulating or attached to the chimeric polynucleotide.

According to some embodiments of the invention, the particle is an Adeno-associated virus (AAV) particle.

According to an aspect of some embodiments of the invention, there is provided an article of manufacture comprising a small molecule capable of downregulating an activity or expression of a component of the lipid synthesis and/or transportation pathways, the small molecule being conjugated to an antibody or fragment thereof capable of binding an astrocyte in the tumor microenvironment (a reactive astrocyte) and not to a non-reactive astrocyte or a cancerous cell of the brain tumor.

As used herein, the term “treating” includes abrogating, substantially inhibiting, slowing or reversing the progression of a pathology (e.g., brain tumor), causing the reduction, remission, or regression of a pathology, substantially ameliorating clinical or aesthetical symptoms of a pathology or substantially preventing the appearance of clinical or aesthetical symptoms of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

According to some embodiments of the invention, treating comprises reducing the size of a brain tumor (e.g., glioblastoma) following a pre-determined time period of administering to the subject the therapeutically effective amount of the agent of some embodiments of the invention as compared to the size of the brain tumor prior to administering the therapeutically effective amount of the agent.

It should be noted that the size of the brain tumor can be determined using various imaging techniques such as magnetic resonance imaging (MRI) scan, functional MRI (fMRI), computed tomography (CT) scan, positron emission tomography and/or a computed tomography (PET-CT) scan. In addition, depending on the tumor type, inhibition of tumor growth can be also determined using blood levels of cancer-specific markers.

According to some embodiments of the invention, treating comprises increasing the survival of the subject having the brain tumor as compared to the survival of the subject when receiving conventional treatment without the use of the agent of some embodiments of the invention.

While the agent of some embodiments of the invention is useful for treating the subject having the brain tumor, the method of some embodiments of the invention also encompasses a combinational therapy.

For example, the patient having the brain tumor can be subjected to a surgery to remove the brain tumor, with or without chemotherapy, radiation therapy, and/or anti-angiogenesis therapy (e.g., using an anti VEGF antibody) along with administration of the agent of some embodiments of the invention.

It should be noted that in some cases the brain tumor cannot be removed using a surgery, and then the subject having the tumor can receive chemotherapy, radiation therapy, and/or anti-angiogenesis therapy (e.g., using an anti VEGF antibody) along with administration of the agent of some embodiments of the invention.

For example, in the case of GBM, a conventional treatment includes a brain surgery (e.g., resection of the brain tumor), chemotherapy, radiation therapy and/or targeted therapy (anti-angiogenesis).

The conventional (standard) chemotherapy for GBM includes temozolomide (TMZ), which is usually administered in combination with radiation therapy, e.g., every day during radiation therapy and then for six cycles after radiation during the maintenance phase. Additional drugs which are commonly used upon tumor progression include Lomustine (chemotherapy) and bevacizumab (“AVASTIN”; targeted therapy, e.g., anti VEGF antibody).

According to some embodiments of the invention, the method further comprising administering to the subject chemotherapy.

According to some embodiments of the invention, the chemotherapy is temozolomide (TMZ).

According to some embodiments of the invention, the method further comprising administering radiation therapy to the subject.

According to some embodiments of the invention, the method further comprising administering to the subject an anti VEGF antibody.

According to some embodiments of the invention, treating comprises reprograming the tumor chemoresistance to therapy (TMZ). This is important in case of both primary and postoperative GBM recurrence.

According to some embodiments of the invention, treating comprises subjecting the subject having the brain tumor to a combinational therapy, e.g., administering the agent to the subject along with additional chemotherapy, anti-angiogenesis therapy, and/or radiation therapy.

According to some embodiments of the invention, the agent is administered to the subject prior to, along with or after removal of the brain tumor (or at least part thereof) by a surgery.

The agent which is capable of downregulating activity or expression of a component of the lipid synthesis and/or transportation pathways in an astrocyte in the tumor microenvironment or the molecule which is associated with lipid uptake by the tumor cells or immune cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent which is capable of downregulating activity or expression of a component of the lipid synthesis and/or transportation pathways in an astrocyte in the tumor microenvironment and/or the molecule which is associated with lipid uptake by the tumor cells or immune cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the agent which is capable of downregulating activity or expression of a component of the lipid synthesis and/or transportation pathways in an astrocyte in the tumor microenvironment and/or the molecule which is associated with lipid uptake by the tumor cells or immune cells) effective to prevent, alleviate or ameliorate symptoms of a disorder (e.g., brain tumor, e.g., GBM) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide astrocyte's (e.g., reactive astrocytes) levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As described, the agent can be comprised in a nucleic acid construct under the transcriptional control of a cis acting regulatory element specifically active in the cell or interest, depending on the type of agent used. For example, a cis-acting regulatory element active in an astrocyte of the tumor microenvironment, in tumor cells or in immune cells in the tumor microenvironment.

The nucleic acid construct is preferably suitable for mammalian cell expression. Such a nucleic acid construct includes a promoter sequence for directing transcription of polynucleotide sequence of interest in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a Y LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as GFAP that is astrocyte specific (e.g., gfaABC1D, SEQ ID NO: 3), albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

The vector may or may not include a eukaryotic replicon.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the polynucleotide of interest since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

### Animals

C57BL/6J, Gfap-TK (B6.Cg-Tg(Gfap-TK)7.1Mvs/J), Gfap-CRE (B6.Cg-Tg(Gfap-cre)77.6Mvs/2J, stock no 024098), iDTR (Gt(ROSA)26Sor^{tm1(HBEGF)Awai}) and RiboTag (B6N.129-Rpl22tm1.1Psam/J) mice were purchased from Jackson Laboratory (ME, USA). Female Gfap-TK^{+/-} mice were crossed with male C57BL/6J to generate Gfap-TK^{+/-} or WT mice. Female Gfap-Cre^{+/-} were crossed with male RiboTag^{fl/fl} or iDTR^{fl/fl} to generate F1 littermates of Gfap-Cre^{+/-} RiboTag^{fl/-} or Gfap-Cre^{+/-}-iDTR^{fl/-} or Gfap-Cre^{-/-}-iDTR^{fl/-} mice. All animals were kept in a pathogen-free facility at the Tel Aviv University Faculty of Medicine and were housed five animals per cage under a standard light cycle (12 hours:12 hours light:dark) with ad libitum access to water and food. All experiments were carried out in accordance with guidelines prescribed by the Institutional Animal Care and Use Committee of Tel Aviv University.

### Cell Lines

GL261-Luc2 cells (#9361, Caliper), CT-2A-Luc2 (#SCC195, Merck LTD), U87EGFRvIII (described in Villa, G. R., et al. Cancer Cell (2016) 30: 683-693), and 293T AAVpro cells (#632273, Takara) were grown in DMEM (Gibco #41965-039) with 10% FBS (Gibco #12657-029) and 1% Pen-Strep (Gibco #15140-122). 293T medium was further supplemented with non-essential amino acids (BI, #01-340-1B), and 1% sodium pyruvate (BI, #03-042-1B). Cells were maintained free of Mycoplasma contamination; routine Mycoplasma analysis was performed using EZ-PCR™ Mycoplasma Detection Kit (BI #2-700-20). In Lipoprotein starvation experiments, normal FCS was replaced with lipoprotein-deficient serum (LPDS, Sigma Aldrich, #S5394).

### Primary Glial Cultures

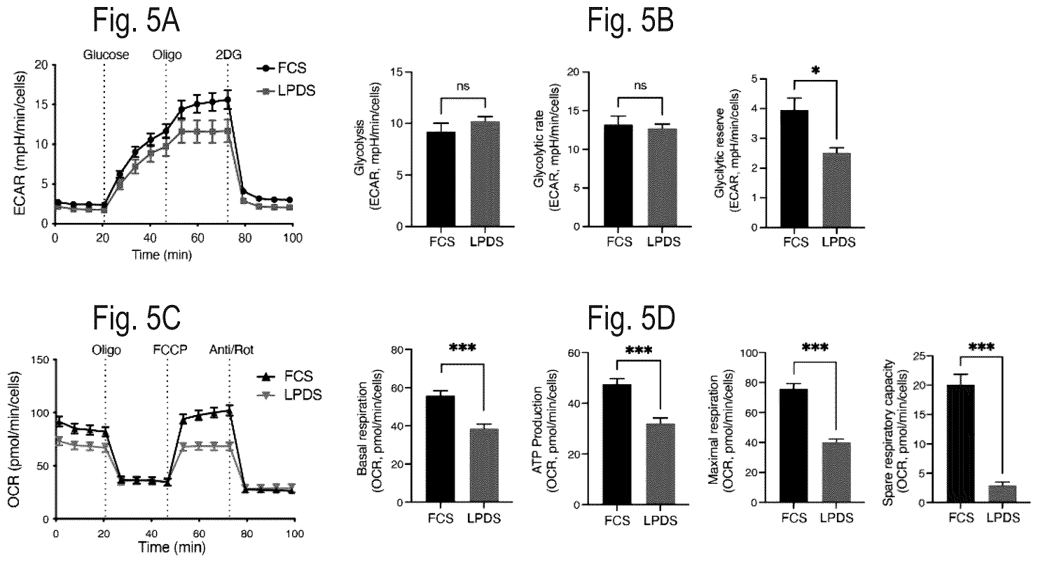

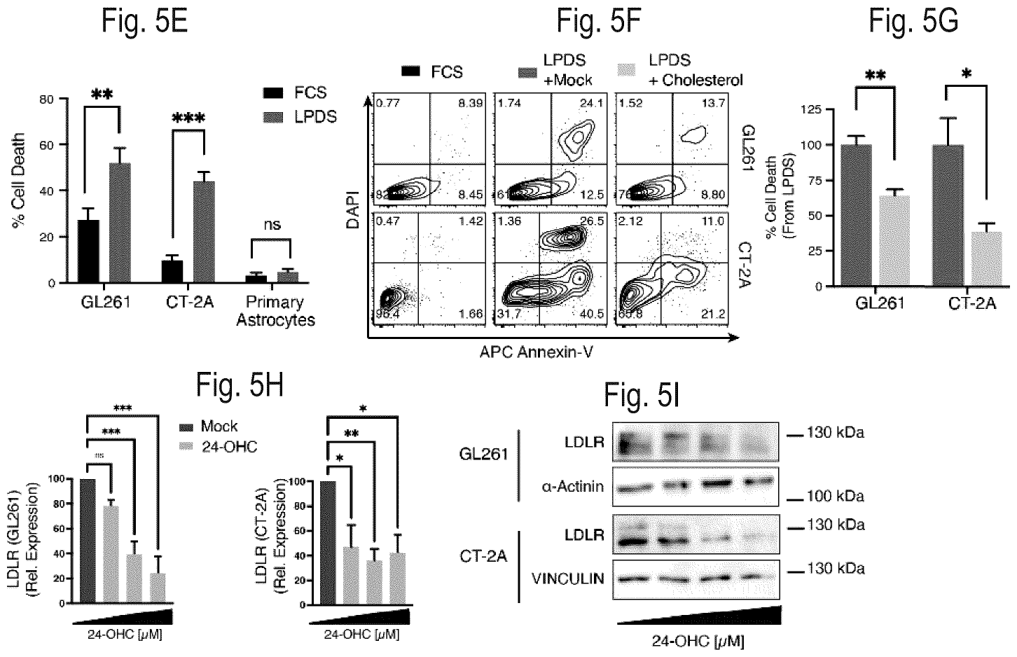

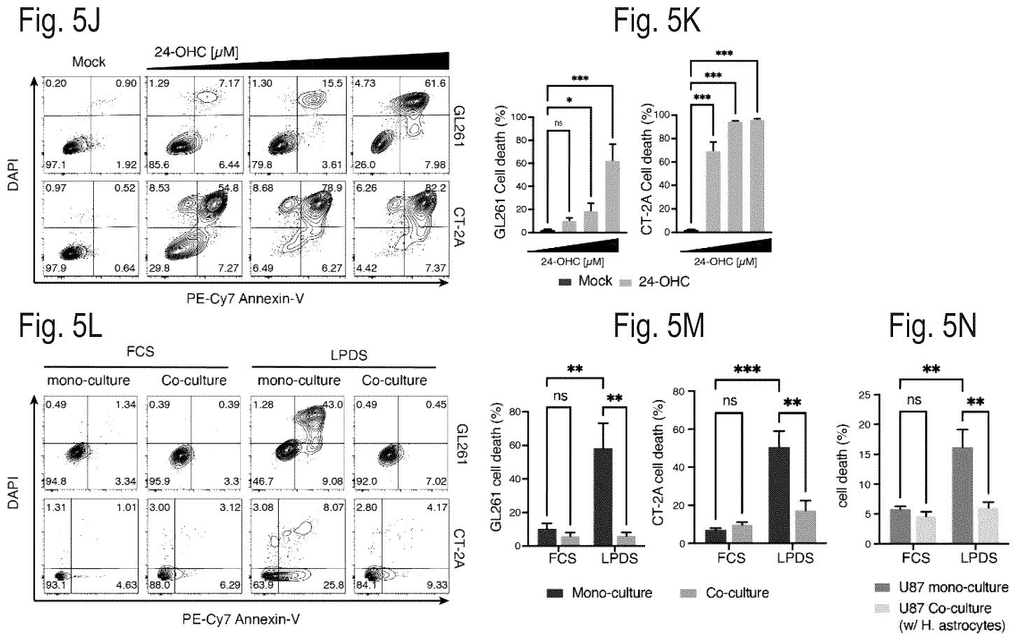

Primary murine mixed glia, astrocytes, and microglial cultures were prepared as previously described (Mayo, L., et al. Nat Med (2014) 20: 1147-1156). Primary astrocytes cultures were found to be >99% GFAP+ positive by immunofluorescent staining (Supplementary FIG. 5E). Microglial cultures were isolated by subjecting confluent mixed glial cultures to mild trypsinization (0.05% Trypsin in DMEM) according to previously published protocols27. This results in the detachment of an intact layer of cells containing virtually all the astrocytes, leaving undisturbed a population of firmly attached cells. Primary microglial cultures were identified as >99% IBA1+ by immunofluorescent staining, and ˜97.8% positive for the microglial marker CD11b (1:50; M1/70, Biolegend #101251) and negative for the astrocyte marker GLAST (1:11, ACSA-1, Miltenyi Biotec, #130-095-821) by FACS analysis (Supplementary FIGS. 5E,F). The following reagents were used for analysis: anti-IBA1 (1:500, Wako Chem, #019-19741), anti-GFAP (1:500, 4A11, 1B4, 2E1, BD Pharmingen #556330), anti-CD11b (1:50; M1/70, Biolegend #101251), and anti-GLAST (1:11, ACSA-1,Miltenyi Biotec, #130-095-821). For microglial proliferation analysis, pure microglial cultures were stained with CellTrace™ Violet Cell Proliferation Kit (Invitrogen, #C34517), according to the manufacturer's instructions. Human astrocytes (ScienceCell, #1800) were grown according to the manufacturer's instructions. In some studies, cells were treated with 2 mM N-Nitro-L-arginine methyl ester hydrochloride (L-NAME, Sigma, N5751), or with 20 μg/ml anti-mouse CSF1R mAb (BioXcell, clone AFS98, #BE0213), anti-mouse TGFb mAb (BioXcell, clone 1D11.16.8, #BE0057), anti-mouse IL10R mAb (BioXcell, clone 1B1.3A, #BP0050), anti-mouse IFNg mAb (Biolegend, clone XMG1.2, 505834), anti-mouse IL6R mAb (BioXcell, clone 15A7, #BE0047), or appropriate isotype control IgG (BioXcell, #BE0090, BE0090). All mAbs were used at a final concentration of 25 μg/ml.

### Preparation of Glioblastoma-Conditioned Medium (GBM-CM)

A total of 2×10⁶ GL261 cells (a murine glioma model cell line) were cultivated in a 10 cm culture plate for 48 hours, media was removed and 8 mL of fresh medium, containing DMEM with 1% FCS and 1% Pen-Strep was added. The supernatant was collected after 24 hours, centrifuged at 500 g. for 10 min at 4° C., sterile filtered using a 22-μm filter and stored at −80° C. For microglial stimulation, GBM-CM was diluted 2:1 with DMEM.

### Preparation of Astrocytes-Conditioned Medium (ACM)

A total of 5×10⁶ primary astrocytes were cultivated in a 10 cm culture plate, and stimulated for 12 hours with DMEM containing 1% FCS and 1% Pen-Strep (to generate ACM) or GBM-CM (to generate T-ACM). Media was removed, and 8 mL of fresh medium, containing DMEM with 0.5% BSA (Millipore, #810683) and 10 mM Hepes, was added. The supernatant was collected after 24 hours, centrifuged at 500 g for 10 min at 4° C., sterile filtered using a 22-am filter and stored at −80° C.

### Chemotaxis Assay

Spleen monocytes (CD11b⁺/CD3⁻/CD45R⁻/CD117⁻/Ly-6G⁻/NK1.1⁻/Siglec F⁻/SSC^{low}) were isolated from C57BL/6 mice using EasySep Mouse Monocyte Isolation Kit (Stemcell, #19861). Monocytes were stained with CellTrace™ CFSE Cell Proliferation Kit (Thermo Fisher Scientific, #C34554), and a total of 2×10³ monocytes per well were plated in IncuCyte® ClearView 96 well Cell Migration Plate (Sartorius, #4582), which was pre-coated with 50 g/ml Matrigel® (Corning, #FAL356237), and stained with CellTrace™ CFSE Cell Proliferation Kit (Thermo Fisher Scientific, #C34554). ACM or T-ACM were added to the lower chamber. In some experiments, the T-ACM was supplemented with 30 g/ml of anti-mouse CCL2 neutralizing mAb (BioXcell, clone 2H5, #BE0185) or isotype control (BioXcell, #BE0091). Chemotaxis was measured following 2 hour incubation at 37° C. as the percentage of monocytes that infiltrated the lower chamber using the IncuCyte® ZOOM instrument and analyzed with IncuCyte® ZOOM software v2020C.

### Tumor Model and Treatments

For intracranial mouse glioma, mice were anesthetized, positioned in a Stereotaxic Alignment System, and injected with 1.5×10⁴ GL261 or CT-2A cells in 2 μl of DMEM. Injections were made to the right frontal lobe, approximately 2.5 mm lateral and 0.1 mm caudal from bregma at a depth of 3 mm. In-vivo bioluminescence imaging, following XenoLight D-luciferin Potassium Salt administration (150 mg/kg) i.p. administration, was determined using IVIS Spectrum system (PerkinElmer). Ganciclovir (GCV, Cymevene, Roche #SAP-10051872; 25 mg/kg), Diphtheria-toxin (DT, Sigma-Aldrich, #D0564; 1100 ng/mice), or vehicle control (PBS) were administered daily following tumor establishment (day 10), as previously described (Mayo, L., et al. Nat Med (2014) 20: 1147-1156). CD8⁺ T cell depletion was performed using an anti-CD8 monoclonal antibody (53.6-7, Bioxcell) or an isotype control monoclonal antibody (2A3, Bioxcell), as previously described (Takenaka, M. C. et al. Control of tumor-associated macrophages and T cells in glioblastoma via AHR and CD39. Nat Neurosci 22, 729-740 (2019)). GBM-bearing mice were intraperitoneally injected (0.1 mg/mouse) with an anti-CD8 or an isotype control mAbs, 1 day before, and 7 days after, GCV administration. CD8 T-cell depletion was validated by FACS analysis using a Sony SH800 FACS instrument (Sony Biotechnology). Data analysis was performed using FlowJo v10 (TreeStar, USA). The following reagents were used for analysis: anti-CD45 (1:100; 30-F11, Biolegend, #103106), anti-CD3□ (1:50; 145-2C11, Biolegend #100335), and anti-CD8a (1:100; 53-6.7, Biolegend, #100765).

### Immunofluorescence

Animals were perfused with ice-cold PBS, followed by 4% paraformaldehyde in 0.1 M PBS. Tissues were cryoprotected in 0.1 M PBS plus 30% sucrose, and cut with cryostat into 10-m-thick sections. Sections were blocked in 5% goat serum and 5% donkey serum containing 0.3% Triton™ X-100 (Sigma-Aldrich, #9002-93-1) and 0.3 M Glycine (Holland Moran, #BP381-1), and incubated overnight at 4° C. with following antibodies: GFAP (chicken, 1:1000, Abcam, #ab4674), IBA1 (rabbit, 1:1000, Wako Chem, #019-19741), HA (rat, 1:300, Roche, #11-867-423-001), MBP (chicken, 1:50, Chemicon #AB9348), NeuN (mouse, 1:100, EMD Millipore, #MAB377) DRAQ7 (1:500 Biolegend, #424001), Cleaved Caspase 3 (rabbit, 1:500, Cell Signaling Technology, #9664S), and ABCA1 (rabbit, 1:200, Novusbio, #NB400-105), Annexin A2 (rabbit, 1:500, proteintech, #11256-1-AP), Ki67 (Rat, 1:500, Thermo Fisher Scientific, #14-6698-82), PD-L1 (Rat, 1:500, BioXcell, #BE0101), or CD74 (Rabbit. Clone EPR25399-94, 1:500, Abcam, #ab289885). The next day sections were washed 3 times, and incubated with an appropriate fluorophore-conjugated goat secondary Abs (1:500) from Abcam (#ab150167 or ab150176) or Thermo Fisher scientific (#A11037, A11020, or A32733) for 1 hour at room temperature. Sections were mounted in Prolong™ Gold containing DAPI (Invitrogen, P36935). Appropriate isotype control antibodies were used to control for nonspecific binding. All settings were kept the same during image acquisition of comparable images including magnification, laser intensity, and optical configuration.

### Image Acquisition

Images were acquired using a Leica SP8 confocal microscope and Leica LAS AF software and processed using Fiji and LAS X. All settings were kept the same.

### Analysis of TAMs

GL261-implanted mice were sacrificed 17 days after implantation, and single-cell suspension was prepared as previously described (Mayo, L., et al. Nat Med (2014) 20: 1147-1156). In brief, mice were anesthetized and perfused with 10 ml of PBS, their brains were isolated, enzymatically dissociated using Collagenase type III (Worthington Biochemical, #LS004182), and Dispase II (Roche, #04942078001), and mechanically dissociated using gentleMACS™ Dissociator. Myelin was removed by resuspending the homogenate in 25% Percoll solution, underlaid by 75% Percoll and overlaid with 5 mL Hanks' Balanced Salt solution (HBSS). Centrifugation at 1,000 g for 30 minutes with slow acceleration and without breaks created a gradient that separated the cell pellet on the bottom from the myelin, which was carefully aspirated. Cells were carefully collected from the 75%-25% interphase. TAMs (CD11b⁺CD45⁺) or microglial cells (CD11b⁺CD45^{dim}) were analyzed or sorted into TRIzol™ LS reagent (Invitrogen, #10296028) for RNA purification using Sony SH800 Fluorescence-activated cell sorting (FACS) (Sony Biotechnology). Data analysis was performed using FlowJo™ v10 (TreeStar, USA). The following reagents were used for analysis: anti-CD11b (1:50; M1/70, eBioscience, #12-0112-82, Biolegend #101251), anti-CD45 (1:100; 30-F11, Biolegend, #103116), anti-PD-L1 (1:50; 10F.9G2, Biolegend, #124314).

### Filipin II Staining

Filipin III (Cayman Chemical Company, #70440-1) was used to evaluate cholesterol content in the cells using FACS and immunofluorescence staining. Filipin III (Cayman Chemical Company, #70440) staining was performed at 100 μg/mL for 25 minutes at room temperature, as previously described (Ma, X. et al. Cholesterol Induces CD8(+) T Cell Exhaustion in the Tumor Microenvironment. Cell Metab 30, 143-156 e5 (2019)). For FACS analysis cells were first fixated with 0.5% in Paraformaldehyde (Electron Microscopy Sciences, #15710; dissolved in PBS), stained, and then analyzed by CytoFLEX LX flow cytometer (Beckman Coulter) equipped with a 355 nm (UV) laser. Staining was performed at 100 μg/mL for 25 minutes at room temperature. For FACS analysis cells were first fixated with 0.5% in Paraformaldehyde.

### Metabolic Flux Assays

Real-time extracellular acidification rate (ECAR) and oxygen consumption rate (OCR) measurements were made with an XF-96 Extracellular Flux Analyzer (Seahorse Bioscience). 1×10⁵ cells were plated into each well of Seahorse X96 cell culture microplates and preincubated at 37° C. for 24 hours in 5% CO₂ in either full serum (FCS) or lipoprotein-deprived serum (LPDS) supplemented media. The sensor cartridge for the Xfe96 analyzer was hydrated in a 37° C. non-CO₂ incubator a day before the experiment. OCR and ECAR were measured under basal conditions and after the addition of the following compounds: 1.5 M oligomycin, 2 M FCCP (carbonyl cyanide4-(trifluoromethoxy)phenylhydrazone), 0.5 M rotenone and 0.5 M antimycin, 10 mM glucose, and 50 mM 2-deoxy-d-glucose (all obtained from Sigma) as indicated. Data were expressed as the rate of oxygen consumption in pmol/min or the rate of extracellular acidification in mpH/min, normalized to DNA labeling in individual wells determined by the Hoechst 33342 staining. Results were collected with Wave software version 2.4 (Agilent).

### Cell Viability Assay

GL261, CT-2A, or astrocytes were seeded at a density of 1.5×10⁵ cells per well in a 6-well plate. Cells were treated for 72 hour FCS- or LPDS-supplemented media for 5 days. In some studies, cells were treated with Lovastatin (Thermo Scientific, #PH1285R) at 2.5 or 5 M (GL261, CT-2A, astrocytes), 24(s)-hydroxy-cholesterol (Cayman Chemical Company, #10009931) at 2.5, 5 or 10 M (GL261 cells) or at 1.25, 2.5, 5 M (CT-2A cells) for 72 hours, or 250 ng/ml cholesterol (Sigma-Aldrich, #C4951). Cell death was analyzed by FACS analysis based using Annexin-V assay according to the manufacturer's instructions (Biolegend, #640951, 640941). Microglial cell death was determined by LDH Assay (Sigma-Aldrich, #4744934001) according to the manufacturer's instructions.

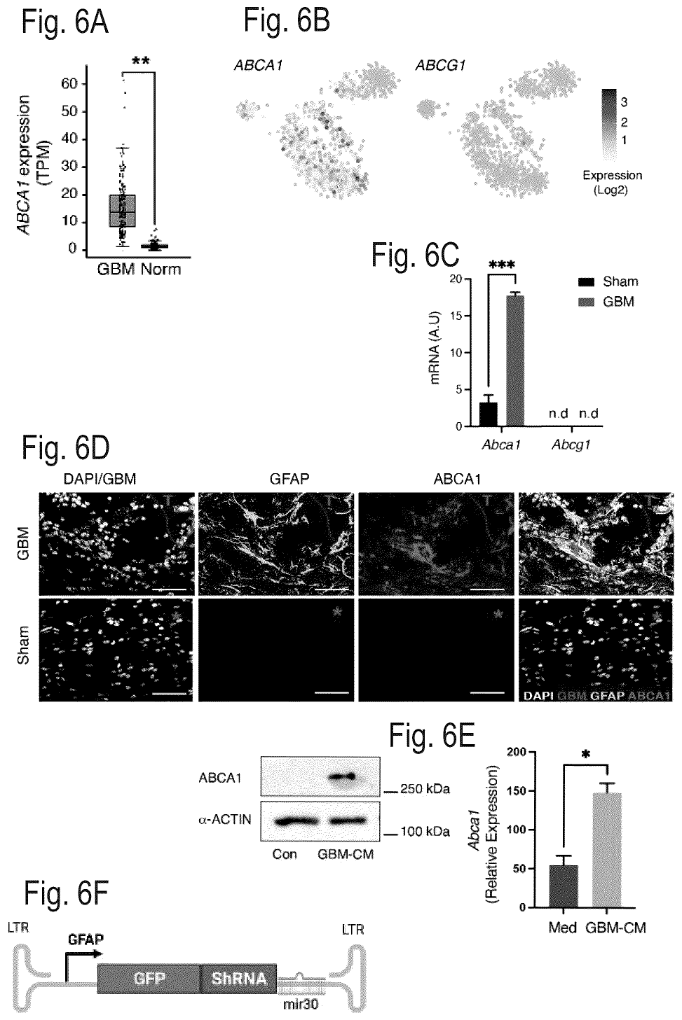

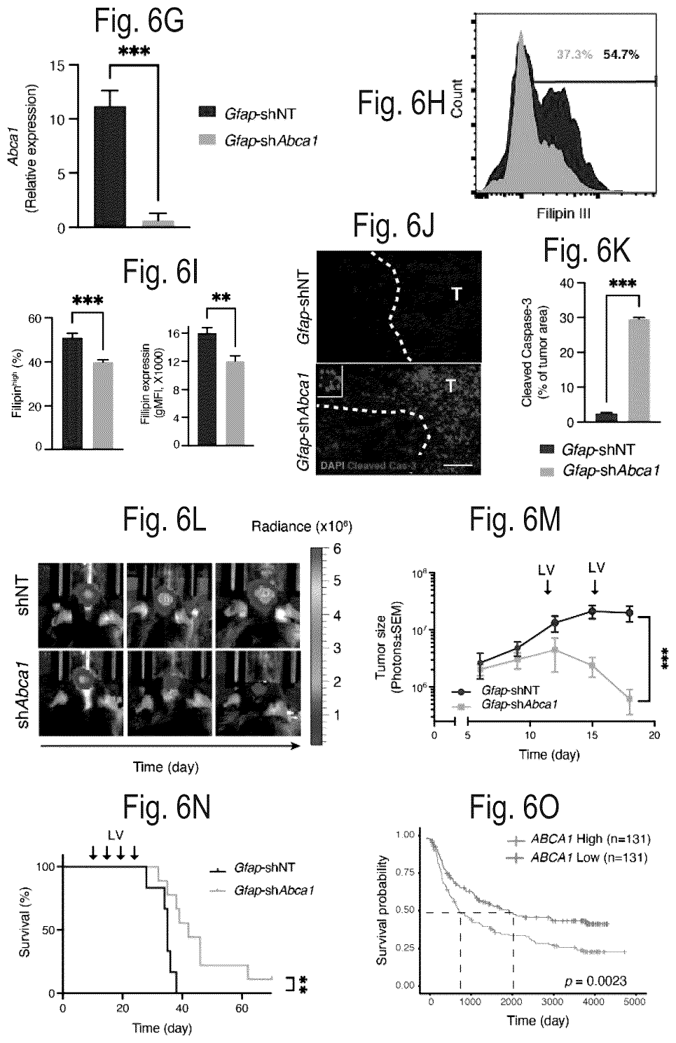

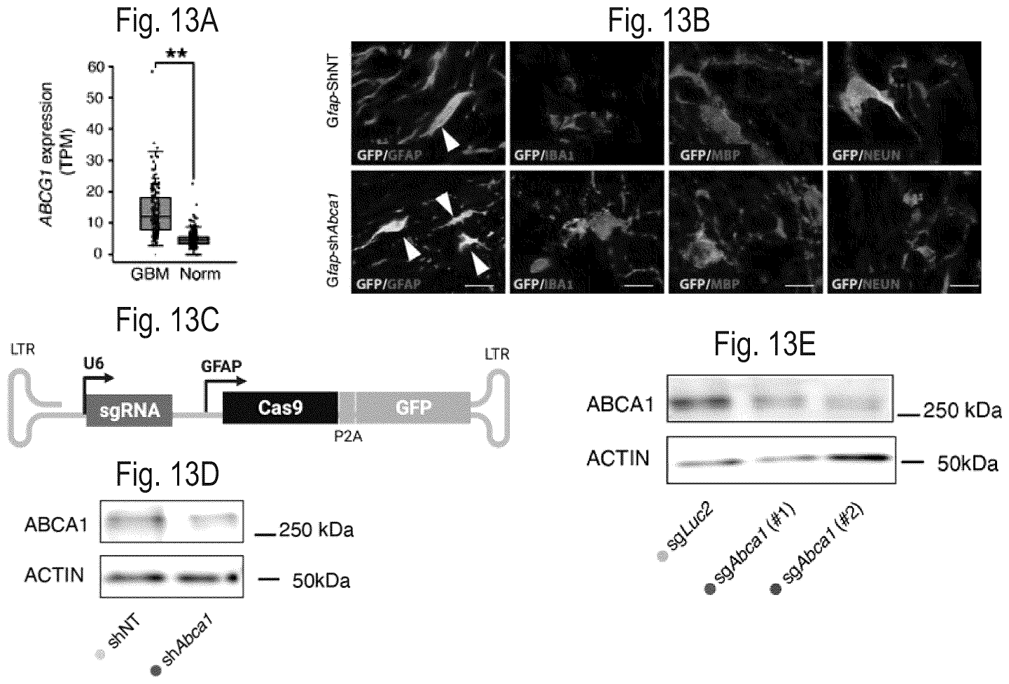

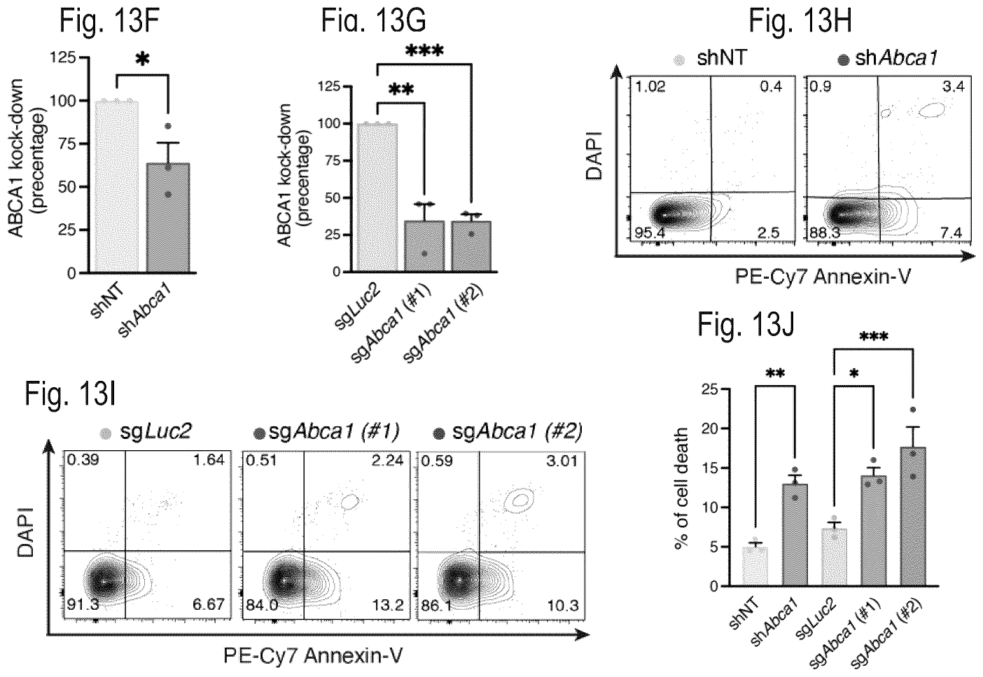

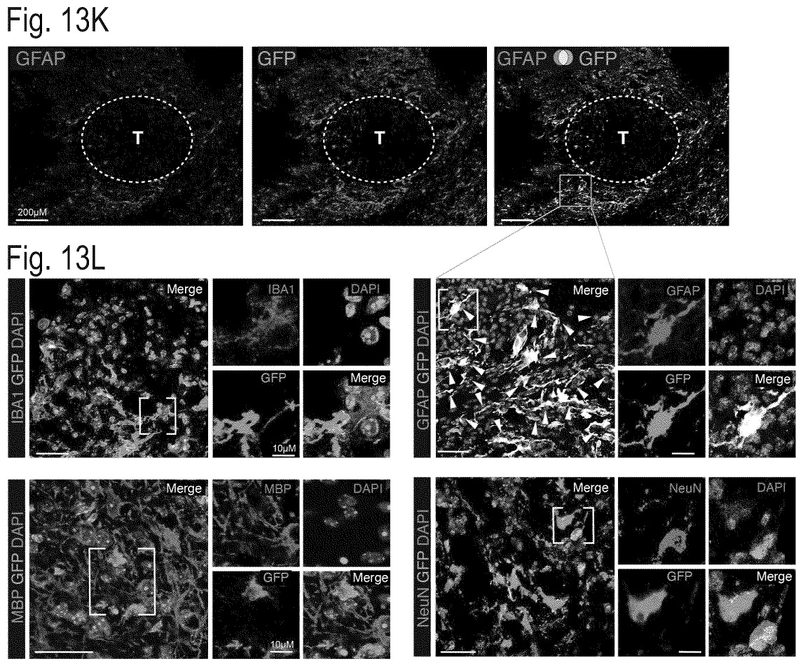

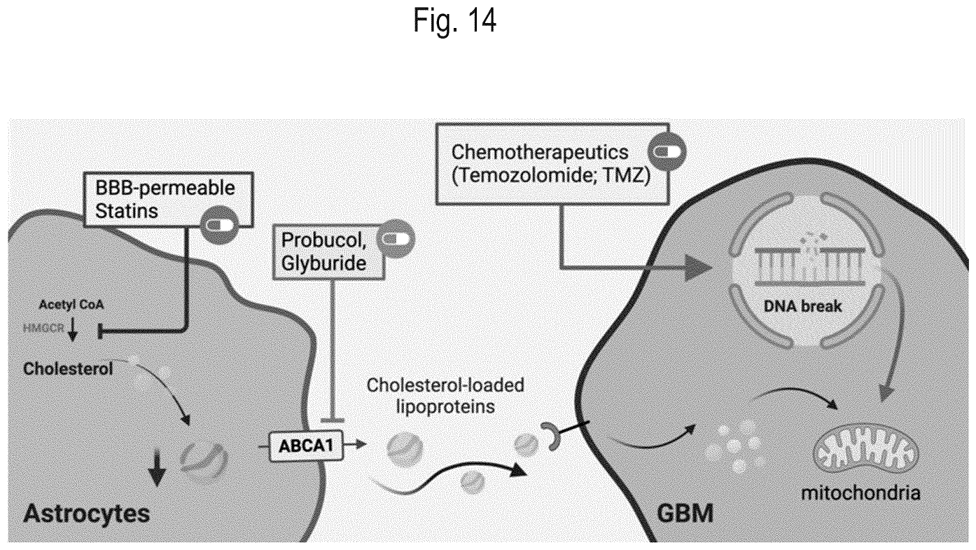

In vivo astrocyte-specific knock-down with shRNA Lentivirus pLenti-GFAP-EGFP-mir30-sh Abca1 (FIG. 6F) harboring shRNA sequences against Abca1 was cloned into the pLenti-GFAP-EGFP-mir30-shB4galt6 vector backbone (Mayo, L. et al. Regulation of astrocyte activation by glycolipids drives chronic CNS inflammation. Nat Med 20, 1147-56 (2014)) and replaced by validated Abca1-targeting shRNA sequence (MISSION® TRC shRNA clone #TRCN0000271860, Sigma Aldrich), as was previously described (Mayo et al., 2014, Supra). Non-targeting shRNA vector (pLenti-GFAP-EGFP-mir30-shNT) was previously described (Mayo et al., 2014, Supra). pLenti-GFAP-CRISPRv2GFP-sgRNA vectors (FIG. 13C), were generated by cloning the relevant single-guide RNA (sgRNA) to pLentiCRISPRv2GFP (Addgene #82416) as previously described (Walter, D. M. et al. Systematic In Vivo Inactivation of Chromatin-Regulating Enzymes Identifies Setd2 as a Potent Tumor Suppressor in Lung Adenocarcinoma. Cancer Res 77, 1719-1729 (2017)). The EF-1a promoter, which drives the expression of the polyprotein Cas9-P2A-GFP, was then replaced with the gfaABC1D promoter (GeneArt, Thermo Fisher Scientific) using XbaI and EcoRI restriction enzymes (#R3101 and #R0145, New England Biolabs). CRISPR-Cas9 sgRNA sequences were designed using the Broad Institute's sgRNA GPP Web Portal (www(dot)portals(dot)broadinstitute(dot)org/gpp/public/analysis-tools/sgrna-design). All sequences are detailed in Table 3. Lentivirus particles were then generated by transfecting 3.9×10⁶ 293T cells using 12.1 μg PEI MAX (Polysciences, #24765-1) with 6.1 μg pLenti-GFAP-EGFP-mir30-shRNA or pLenti-GFAP-CRISPRv2GFP-sgRNA vectors with the packing plasmids [4.5 μg psPAX2 (Addgene, #12260) and 1.5 μg pMD2.G (Addgene, #12259)]. 48 hours after transfection supernatant was concentrated using Lenti-X Concentrator (Takara, #631232) and stored at −80° C. until use. The viral titrate was determined using the qPCR Lentivirus titration kit (ABM, #LV900) according to the manufacturer's instructions.

### Immunoblotting

Cells were lysed in RIPA buffer supplemented with protease inhibitors (Cell Signaling). A total of 35 μg of sample was separated by 7.5, 10, or 12% Tris-Glycine gels, transferred to nitrocellulose membranes (Millipore), and developed with the following antibodies: anti-ABCA1 (rabbit, 1:1000; Novus Biologicals, #NB400-105), anti-LDLR (rabbit, 1:500, ProteinTech, #10785-1-AP), anti-ACTIN (mouse, 1:1,000-30,000; MP Biomedicals, #08691001), anti-a-ACTININ (mouse, 1:1000; Cell Signaling Technology, #69758S) and anti-VINCULIN (rabbit, 1:1000, Proteintech, #26520-1-AP). Blots were developed using a Clarity™ Western ECL kit (Bio-Rad, #1705061) on Amersham Imager 600 (GE Healthcare). Expression levels were normalized to ACTIN, a-ACTININ, or VINCULIN. Quantification was done using Image Studio Lite software version 5.2 (LI-COR Biosciences).

### Metabolic Pathway Analysis

iMAT (Zur, H. et al., Bioinformatics (2010) 26: 3140-3142) was employed to incorporate gene expression levels into the metabolic model to predict a set of high and low activity reactions. Network integration was determined by mapping the genes to the reactions according to the metabolic model, and by solving a constraint-based modeling (CBM) optimization to find a steady-state metabolic flux distribution (Bordbar, A. et al., Nat Rev Genet (2014) 15, 107-120). By using the CBM approach, permissible flux ranges were assigned to all the reactions in the network, in a way that satisfies the stoichiometric and thermodynamic constraints embedded in the model and maximizes the number of reactions whose activity is consistent with their expression state. The pathway enrichment analysis was carried by a hypergeometric test where the background is the number of reactions found in the human model, and the overlap of each metabolic pathway with the set of active ((top 20%) and inactive reactions (low 20%) were then examined via hypergeometric test.

nCounter Gene Expression

Total RNA (100 ng) was analyzed using the nCounter® Mouse Immunology V1 Panel according to the manufacturer's instructions (NanoString Technologies). Data were analyzed using nSolver™ Analysis software. Functional enrichment analysis was performed using the Expander (Ulitsky, I., et al. Nat Protoc (2010) 5: 303-322) and g:Profiler (Raudvere, U., et al. Nucleic Acids Res (2019) 47: W191-W198) platforms.

### RiboTag

Sham or Tumor-bearing mice were sacrificed and were subjected to perfusion through the left ventricle with ice-cold PBS, following by perfusion with 10 mL ice-cold 1% PFA (paraformaldehyde) (Electron Microscopy Sciences, #15710; dissolved PBS). Brains were harvested, and the right hemisphere was homogenized as previously described (Sanz, E., et al. Proc Natl Acad Sci USA (2009) 106: 13939-13944). Briefly, samples were homogenized using 7 mL dounce homogenizer in 10% w/v supplemented homogenization buffer. Homogenate was transferred to Eppendorf® tube and centrifuges for 15 minutes at 15,000 g at 4° C. Supernatant was transferred to a new tube and 5 μl of mouse monoclonal anti-HA antibody was added (Biolegend, #901515). Samples were placed on a rotator for 6 hours at 4° C. 200 μl of Pierce™ beads (Pierce, #88803) or 70 μl of Dynabeads (Invitrogen, #10004D) were washed with 800 μL homogenization buffer, and coupled with the tissue-antibody homogenate for an overnight rotation at 4° C. The following day, samples were placed on a magnet, and supernatant was removed before washing the pellets three times for 10 minutes in high salt buffer. 350 μl RLT Lysis Buffer were added to the beads and RNA was extracted using RNeasy Mini kit (Qiagen, #74104) per manufacturer's instructions and quantified with a Qubit RNA HS Assay Kit (ThermoFischer, #Q32852). RNA was kept in −80° C. until usage.

### RNA Sequencing and Processing

RNA extracted from immunoprecipitated polyribosomes was used to prepare libraries by NebNext® rRNA depletion kit (#E6310), NEBNext® Ultra™ II RNA Library Prep Kit for Illumina® (#E7770G), and NEBNext® Multiplex Oligos for Illumina® (#E7335G) kit, according to manufacturers' instructions. Libraries were normalized, pooled, and sequenced on the Illumina® NextSEQ™ 500 with the NextSeq™ 500/550 Mid Output Kit v2.5 (150 Cycles) (#20024904), according to manufacturers' instructions. RNA-seq reads were aligned using Kallisto (Bray, N. L. et al., Nat Biotechnol (2016) 34: 525-527) to mouse genome version mm10, and expression levels were calculated using RSEM (Li, B. and Dewey, C.N. BMC Bioinformatics (2011) 12: 323). The data were normalized using TMM normalization, and differentially expressed genes were defined using the differential expression pipeline on the raw counts with a single call to the function Bioconductor package DESeq2 1.24.0 in R⁹¹ (FDR-adjusted P value <0.05). Heatmap figures were generated using pheatmap package (Kolde and Vilo, F1000 Research (2015) 4:574) and clustered using Euclidian distance. Functional enrichment analysis was performed using the Expander (Ulitsky, I., et al. Nat Protoc (2010) 5: 303-322) and g:Profiler (Raudvere, U. et al., Nucleic Acids Res (2019) 47: W191-W198) platforms.

### Human Gene Expression and Survival Analysis Data

Bulk gene expression of matched GBM patients and normal brain tissue was analyzed using the GEPIA portal (Tang, Z., et al., Nucleic Acids Res (2017) 45: W98-W102) based on The Cancer Genome Atlas (TCGA) and Genotype-Tissue Expression (GTEx) datasets. P<0.01 was considered statistically significant. Survival analysis was performed using the Chinese Glioma Genome Atlas (CGGA) (Zhao, Z., et al., Genomics Proteomics Bioinformatics (2021) 19: 1-12), P<0.05 was considered statistically significant. Single-cell analysis was performed on data of GBM patients (Isocitrate dehydrogenase 1 (IDH1)-negative, grade IV) published by Darmanis (Darmanis, S., et al., Cell Rep (2017) 21: 1399-1410) using Bbrowser (Le, T., et al. Biorxiv, (2020) 12.11.414136) (Ver 2.10.40; BioTuring Inc.). Cell linage clusters were defined based on the expression of known markers (TAMs, PTPRC, C1QC, and TMEM119; OPCs, PDGFRA, GPR17, OLIGO1, and AC058822.1; TAAs, SLC7A10, GJA1, and AQP4; Oligodendrocytes, MOBP, and MOG; endothelial cells, CD34 and PECAM1).

Statistical analysis Samples were randomly allocated into experimental groups at the start of each individual experiment. Genetically identical mice were randomly allocated to experimental groups at the start of the experiments. For in vitro experiments, biological samples were randomly allocated into experimental groups at the beginning of the experiment. Statistical analyses were performed with Prism 9.3 software (GraphPad), and the statistical tests used are indicated in the individual figure legends. P<0.05 was considered statistically significant. All error bars represent s.e.m. as noted in the individual figure legends. Displayed figures are representative. Box-and-whisker plots (Box plots) show median, interquartile interval, minimum, and maximum values.

### Data Availability

RNA sequencing data that support the findings of this study have been deposited in the Gene Expression Omnibus GSE193526, which can be accessed via www(dot)ncbi(dot)nlm(dot)nih(dot)gov/geo/query/acc(dot)cgi?acc=GSE193526 using Token ID ifsjyiagzfanjiz;

### Example 1

### Tumor-Associated Astrocytes (TAAs) Promote Glioblastoma Pathogenicity

Reactive astrocytes, characterized by elevated expression of glial fibrillary acidic protein (GFAP), have a considerable impact on the course of traumatic, ischemic, inflammatory, and degenerative diseases of the CNS. Astrocytes are one of the most abundant noncancerous cell types in glioblastoma (GBM), and reactive astrocytes are present around the tumor margins (FIG. 1A). Accumulating ex vivo data concerning the cross-talk between astrocytes and glioma cells, and specific inhibition of astrocytic signaling in medulloblastoma and brain metastasis tumor models, suggest that astrocytes might play a role in glioblastoma progression. However, the role reactive astrocytes play during glioblastoma pathogenicity in vivo is not well understood.

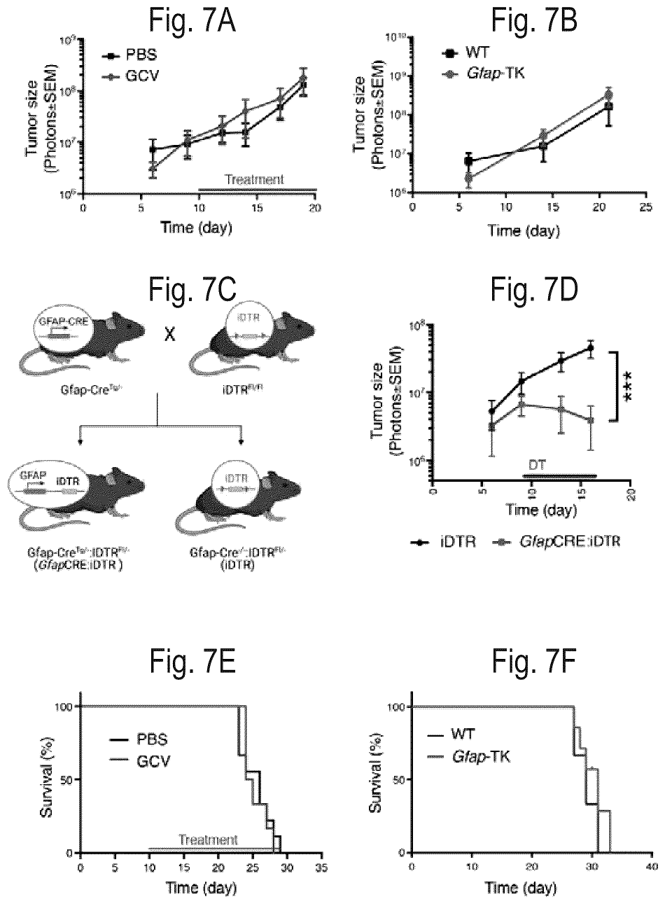

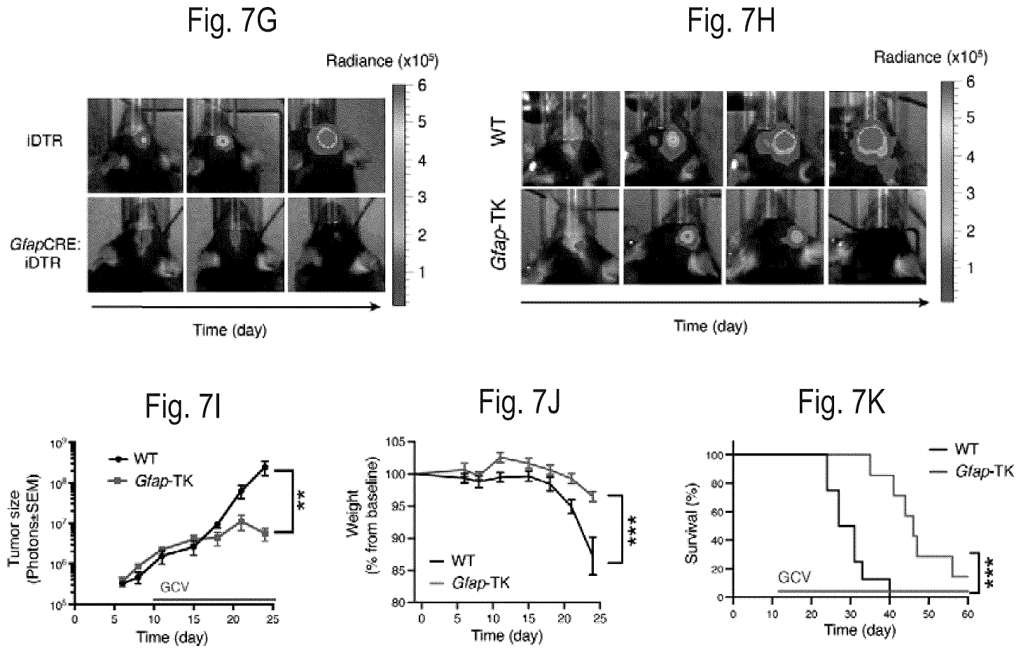

To study the role of TAAs in glioblastoma, the course of tumor progression was analyzed in mice expressing the herpes simplex virus thymidine kinase (HSVtk) under the Gfap promotor (Gfap-TK), in which Ganciclovir (GCV) administration can deplete reactive astrocytes. For this purpose, GFP⁺GL261-Luc (GL261) was intracranially injected into glioma cells, into syngeneic C57Bl/6 WT or into Gfap-TK littermates. Ten days after tumor cell implantation, once the tumors were established, the mice were treated with GCV for 7 days, before an examination of the presence of GFAP^{high} reactive astrocytes at the tumor margins (FIG. 1B). In line with previous reports of the specificity and efficiency of the Gfap-TK model (Mayo, L., et al., Nat Med (2014) 20: 1147-1156), it was uncovered that GCV administration to GBM-bearing mice, results in specific ablation of reactive astrocytes around the tumors in the Gfap-TK mice but not in WT mice (FIG. 1C). To test the functional contribution of reactive astrocytes to GBM progression, the same experimental paradigm was repeated (as depicted in FIG. 1B) and tumor growth was evaluated by bioluminescence imaging and histology (FIGS. 1D-G). While neither GCV treatment of WT mice nor genetic insertion of the HSVtk into Gfap-TK mice (without GCV treatment) affected GBM progression (FIGS. 7A-B), GCV treatment of GL261-bearing Gfap-TK mice did result in dramatic tumor regression (FIGS. 1D-G), suggesting that depletion of TAAs halts glioma growth.

Of note, clinical trials aimed at over-expressing HSVtk in high grade gliomas have shown minimal to no improvement in tumous burden and survival. Collectively, these data suggest that depletion of TAAs halts glioma growth.

To validate these findings an alternative astrocyte-depleting transgenic mice model was used, in which the diphtheria toxin (DT) receptor is expressed under the control of the murine Gfap promoter (GfapCRE:iDTR, FIG. 7C). Astrocytes are ablated in the GfapCRE:iDTR mice by administration of diphtheria toxinA (DT-A) (described and characterized in Schreiner, B., et al. Cell Rep (2015) 12: 1377-1384, which is fully incorporated herein by reference). Therefore, heterozygote GfapCRE mice were crossed with homozygotes mice harboring a Cre-inducible expression of DTR (iDTR). GfapCRE:iDTR and iDTR littermates were intracranially implanted with GL261 gliomas, and were treated with DT-A once the tumors were established. The results indicated a significant regression in tumor size in the GL261-bearing GfapCRE:iDTR mice, compared to their iDTR tumor-bearing littermates (FIGS. 7D and 7G), which is in agreement with the data obtained from the Gfap-TK astrocyte-depleting model (FIGS. 1D-G).

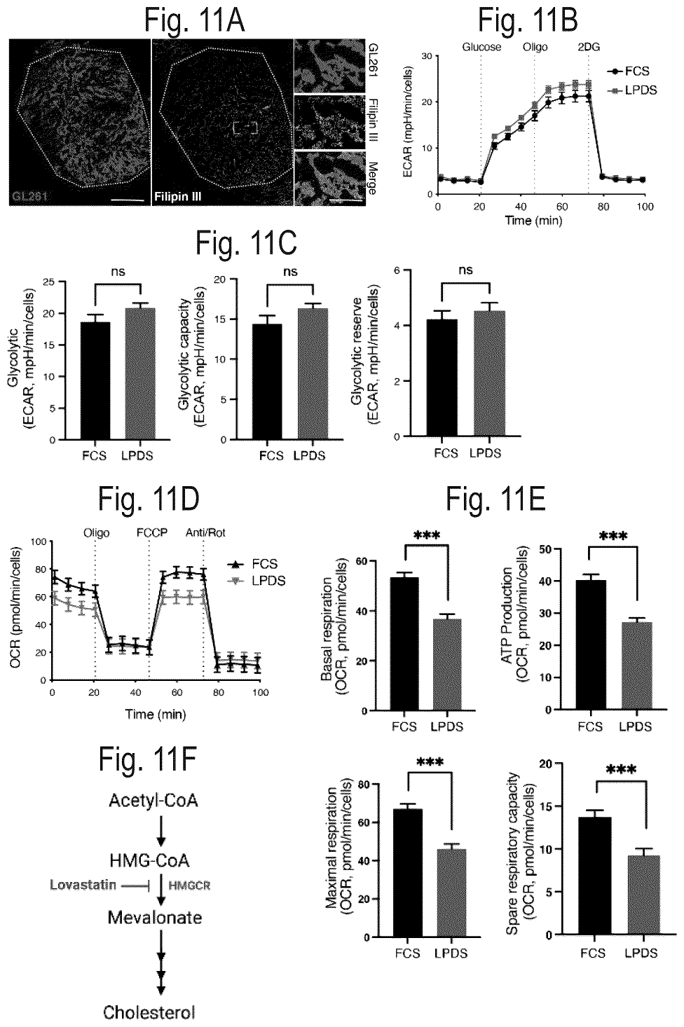

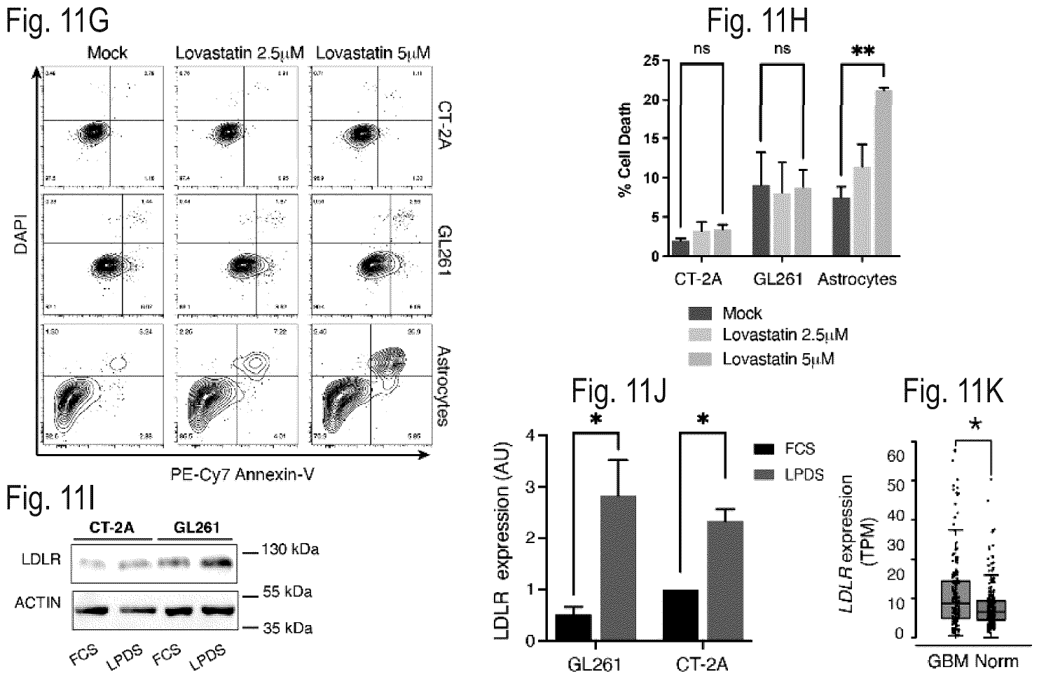

Next, the effect of astrocyte depletion on the disease pathophysiology was examined. To this end, GL261 cells were intracranially implanted into wild type (WT) or Gfap-TK littermates. The mice were treated with GCV (as depicted in FIGS. 1B and 1D) and the weight and survival were monitored. In accordance with the observed tumor regression, it was uncovered that astrocyte ablation significantly attenuates the weight loss in GL261-bearing Gfap-TK mice compared to their tumor-bearing WT littermates, and dramatically improves their survival (FIGS. 1H and 11, respectively). In contrast, GCV administration to GL261-bearing WT mice, or expression of the HSVtk transgene without GCV treatment, had no effect on survival (FIGS. 7E-F). Interestingly, the high survival rate of the GL261-implanted Gfap-TK mice persisted even after GCV administration was terminated following the death of all the mice in the WT group (FIG. 1I). It should be noted that prolonged treatment (3-4 weeks) of the GfapCRE:iDTR mice with DT-A led to a lethal bowel inflammation, presumably due to ablation of GFAP⁺ enteric glia in the distal small intestine. This was unrelated to tumor burden (data not shown), but as a result, the GfapCRE:iDTR model was only used to monitor tumor growth for a short regimen of DT-A treatment (7 days), well before the appearance of any DT-dependent clinical phenotype, and was not employed in the survival studies. Importantly, the observations that astrocytes support tumor pathogenicity were validated by the finding of similar results in an alternative syngeneic GBM model (CT-2A mouse glioma cells) (FIGS. 7H-K). Thus, these data suggest that reactive TAAs play a pivotal role in supporting glioma progression and tumor pathogenicity.

### Example 2

### TAAs Acquire a Pro-Tumorigenic Phenotype

To investigate the mechanisms by which reactive astrocytes support GBM pathogenicity, the present inventors has analyzed their transcriptional program using the RiboTag strategy, in which the expression of a haemagglutinin (HA)-tagged ribosome subunit, under the control of a CRE recombinase, allows for the analysis of cell-specific ribosome-associated mRNA. Mice carrying the floxed Rp122-HA allele (Sanz, E. et al. Cell-type-specific isolation of ribosome-associated mRNA from complex tissues. Proc National Acad Sci 106, 13939-44 (2009)) (Ribotag mice) were crossed with GfapCRE mice to generate GfapCRE:Rpl22HA mice. To implement the RiboTag method for the study of TAAs, GfapCRE:Rpl22HA mice were intracranially implanted with GL261 glioma cells (GBM) or injected with PBS (sham). Seventeen days later, RNA was retrieved from mouse brain extract (input) by anti-HA immunoprecipitation (IP-HA, FIG. 2A). First, the present inventor examined the colocalization of the HA tag specifically with astrocytes in the GfapCRE:Rpl22HA mice and the cell-type specificity of the obtained translatomes. Analysis of astrocyte-enriched RNAs (immunoprecipitated by an anti-HA antibody) confirmed enrichment of astrocyte-specific gene expression, and concomitant depletion of neuronal, oligodendroglial, and macrophage-specific gene expression (FIG. 2B). This was further validated by immunostaining (FIG. 2C). As the next step, the present inventors examined the transcriptional phenotype of the reactive astrocytes in the GBM microenvironment. Principal component analysis (PCA) demonstrated distinct differences between the tumor-associated astrocytes (GBM) and the astrocytes isolated from the sham control group (FIG. 8A). Indeed, the present inventor found that 3884 genes were differentially expressed (FDR adjusted P value <0.01, fold change >2) between the two groups of astrocytes (FIG. 2D). These included an increased expression of immune-associated genes (Chi3l1, Cd74), complement components (C1s, C3), chemokines (Ccl2), and proliferation (Mki67, Anxa2) transcription factors associated with glial support of brain tumors (Stat1, Stat3, Ahr) as well as genes associated with astrocyte crosstalk with microglial cells (Csf1, Cd44) or immunosuppression (e.g., Cd274, Gpnmb) (FIGS. 2E, 8B and 8E). In line with these findings, analysis of single-cell RNA-sequencing data (scRNAseq) released by Darmanis and colleagues of the TME of GBM patients (IDH1-negative, grade IV) identified a similar expression pattern in tumor-associated astrocytes (FIGS. 8C, and 9G). Functional analysis of the differentially expressed genes, identified enrichments in three main categories, namely perturbation of metabolic process, immune regulation, and cell proliferation (Figure and Table 1). Notably, the astrocyte immunosuppressive activity in the TME is not dependent on the CD8+ T-cell response, as the latter depletion did not modulate astrocyte-driven GBM pathogenicity (FIGS. 8F, 8G, 8H and 8I). Thus, these data identify a distinct phenotype of tumor-associated astrocytes in the glioblastoma microenvironment, whose transcriptomic characteristics resemble those of astrocytes from GBM patients. These results suggest that astrocytes might support glioma pathogenicity by directly promoting immunosuppression, regulating the neighboring immune cells, and contributing to the metabolic landscape of the tumor microenvironment.

TABLE 1
Functional enrichment analysis of tumor-associated astrocytes
(functional enrichment analysis of differently regulated transcripts
from tumor-associated astrocytes based on Gene Ontology (GO)).

| ID | Term ID | Term Name | P_{adj} |
|---|---|---|---|
| 1 | GO: 0007049 | Cell cycle | 2.810 × 10⁻¹⁷ |
| 2 | GO: 0008152 | Metabolic process | 7.455 × 10⁻¹⁸ |
| 3 | GO: 0022402 | Cell cycle process | 3.882 × 10⁻¹⁹ |
| 4 | GO: 0034097 | Response to cytokine | 5.456 × 10⁻¹⁹ |
| 5 | GO: 0044237 | Cellular metabolic process | 7.852 × 10⁻¹⁹ |
| 6 | GO: 0065009 | Regulation of molecular function | 1.892 × 10⁻¹⁷ |
| 7 | GO: 0071345 | Cellular response to cytokine | 6.642 × 10⁻¹⁸ |
| | | stimulus | |
| 8 | GO: 0071840 | Cellular component organization | 1.606 × 10⁻²⁶ |
| | | or biogenesis | |
| 9 | GO: 1903047 | Mitotic cell cycle process | 7.675 × 10⁻¹⁷ |
| 10 | GO: 0000278 | Mitotic cell cycle | 1.572 × 10⁻¹⁴ |
| 11 | GO: 0006950 | Response to stress | 1.257 × 10⁻¹⁵ |
| 12 | GO: 0010646 | Regulation of cell communication | 5.250 × 10⁻¹⁶ |
| 13 | GO: 0071704 | Organic substance metabolic | 9.935 × 10⁻¹⁵ |
| | | process | |
| 14 | GO: 0002376 | Immune system process | 1.398 × 10⁻¹¹ |
| 15 | GO: 0006807 | Nitrogen compound metabolic | 1.895 × 10⁻¹³ |
| | | process | |
| 16 | GO: 0006725 | Cellular aromatic compound | 1.421 × 10⁻¹² |
| | | metabolic process | |
| 17 | GO: 0006139 | Nucleobase-containing compound | 2.368 × 10⁻¹² |
| | | metabolic process | |
| 18 | GO: 0009893 | Positive regulation of metabolic | 9.465 × 10⁻¹⁴ |
| | | process | |
| 19 | GO: 0010604 | Positive regulation of | 4.478 × 10⁻¹² |
| | | macromolecule metabolic process | |
| 20 | GO: 0031325 | Positive regulation of cellular | 1.258 × 10⁻¹¹ |
| | | metabolic process | |
| 21 | GO: 0034641 | Cellular nitrogen compound | 1.218 × 10⁻¹¹ |
| | | metabolic process | |
| 22 | GO: 0044238 | Primary metabolic process | 4.418 × 10⁻¹² |
| 23 | GO: 0046483 | Heterocycle metabolic process | 4.138 × 10⁻¹³ |
| 24 | GO: 0051173 | Positive regulation of nitrogen | 9.325 × 10⁻¹² |
| | | compound metabolic process | |
| 25 | GO: 0001816 | Cytokine production | 2.656 × 10⁻¹⁰ |
| 26 | GO: 0002682 | Regulation of immune system | 6.102 × 10⁻¹¹ |
| | | process | |
| 27 | GO: 0006952 | Defense response | 1.819 × 10⁻⁸ |
| 28 | GO: 0006260 | DNA replication | 9.952 × 10⁻⁹ |
| 29 | GO: 0001819 | Positive regulation of cytokine | 7.640 × 10⁻⁸ |
| | | production | |
| 30 | GO: 0006261 | DNA-dependent DNA replication | 7.931 × 10⁻⁷ |
| 31 | GO: 0008283 | Cell population proliferation | 6.102 × 10⁻⁸ |
| 32 | GO: 0010547 | Positive regulation of cell | 4.337 × 10⁻⁹ |
| | | communication | |
| 33 | GO: 0031347 | Regulation of defense response | 2.701 × 10⁻¹⁰ |
| 34 | GO: 0031349 | Positive regulation of defense | 2.801 × 10⁻⁸ |
| | | response | |
| 35 | GO: 0035458 | Cellular response to interferon- | 1.396 × 10⁻⁷ |
| | | beta | |
| 36 | GO: 0044260 | Cellular macromolecule | 4.949 × 10⁻⁸ |
| | | metabolic process | |
| 37 | GO: 0044770 | Cell cycle phase transition | 2.302 × 10⁻⁹ |
| 38 | GO: 0044085 | Cellular component biogenesis | 2.027 × 10⁻¹⁰ |
| 39 | GO: 0043170 | Macromolecule metabolic process | 2.001 × 10⁻¹⁰ |
| 40 | GO: 0051301 | Cell division | 1.971 × 10⁻⁹ |
| 41 | GO: 0051726 | Regulation of cell cycle | 2.324 × 10⁻⁸ |
| 42 | GO: 0090304 | Nucleic acid metabolic process | 1.122 × 10⁻¹⁰ |
| 43 | GO: 1901987 | Regulation of cell cycle phase | 7.436 × 10⁻⁸ |
| | | transition | |
| 44 | GO: 1901990 | Regulation of mitotic cell cycle | 1.709 × 10⁻⁷ |
| | | phase transition | |
| 45 | GO: 0045935 | Positive regulation of nucleobase- | 2.624 × 10⁻⁷ |
| | | containing compound metabolic | |
| | | process | |
| 46 | GO: 0042127 | Regulation of cell population | 4.890 × 10⁻⁷ |
| | | proliferation | |
| 47 | GO: 0034645 | Cellular macromolecule | 3.575 × 10⁻⁶ |
| | | biosynthetic process | |
| 48 | GO: 0033993 | Response to lipid | 7.113 × 10⁻⁶ |
| 49 | GO: 0031323 | Regulation of cellular metabolic | 6.053 × 10⁻⁶ |
| | | process | |
| 50 | GO: 0019222 | Regulation of metabolic process | 1.206 × 10⁻⁶ |
| 51 | GO: 0008284 | Positive regulation of cell | 2.054 × 10⁻⁵ |
| | | population proliferation | |
| 52 | GO: 0002263 | Cell activated involved in | 5.501 × 10⁻⁵ |
| | | immune response | |
| 53 | GO: 0000280 | Nuclear division | 1.370 × 10⁻⁶ |
| 54 | GO: 0000082 | G1/S transition of mitotic cell | 1.451 × 10⁻⁴ |
| | | cycle | |
| 55 | GO: 0002697 | Regulation of immune effector | 1.190 × 10⁻⁴ |
| | | process | |
| 56 | GO: 0006955 | Immune response | 9.476 × 10⁻⁴ |
| 57 | GO: 0008608 | Attachment of spindle | 3.878 × 10⁻³ |
| | | microtubules to kinetochore | |
| 58 | GO: 0019221 | Cytokine-mediated signaling | 2.007 × 10⁻⁴ |
| | | pathway | |
| 59 | GO: 0030335 | Positive regulation of cell | 9.829 × 10⁻⁵ |
| | | migration | |
| 60 | GO: 0032270 | Positive regulation of cellular | 7.465 × 10⁻⁵ |
| | | protein metabolic process | |
| 61 | GO: 0050776 | Regulation of immune response | 1.550 × 10⁻⁵ |
| 62 | GO: 0060255 | Regulation of macromolecule | 1.416 × 10⁻⁶ |
| | | metabolic process | |
| 63 | GO: 0060759 | Regulation of response to | 6.124 × 10⁻⁵ |
| | | cytokine stimulus | |
| 64 | GO: 0080090 | Regulation of primary metabolic | 2.290 × 10⁻⁵ |
| | | process | |
| 65 | GO: 1902806 | Regulation of cell cycle G1/S | 3.190 × 10⁻³ |
| | | phase transition | |
| 66 | GO: 0140014 | Mitotic nuclear division | 1.703 × 10⁻⁵ |
| 67 | GO: 0060760 | Positive regulation of response to | 1.187 × 10⁻³ |
| | | cytokine stimulus | |
| 68 | GO: 0051247 | Positive regulation of protein | 3.679 × 10⁻⁴ |
| | | metabolic process | |
| 69 | GO: 0010556 | Regulation of macromolecule | 3.854 × 10⁻⁴ |
| | | biosynthetic process | |
| 70 | GO: 0006091 | Generation of precursor | 5.548 × 10⁻⁴ |
| | | metabolites and energy | |

### Example 3

### Astrocytes Modulate the Recruitment of GBM-Infiltrating Macrophages

The population of tumor-associated macrophages (TAMs) is made up of brain-resident microglia and bone marrow-derived myeloid cells from the periphery (monocyte-derived macrophages (MDM) from the periphery). Together they account for 30-50% of the tumor mass and their number is positively correlated with the tumor malignancy grade, and inversely correlated with overall survival in patients with recurrent glioblastoma (Hambardzumyan, D. et al. Nat Neurosci (2016) 19: 20-27).

Astrocytes regulate leukocyte infiltration to the CNS through several mechanisms that range from the regulation of blood-brain barrier permeability to the secretion of chemokines. Indeed the present inventor found that TAAs significantly upregulate their chemoattractant profile, compared to astrocytes isolated from the sham control (FIG. 3H), of which CCL2 and CXCL16, chemokines associated with TAMs tumor promoting activity were significantly expressed in astrocytes from GBM patients (FIG. 3I).

There are many factors that may mediate TAM chemoattraction to the GBM, but the main pathway for the recruitment of peripheral macrophages to the TAM compartment is considered to be via CCR2 (C—C motif chemokine receptor 2) signaling, which is triggered by CCL2 (C—C motif chemokine ligand 2) and CCL7 (C—C motif chemokine ligand 7). Analysis of the GBM Genome Atlas data revealed high levels of expression of CCR2, CCL2, and CCL7, which were associated with decreased survival in glioblastoma patients (FIGS. 9A-F). Under neuroinflammatory conditions, astrocytes regulate leukocyte infiltration to the CNS through several mechanisms, including the secretion of CCL2. However, the contribution of TAAs to the recruitment of TAMs during GBM progression is not well understood. To address this question, single-cell RNA-sequencing data from GBM patients were first screened for astrocytic expression of CCL2 and CCL7. The results indicated that while astrocytes express high levels of CCL2, CCL7 was barely detectable (FIG. 9G). Similarly, the levels of Ccl2 were elevated in astrocytes isolated from GL261-bearing mice using the RiboTagsystem (FIG. 2E) and in primary astrocytes treated with tumor-conditioned media generated using murine GL261 glioma (GBM-CM) (FIG. 3A). Interestingly, Ccl7 expression was not detected in Ribotag-isolated astrocytes, or GBM-CM treated primary astrocytes. In addition, the present inventor uncovered that astrocyte condition media (ACM), harvested from GBM-treated astrocytes (T-ACM), was significantly more efficient in inducing monocyte migration than control ACM (FIG. 3B). Importantly, the use of anti-CCL2 neutralizing antibodies to block CCL2 signaling, inhibited this increase in monocyte migration, implicating CCL2 as the main chemoattractant in GBM-induced astrocyte recruitment of the monocytes (FIG. 3C). To investigate whether these observations indicate that TAAs regulate TAM recruitment to the TME, the recruitment of TAMs into the TME of GL261-bearing WT and Gfap-TK mice treated with GCV (as in FIG. 1B) was monitored. It was uncovered that depletion of reactive astrocytes with GCV significantly reduces the accumulation of IBA-1⁺ TAMs in the TME (FIGS. 3D-E). Similar results were obtained by FACS analysis of the frequency of TAMs (CD11b⁺CD45⁺) cells in the TME (FIGS. 3F-G), suggesting that reactive astrocytes directly control the recruitment of TAMs to the GBM TME and that this process is predominantly mediated by CCL2.

### Example 4

### TAAs Direct TAMs Towards an Immunosuppressive Phenotype

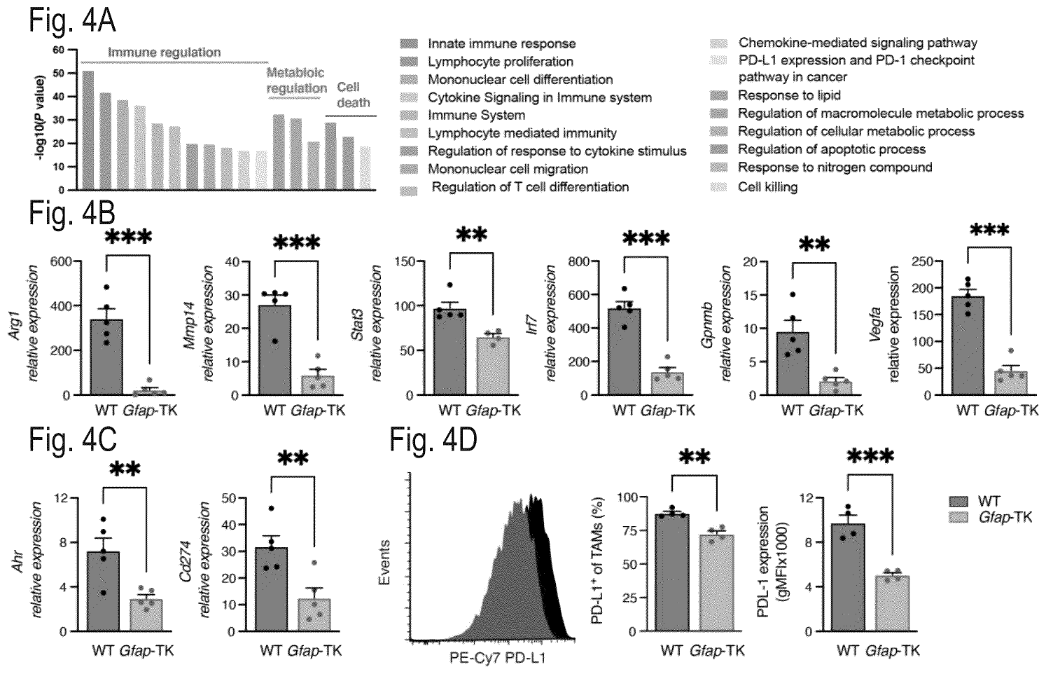

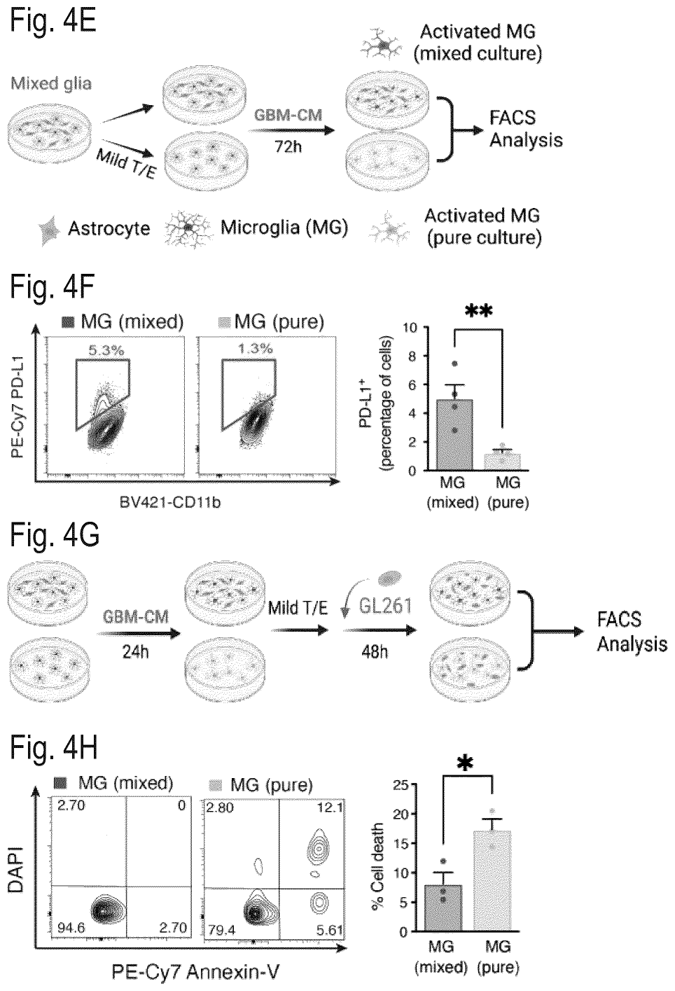

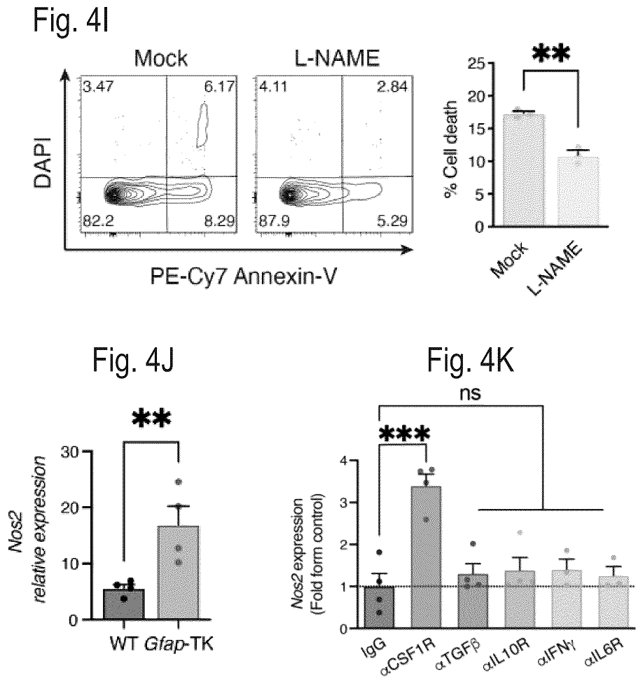

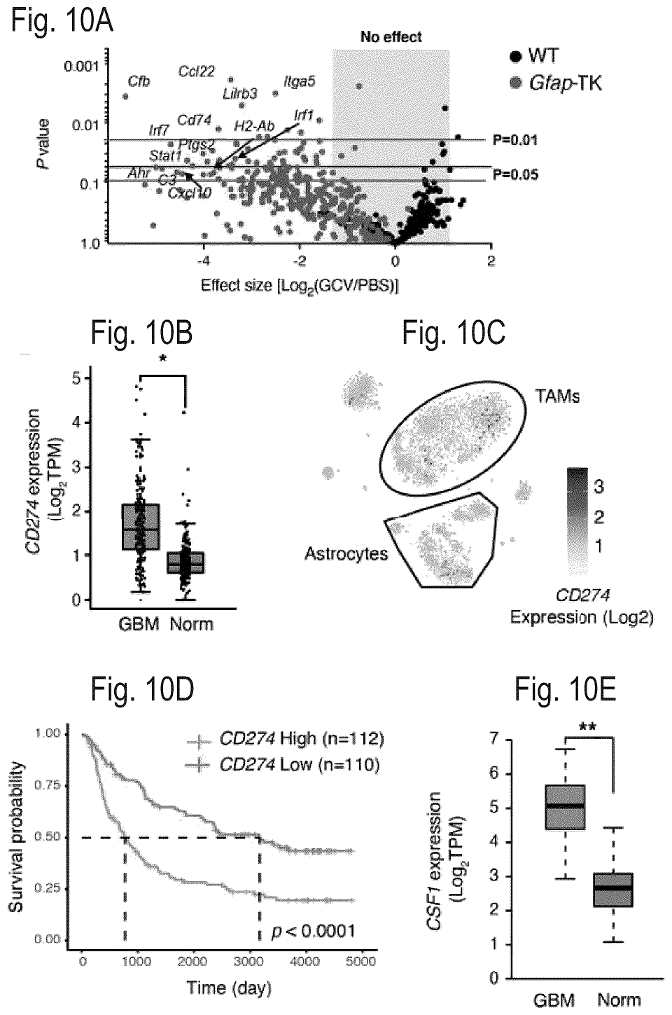

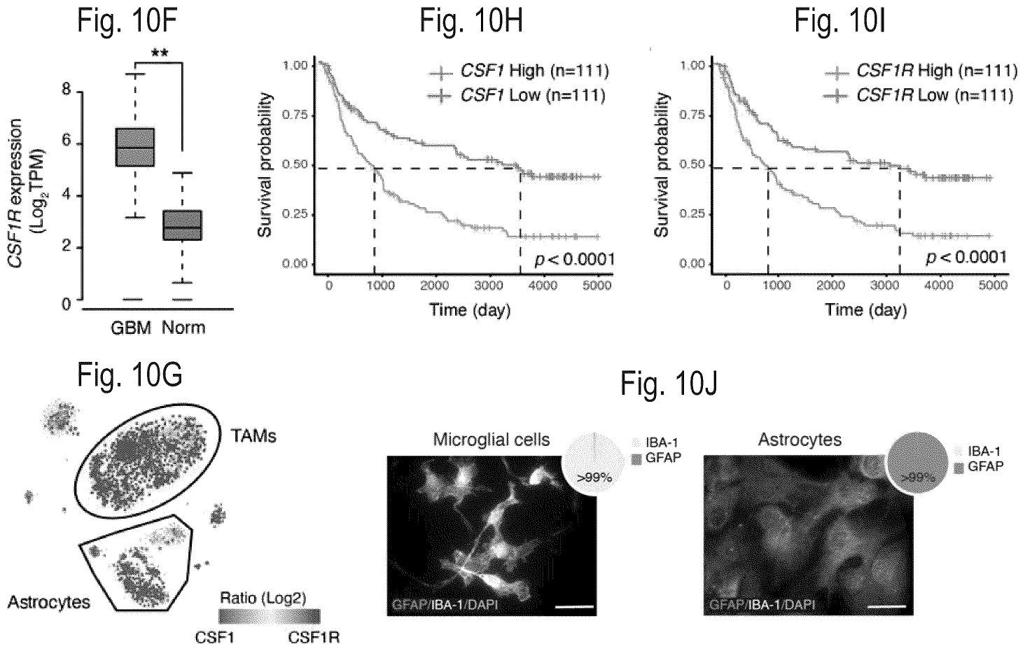

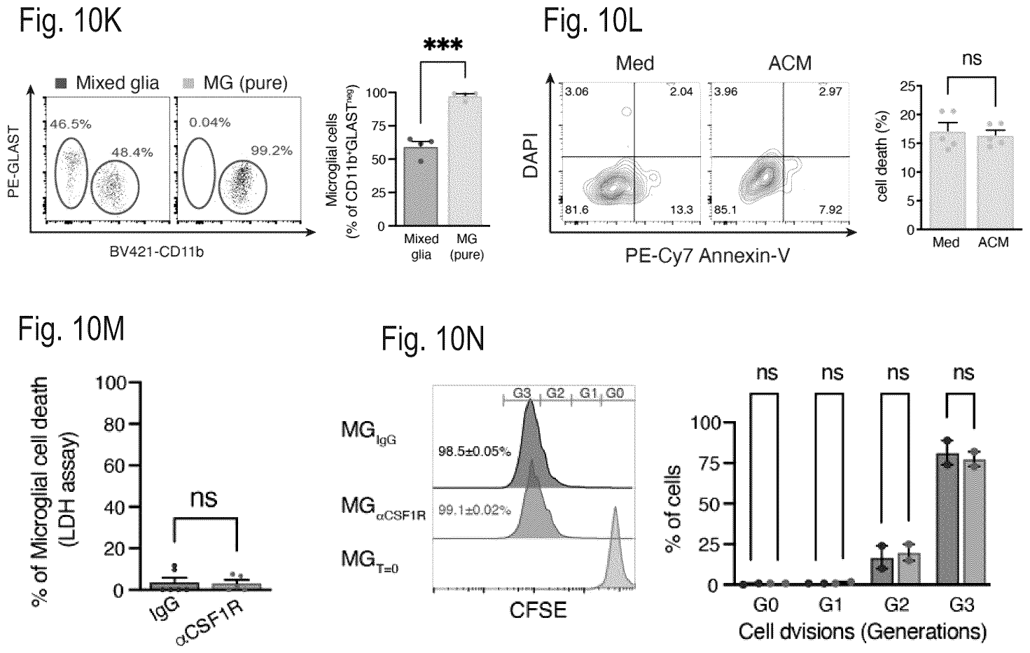

TAMs support glioblastoma pathogenicity, by promoting tumor growth, immunosuppression, and resistance to therapy. They acquire this distinct pro-tumor phenotype as a result of direct interactions with the tumor and the TME, although the mechanisms concerned are not entirely understood. It was previously demonstrated that astrocytes regulate the immunological profile of resident microglial cells and CNS-infiltrating monocytes during neuroinflammation (Mayo, L., et al., Nat Med (2014) 20: 1147-1156; Mayo, L., et al., Brain (2016) 139: 1939-1957) and Heiland and colleagues reported a similar cross-talk between astrocyte and microglial cells in in-vitro models of glioma (Henrik Heiland, D., et al., Nat Commun (2019) 10: 2541). To investigate whether reactive astrocytes also control TAMs activity in the context of tumor pathology, the transcriptome from TAMs harvested from GL261-bearing WT and from Gfap-TK mice that were treated with GCV were analyzed to deplete their reactive astrocytes (FIGS. 4A-C and FIG. 10A). It was uncovered that astrocyte depletion was accompanied by significant differences in the mRNA expression profile of TAMs. These changes were identified as factors associated with the functional enrichment of immune regulation pathways, TAM metabolome, and regulation of cell death (FIG. 4A and FIG. 10A). Notably, astrocyte depletion reduced the expression of a number of the hallmark genes associated with the tumor-promoting TAM phenotype, including Arg1, Mmp14, Stat3, Irf7, Gpnmb, and Vegfa, and aryl hydrocarbon receptor (Ahr), which regulates TAMs activity in GBM (FIG. 4B). Promoting immunosuppression is one of the mechanisms by which TAMs are known to contribute to GBM progression and resistance to emerging immunotherapies. Programmed death-ligand 1 (PD-L1), encoded by CD274, is among the prominent members of the checkpoint inhibitor family and has shown promising therapeutic potential in a variety of different neuropathologies, including GBM. Analysis of the scRNAseq data derived from the TME of GBM patients revealed that the high expression of CD274 seen by The Cancer Genome Atlas (TCGA) analysis in GBM patients can be attributed predominantly to TAMs and astrocytes. Importantly, a reduction in the expression of CD274 is associated with improved survival (FIGS. 10B-D). It was uncovered that depletion of reactive astrocytes significantly attenuated Cd274 induction and PD-L1 expression by TAMs (FIGS. 4C-D), suggesting that TAAs program the TAMs to support GBM pathogenicity and induce an immunosuppressive environment. To test this hypothesis, the effects of the presence or absence of astrocytes on microglial expression of PD-L1 in response to GBM-CM stimulation was studied (FIG. 4E). In line with the in-vivo analysis (FIG. 4D), the expression of PD-L1 was inhibited in the absence of astrocytes (FIG. 4F). These data therefore support the notion that astrocytes can directly reprogram the microglial cells to promote the immunosuppressive profile of the TME.

Most microglial cells in non-malignant or regressing tumors have a pro-inflammatory activity that may promote tumor lysis. Accordingly, pro-inflammatory (M1-like) microglial cells, which express the inducible nitric oxide synthase (iNOS), have been shown to induce glioma cell death (Xue, N., et al., Sci Rep (2017) 7: 39011). The transcriptomic analysis suggested that astrocyte depletion regulates the TAMs cytotoxic potential and can modulate nitric oxide (NO) metabolism in the TME (FIGS. 2F and 4A, and Table 1, above). As the first step to confirm this possibility, the ability of astrocytes to regulate the microglial-mediated glioma cytotoxicity was examined (FIGS. 4G-H). For this purpose, microglial cells were pre-activated with GBM-CM in the presence or absence of astrocytes for 24 hours, to allow for astrocyte modulation of microglial activity. The astrocytes were then removed by mild trypsin/EDTA (T/E) treatment, and the microglial cells were co-incubated with GPF⁺-GL261 cells for 48 hours (as illustrated in FIG. 4G). Glioma cells were then isolated based on their GFP expression, and their viability was determined (FIG. 4G). It was uncovered that astrocytes attenuate the microglial-dependent killing of glioma cells by 48±12.6% (FIG. 4H).

Next the present inventor has investigated whether this immunosuppressive function on microglial cell cytotoxicity towards glioma cells is a specific immunosuppression response or a general response to the withdrawal of tropic support by astrocytes. Microglial cells were treated with ACM (astrocyte condition media), which was generated from astrocyte cultures without previous contact with neither glioblastoma not microglial cells, or control medium (Med), and co-incubated with GFP⁺-GL261 cells as in FIG. 4H. It was found that ACM did not significantly regulate the microglial-dependent killing of glioma cells (p=0.678 by two sided Student's t-test, FIG. 10L), suggesting that astrocytes play a direct and specific immunosuppressive function in regulating the microglial response to glioma cells.

To continue and explore this hypothesis, the experiment was then repeated in the presence of the NO synthase inhibitor, L-NAME (N-Nitro-L-Arginine Methyl Ester), which has been shown to inhibit iNOS-depend neuroinflammation and microglial-induced cell death (Mayo, L. & Stein, R., Cell Death Differ (2007) 14: 183-186). It was observed that inhibition of NO synthesis also attenuates microglial-induced killing in the present system, suggesting that it is at least partially dependent on NO production (FIG. 4I). Accordingly, a significant upregulation in Nos2 (the transcript encoding iNOS) expression in microglial cells isolated from GCV-treated GL261-bearing Gfap-TK, compared to WT mice, was also detected (FIG. 4J). Next, the signaling cascade by which astrocytes govern microglial reprogramming was studied. Treating mixed glia cultures (containing microglial cells and astrocytes) with neutralizing antibodies to CSF1, TGFβ, IL-10, IFNγ, or IL-6 signaling, and analyzing GBM-CM stimulated microglial induction of Nos2, indicated that blockade of CSF-1R induced its expression. These results suggest that astrocyte-driven CSF1 inhibits microglial Nos2 induction (FIG. 4K). Of note, blocking colony stimulating factor 1 receptor (CSF-1R) signalling did not significantly regulate microglial viability or microglial proliferation (FIGS. 10M and ION). Indeed, analysis of TCGA data, and scRNAseq data of the GBM TME, identify the upregulation of CSF1 and CSF-1R expression (mainly in astrocytes and TAMs, respectively), which is correlated with lower patient survival (Figures l0E-I). Taken together, the data suggest that astrocytes re-programme the microglial cells to promote immunosuppression and support tumor survival, a process that is partly mediated by CSF1 signaling and iNOS expression.

### Example 5

### Astrocyte-Derived Cholesterol Supports Glioma Survival

The metabolism of cancer cells adapts during transformation, and the altered cellular metabolism that is a hallmark of gliomas, may be a promising source of druggable targets for therapy. This is particularly pertinent for glioblastoma progression, as the CNS is isolated from the circulation by the BBB and depends heavily on astrocytes for metabolic homeostasis. Analysis of the TAA transcriptome suggests a significant perturbation in the metabolic network (FIG. 2F). Genome-scale metabolic modeling (described in Opdam, S., et al., Cell Syst (2017) 4: 318-329 e316) was used to identify which of the astrocyte core metabolic pathways are regulated by the tumor, since this method was previously shown to model human brain metabolism in health and disease (Richelle, A., et al., Cell Rep Methods (2021) 1). For this purpose, the transcriptome data was analyzed using the Integrative Metabolic Analysis Tool (iMAT) to predict the metabolic flux activity (Table 2, below). Interestingly, the results revealed a significant enrichment of the cholesterol metabolic pathway (p=1.22×10⁻¹⁵).

TABLE 2
Metabolic pathway enrichment in glioblastoma-associated astrocytes

| Metabolic pathway | P-value |
|---|---|
| Bile Acid Biosynthesis | <10⁻¹⁶ |
| Hyaluronan Metabolism | <10⁻¹⁶ |
| Stilbene, coumarine and lignin biosynthesis | <10⁻¹⁶ |
| Cholesterol Metabolism | 1.22 × 10⁻¹⁵ |
| Folate Metabolism | 4.04 × 10⁻⁷ |
| Ubiquinone Biosynthesis | 3.88 × 10⁻⁶ |
| Carnitine shuttle | 1.83 × 10⁻⁵ |
| Eicosanoid Metabolism | 7.21 × 10⁻⁵ |
| Transport, Lysosomal | 1.13 × 10⁻⁴ |
| Transport, Peroxisomal | 3.72 × 10⁻⁴ |
| Transport, Endoplasmic Reticular | 3.91 × 10⁻⁴ |
| Pentose and Glucuronate Interconversions | 3.85 × 10⁻³ |
| Nucleotide Sugar Metabolism | 6.69 × 10⁻³ |
| Glycosylphosphatidylinositol (GPI)-anchor biosynthesis | 6.82 × 10⁻³ |

Cholesterol is predominantly localized to bilayer membranes such as the cell membrane and the mitochondrial membrane. It is essential for cellular biological functions ranging from signal transduction to modulation of critical mitochondrial-governed processes such as oxidative phosphorylation and regulation of apoptosis. Many cancers display a high cholesterol content, which is thought to support tumor growth and the viability and activity of cells in the TME, and targeted disruption of cholesterol metabolism was shown to be beneficial in adult and childhood brain tumors (Phillips, R. E., et al., Proc Natl Acad Sci USA (2019) 116: 7957-7962; Villa, G. R., et al., Cancer Cell (2016) 30: 683-693). Analysis of cholesterol distribution in the brain of GL261-bearing mice, identified higher cholesterol levels in the tumor cells, than in the surrounding brain tissue, which agrees with previous reports (Villa, G. R., et al., Cancer Cell (2016) 30: 683-693) that glioma cells take up significantly more extracellular cholesterol than non-neoplastic cells (FIG. 11A). Importantly, cholesterol synthesis in the brain is mainly dependent on astrocytes, as the BBB effectively prevents the uptake of lipoprotein-bound cholesterol from the circulation. Indeed, perturbations in astrocyte-derived cholesterol have been associated with a variety of neuropathologies. However, the role of astrocyte-derived cholesterol in GBM progression is unknown.

Since cholesterol has been found to be specifically enriched in the mitochondrial membrane of cancer cells where it decreases membrane fluidity, and reduces the mitochondria sensitivity to stress, it was hypothesized that cholesterol produced by the TAAs could be used to regulate GBM pathogenicity. To test this hypothesis, the dependency of glioma cells on exogenous cholesterol was first studied by removing the cholesterol-carrying lipoproteins from the culture media and assessing the mitochondrial bioenergetic potential as an indication of cellular stress. Interestingly, although like many other cancer cells, glioma cells can utilize glycolysis for growth (Warburg effect), they rely upon mitochondrial oxidative phosphorylation (OXPHOS) to support aggressive tumor growth. Therefore, the extracellular acidification rate (ECAR), indicative of aerobic glycolysis, and oxygen consumption rate (OCR), as a readout of OXPHOS, were monitored in GL261 and CT-2A glioma cells cultured for 18 hours in media supplemented with lipoprotein-deficient serum (LPDS) or normal serum (FCS) (FIGS. 5A-D, and FIGS. 11B-E). It was uncovered that lipoprotein depletion had little to no effect on ECAR, and the addition of glucose did not affect the cytosolic-based glycolysis rate or glycolytic capacity in the glioma cell lines, with only minor effect on the GL261 glycolytic reserve (FIGS. 5A-B, and FIGS. 11B-C). However, in absence of lipoproteins, there was significant inhibition of the OCR potential as well as a decrease in basal respiration and ATP production by the glioma cells. Notably, OCR was attenuated when oxygen consumption was uncoupled from ATP production by FCCP (carbonyl cyanide4-(trifluoromethoxy)phenylhydrazone), demonstrating a significant reduction in the glioma cells' maximal respiration and spare respiratory capacity. Thus, suggesting that removal of exogenous cholesterol resulted in mitochondrial stress, specifically targeting the OCR activity of the glioma cells, which is required for the tumor progression.

Mitochondrial stress is often associated with cell death, as is the reduction in cellular cholesterol levels. Therefore, the viability of the murine glioma cells and primary astrocytes subjected to prolonged deprivation of cholesterol were analyzed. It was reasoned that while astrocytes, which can de-novo synthesize cholesterol, will resist exogenous cholesterol deprivation, the murine glioma cells will be highly vulnerable. To address this objective, primary astrocytes and glioma cells were treated with lovastatin, which inhibits the 3-hydroxy-3-methylglutaryl-CoA reductase (HMGCR), a rate-limiting enzyme in the cholesterol synthesis pathway. It was uncovered that suppressing endogenous cholesterol synthesis did not affect glioma viability, but led to significant cell death of primary astrocytes (FIGS. 11F-H). Conversely, depriving the murine glioma cells of lipoprotein-bound cholesterol, resulted in substantial cell death, while astrocyte viability remained unaffected (FIG. 5E), suggesting that the glioma cells are indeed dependent on exogenous cholesterol. Consistent with this interpretation, removing the cholesterol-carrying lipoproteins from the culture media, increased the expression of the low-density lipoprotein receptor (LDLR), which mediates cholesterol uptake by the cells, a finding that was also noted in GBM patients (FIGS. 11I-K). Furthermore, exogenous cholesterol overcame the deleterious effects of lipoprotein-bound cholesterol deprivation on glioma viability (FIG. 5F-G). Similarly, activating the liver X receptor (LXR) signaling pathway with the endogenous ligand 24(S)-hydroxycholesterol (24-OHC), which results in a 24-OHC dose-dependent LDLR degradation, thus limiting cholesterol uptake by the cells, led to dramatic cell death in both GL261 and CT-2A glioma cell lines (FIGS. 5H-K). Collectively, these data demonstrate that glioma cells depend on exogenous cholesterol for their survival. Importantly, these findings align with similar data described recently by Villa and colleagues (Villa, G. R., et al., Cancer Cell (2016) 30, 683-693) that demonstrate a dependency of human gliomas on LXR-depended cholesterol uptake.

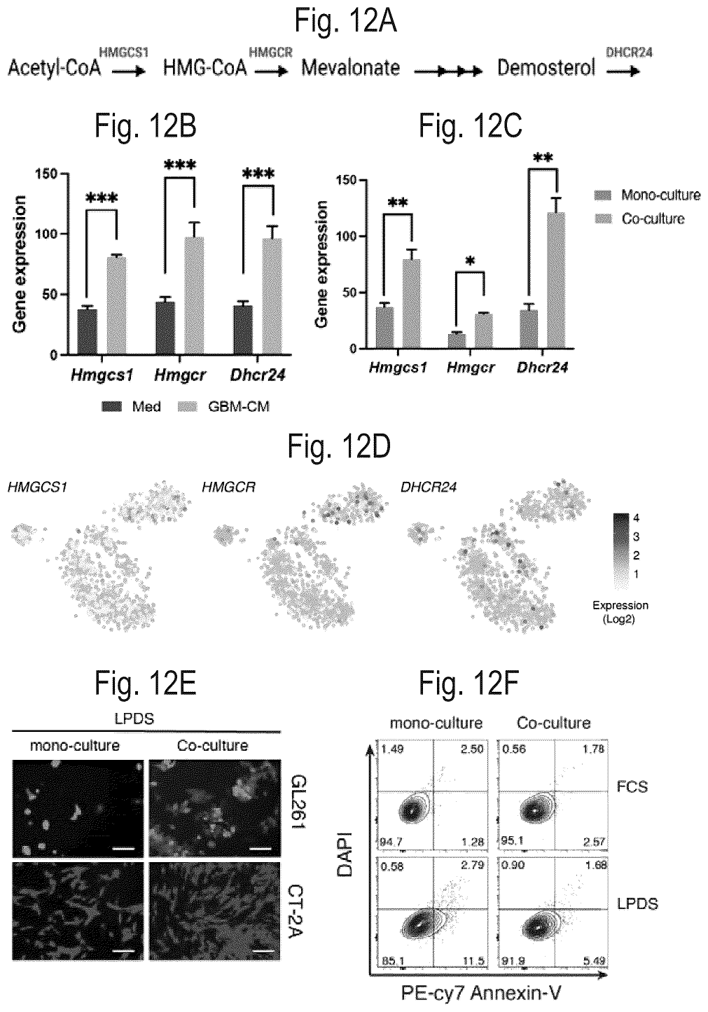

Next, the dynamics of cholesterol metabolism in the astrocyte-glioma cross-talk were investigated. Treating astrocytes with GBM-CM, or co-culturing them with glioma cells, induces the expression of rate-limiting enzymes in the cholesterol de-novo synthesis pathway, including Hmgcs1, Hmgcr, and Dhcr24 (FIGS. 12A-C). Again, this increase is in line with the elevated expression levels of these genes seen in scRNAseq data of astrocytes from GBM patients (FIG. 12D), suggesting that glioma cells may elicit astrocytes to support their metabolic requirement for cholesterol. Culturing glioma cells in the presence (co-culture) or absence (mono-culture) of astrocytes, and subjecting them to lipoprotein deprivation, revealed a dramatic rescue of the murine GL261 and CT-2A glioma cells by the astrocytes (FIGS. 5L-M and FIG. 12E). Moreover, a similar recovery from cholesterol starvation was found in human U87EGFRvIII glioma cells in the presence of human primary astrocytes (FIG. 5N and FIG. 12F). Taken together, these data suggest that the glioma cells rely on the uptake of exogenous cholesterol for survival, and that human and murine astrocytes can meet these metabolic requirements for cholesterol and support glioma cell survival.

### Example 6

### Astrocytic ABCA1-Driven Cholesterol Efflux Promotes Tumor Progression

Intracellular cholesterol trafficking in the brain is mediated mainly by the sterol transporters ABCA1 (ATP-binding cassette transporter A1) and ABCG1 (ATP binding cassette subfamily G member 1). The current analysis of TCGA data identified a high expression of both proteins in the CNS of GBM patients (FIG. 6A and FIG. 13A), although the analysis of scRNAseq of the GBM microenvironment identified that TAAs predominantly express ABCA1 (FIG. 6B). Transcriptomic analysis of the TAAs isolated from GBM-bearing mice transcriptome, similarly detected high levels of Abca1 induction, with an absence of Abcg1 transcripts (FIG. 6C). Concomitantly, expression of ABCA1 was detected by immunofluorescence in reactive astrocytes surrounding the GBM tumor in mice (FIG. 6D). These also showed that exposure to GBM-CM directly induces ABCA1 expression in astrocytes (FIG. 6E), thus suggesting that ABCA1 plays a role in the astrocyte-mediated cholesterol shuttling to the tumor.

It was therefore reasoned by the present inventors that knocking down astrocytic ABCA1 expression, would limit the astrocyte-derived cholesterol available to the tumor cells and enable to evaluate the extent to which it contributes to tumor pathology. To investigate this hypothesis, the present inventor has generated ABCA1-targeting lentiviruses using RNA interference (RNAi) or the CRISPR-Cas9 systems (FIG. 6F and FIGS. 13C, respectively). The present inventor has transduced primary astrocytes with the lentiviruses targeting ABCA1 [shAbca1, sgAbca1(#1; SEQ ID NO: 5) and sgAbca1(#2; SEQ ID NO: 6)] or appropriate controls [non-targeting shRNA (shNT; SEQ ID NO: 1) or sgRNA targeting the luciferase gene (sgLuc2; SEQ ID NO: 4), respectively]. The present inventor found that all Abca1-targeting sequences significantly knocked-down ABCA1 expression compared to their respective controls (FIGS. 13D-G). The present inventor next studied whether astrocytic ABCA1 knock-down would attenuate astrocyte-mediated rescue of glioblastoma cells from cholesterol depredation. The transduced primary astrocytes were co-cultured with glioblastoma cells in a cholesterol-free medium, and their survival was analyzed (as in FIG. 5L). The present inventor found that all Abca1-targeting sequences significantly inhibited the astrocyte-mediated rescue of the glioma cells (FIGS. 13H-J). Thus, these data demonstrate that ABCA1 plays an important role in the astrocyte-mediated cholesterol shuttling to the glioma cells.

To further investigate the initial hypothesis and determine the role of astrocytic ABCA1 expression on tumour pathogenicity, the validated non-targeting (NT) or Abca1-specific shRNAs were delivered to reactive astrocytes in the GBM TME, by using a lentivirus-based system optimized for astrocyte-specific knock-down in vivo (Mayo, L., et al., Nat Med (2014) 20: 1147-1156) (FIG. 6F). In this system, the truncated GFAP promoter, GfaABC1D (SEQ ID NO: 3), drives the expression of a miR30-based shRNA and a GFP reporter. Intracranial injection of shRNA-encoding lentivirus to GL261-bearing mice indeed confirmed that the GFP reporter was detected exclusively in the GFAP⁺ astrocytes (FIG. 13B), and significantly reduced astrocytic Abca1 expression (FIG. 6G).

As the next step, it was investigated whether knockdown of Abca1 in astrocytes affects the cholesterol content of tumor cells. To address this objective, GL261-bearing mice were intracranially injected the Gfap-shNT or Gfap-shAbca1 and cholesterol accumulation in the tumor cells was assessed using Filipin III staining. In accordance with the hypothesis, knockdown of astrocytic Abca1 led to a significant decrease of glioma cholesterol (FIGS. 6H-I) and concomitant induction of apoptotic cell death (FIGS. 6J-K). Moreover, inhibiting the efflux of cholesterol from the TAAs significantly regressed tumor growth and prolonged survival of the GL261-bearing mice (FIGS. 6L-N). Notably, analysis of TCGA data indicated that reduced expression of ABCA1 was associated with increased survival in patients with GBM (FIG. 6O). Collectively, these data suggest that glioblastoma pathogenicity is dependent upon astrocyte-derived cholesterol.

### Analysis and Discussion

Whereas it is becoming increasingly clear that astrocytes play an important role in a number of neurological disorders, little is known about the nature of astrocyte contribution to glioblastoma pathogenicity in vivo. Here, the present inventor first addressed this question by genetically depleting GFAP^{high} reactive astrocytes in adult immunocompetent mice, a method that has proven valuable for understanding the role of astrocytes in various neuropathologies. Two models were used: the GfapCre:iDTR mice model, which depletes GFAP^{high} astrocytes, and the Gfap-TK mice model, which also requires the astrocytes to be in a proliferative state [a characteristic feature of both human and murine TAAs (FIGS. 2E and 2F)]. The results indicate that ablation of reactive glioma-associated astrocytes from the TME halts tumour progression, regresses established gliomas and markedly enhances survival of the animals in both GBM models (FIGS. 1A-I and FIGS. 7A-K), suggesting that TAAs play a pivotal role in controlling glioblastoma pathogenicity.

To unravel the molecular circuits by which astrocytes govern GBM pathogenicity, the present inventor performed RiboTag-based RNA-seq on TAAs. RiboTag restricts analysis to ribosome-associated mRNAs that are likely to be in active translation, thereby reflecting the cellular protein expression profile, while minimizing any bias resulting from cell isolation-based methods. The results identify unique transcriptomic reprogramming of glioma-associated astrocytes, with the capacity to directly induce immunosuppression and control the immunological compartment and the metabolic landscape of the TME (FIGS. 2A-F and FIGS. 8A-B and 8E-I). Importantly, the astrocyte gene expression dataset is validated by broad agreement with other RNA-seq profiles of astrocytes from the GBM patients including the scRNAseq analysis of the GBM (IDH1-negative, grade IV) TME by Darmanis et al. (Cell Rep. 2017, 21: 1399-1410) and the analysis of GBM-associated astrocytes isolated by immunopanning by Henrik Heiland et al. (Nat. Commun. 2019, 10: 2541).

TAMs, which possess both tumour-promoting and immunosuppressive capacities, are abundant in the TME. Since their accumulation has been shown to be reversely correlated with patient survival, and given the abysmal prognosis of GBM patients, there is growing interest in developing novel therapeutics to target TAM activity. However, whether these activities are predominantly shaped by the non-neoplastic cells in the TME milieu or by the malignancy itself is unknown. Here the present inventor demonstrates that astrocytes have an important role in reprogramming TAMs in the glioma microenvironment. The present inventor found that astrocytes regulate the recruitment of TAMs to the tumour (FIGS. 3A-I) and control various aspects of the TAM tumour-promoting and immunosuppressive phenotype (FIGS. 4A-K and FIGS. 10A-N). In addition, the CCL2-CCR2 and CSF1-CSF1R axes were implicated in the molecular mechanisms by which the astrocytes control the TAM compartment and thus shape tumour-specific immunity (FIGS. 3A-G and FIGS. 4A-K). The present inventor has also detected an increased expression of these genes in GBM patient-derived scRNAseq and in TCGA data, which was inversely correlated to patient survival (FIGS. 9A-G and FIGS. 10A-N). Similar expression and survival patterns were also found for CD274, the transcript encoding PD-L1 (10A-N). These findings are in agreement with previous reports showing the perturbations of CCL2, CSF1 or PD-L1 signalling are able to regulate the TAMs niche in the TME and affect tumour pathogenicity in mouse models of GBM. Regulating TAMs activity, for example, by using minocycline that blocks microglial activation or the specific CSF-1R inhibitor PLX3397, reduces glioma expansion in experimental glioma mouse models. Unfortunately, so far, the clinical application of these basic science advances has been disappointing, and strategies designed to silence TAM function have not translated well to human clinical trials. Immunotherapies have demonstrated limited efficacy in patients with glioblastoma. However, in recent early phase clinical trials, the use of nivolumab or pembrolizumab, which targets the PD-L1/PD-1 axis, has shown promising outcomes. Thus, suggesting that to overcome the tumour-supporting properties of the TAMs, it might not be enough to target specific aspects of TAM biology. Instead, there is a need to develop strategies to harness the astrocytes to reprogramme the TAMs and redirect them back to fight cancer.

Alterations in cellular metabolism, governed by the interaction between tumour genotype and its microenvironment, are a hallmark of many malignancies including brain gliomas. Cancer cells, due to their genetic abnormalities, aggressive proliferation rate and metabolic restrictions of their microenvironment, may become dependent on factors that are not themselves oncogenic. This process is known as non-oncogene addiction or non-oncogene codependency and opens up treatment possibilities. Accordingly, two of the main genetic abnormalities found in GBM tumours are the amplification of the gene encoding for epidermal growth factor receptor (EGFR), with nearly half of the cases bearing the gain-of-function EGFR variant III (EGFRvIII) alteration; And, the recurrence of mutations in the genes Isocitrate dehydrogenase 1 (IDH1) and Isocitrate dehydrogenase 2 (IDH2), which are components involved in the tricarboxylic acid cycle. Both EGFR and IDH have been associated with the regulation of glioma bioenergetics, which favours OXPHOS for aggressive growth, over glycolysis. Accordingly, treatment of patient-derived GBM xenograft mice with Gboxin, an OXPHOS inhibitor, suppresses the growth of tumours with EGFR or IDH1 mutations. In recent years, several important findings as to the role of IDH mutations in glioblastoma pathogenicity, have linked tumour metabolism and immune perturbations of T cells and TAMs in TME. Accordingly, Bunse et al. (Nat. Med. 2018, 24: 1192-1203) have elegantly demonstrated that the oncometabolite (R)-2-hydroxyglutarate, produced by IDH mutations, suppresses T-cell activity, while Klemm et al. (Cell, 2020, 181: 111-121) found that IDH mutation status shapes TAM composition (favouring microglial recruitment over monocyte-derived macrophages) and phenotype (e.g. suppressing GPNMB induction) in TME, suggesting that understanding the immunometabolic cross-talk between the glioma cells and the nonneoplastic cells in the TME is becoming increasingly important for finding more effective ways to treat glioblastoma patients: specifically, by targeting the tumour altered cellular metabolism, and the unique metabolic vulnerabilities enforced by the blood-brain barrier. However, currently, the metabolic landscape of the GBM microenvironment and the mechanisms by which it is changed by the cross-talk between the glioma cells and the non-neoplastic cells in the TME, mainly the astrocytes, are not well understood.

The present inventor found that GBM modulates the metabolic activity of the TAAs and, using genome-scale metabolic network analysis, identified significant perturbations in several key metabolic pathways that might be associated with tumour progression. These include cholesterol metabolism, bile acid (synthesized from cholesterol) biosynthesis and hyaluronan metabolism, which was shown to maintain the glioblastoma stem-like cell tumourigenicity potential (FIG. 2F and Table 2). Given the unique role of astrocytes in controlling brain cholesterol homeostasis, the present inventor focused on studying the contribution of astrocyte-derived cholesterol to glioma pathogenicity. The results indicate that glioma cells rely on exogenous cholesterol to maintain their energy metabolism and support viability. Accordingly, deprivation of cholesterol carrying lipoproteins from their environment lowers the OXPHOS potential, which is important for tumour progression. Indeed, a variety of OXPHOS-targeting drugs, including metformin, Gboxin and IACS-010759, were shown to inhibit glioma proliferation and induce cell death. In this context, metformin combined with temozolomide (standard-of-care chemotherapy for GBM patients) or radiotherapy is currently in clinical trials for glioma (NCT02780024, NCT03243851). In addition, the present inventor demonstrates that both mouse and human glioma cells are dependent on cholesterol efflux from the astrocytes (FIGS. 5A-N, 11A-K and 12A-F), and that blocking the efflux of cholesterol from the astrocytes, by targeting astrocytic expression of the cholesterol transporter ABCA1, causes regression of tumour growth and prolongs mouse survival (FIGS. 6A-O). The physiological relevance of these findings to human disease is provided by scRNAseq data analysis of the TME of GBM patients, as well as TCGA data demonstrating an increased expression of astrocytic ABCA1 in the GBM TME and an inverse correlation with patient survival. Further support is provided by recent reports by Villa et al. (Cancer Cell 2016, 30: 683-693) that targeting the ABCA1-LDLR axis, using a brainpenetrant LXR agonist, kills glioma cells and attenuates tumour pathogenicity in a GBM xenograft model. The data demonstrate a tissue context in which reactive astrocytes in the TME supply the metabolic requirements of tumour cells, thereby exposing vulnerabilities that merit clinical exploitation and contributing to the recently emerging line of research that demonstrates a profound astrocyte-mediated metabolic dependent metabolic dependent regulation of neuro-pathologies. Notably, here the present inventor only addressed the cholesterol node in the astrocyte-tumour cell axis within the vast complexities of the metabolic network that governs GBM pathogenicity.

Astrocyte's diverse functions in health and disease encompass a continuum of cellular states with the potential for plasticity and reprogramming. Recent scRNAseq studies had provided valuable insights into basal and reactive-astrocyte heterogeneity in neuroinflammation and neurodegeneration. However, little is known about astrocyte heterogeneity in the GBM TME, and the underlying mechanisms driving it. To address the question of astrocyte diversity, the present inventor analysed the astrocyte scRNAseq data from the GBM patients by means of differential expression, pathway analysis and transcription factors enrichment. The present inventor found that the astrocytes clustered into two main populations (FIGS. 8J-Q and Table 4). Cluster A (Blue, 599 cells), which is enriched with the immune response transcripts, and cluster B (Pink, 453 cells), enriched in genes associated with cholesterol synthesis and bioenergetics (FIG. 8J-N). Interestingly, each cluster was also significantly (FDR <0.001) enriched by a unique transcription factor signature. The transcripts encoding AHR, ATF3, SOCS3, PITX1, RELB and CRBPB were mainly associated with cluster A, while the transcripts encoding NFIA, HES6, HES1, SOX11, SOX15, TFDP2 and PPARGC1A were mainly associated with cluster B (FIG. 8O). Notably, several transcripts encoding key transcription factors such as STAT1, STAT3, NFATC3, SOX9 and IRF1 displayed a pan-astrocyte activation pattern and were evenly expressed between the two clusters (FIG. 8O). Suggesting a heterogeneity in the astrocyte response to the GBM TME. To study if the difference in astrocyte cell density may be related to the differential expression in cholesterol synthesis between the two clusters, the present inventor analyzed the expression of fibroblast growth factor 2 (FGF2), whose expression is inhibited in high cell density in human astrocytes. The present inventor found that FGF2 expression was strongly associated with cluster A, and had no significant correlation with any of the key cholesterol synthesis transcripts that were shown to be associated with astrocyte density (FIGS. 8P and 8Q, respectively). Suggesting that astrocyte cell density plays a role in TAAs responses in the TME.

Without being bound by any theory, the present inventor has identified a mechanism by which astrocytes participate in the control of TAM immunity and sustain the metabolic landscape necessary for tumour survival. These findings shed new light on the pivotal role astrocytes play in promoting glioblastoma pathogenicity and the potential vulnerabilities caused by the tumour dependence on astrocyte immunometabolic support of the TME, thereby identifying candidate targets for therapeutic intervention.

TABLE 3
Sequences used for generating ABCA1 RNAi vectors

| SEQ | | |
|---|---|---|
| ID | | |
| NO | Name | Sequence (5′→3′) |
| 1 | shNT (*) | GAGTGCCACTTTCCGAATAAA |
| 2 | shAbca1 | GCGCGATAGCGCTAATAATTT |
| 3 | gfaABC1D | GATCTAACATATCCTGGTGTGGAGTAGGGGACGCTGCTCTGACAG |
| | | AGGCTCGGGGGCCTGAGCTGGCTCTGTGAGCTGGGGAGGAGGCA |
| | | GACAGCCAGGCCTTGTCTGCAAGCAGACCTGGCAGCATTGGGCTG |
| | | GCCGCCCCCCAGGGCCTCCTCTTCATGCCCAGTGAATGACTCACCT |
| | | TGGCACAGACACAATGTTCGGGGTGGGCACAGTGCCTGCTTCCCG |
| | | CCGCACCCCAGCCCCCCTCAAATGCCTTCCGAGAAGCCCATTGAG |
| | | CAGGGGGCTTGCATTGCACCCCAGCCTGACAGCCTGGCATCTTGG |
| | | GATAAAAGCAGCACAGCCCCCTAGGGGCTGCCCTTGCTGTGTGGC |
| | | GCCACCGGCGGTGGAGAACAAGGCTCTATTCAGCCTGTGCCCAGG |
| | | AAAGGGGATCAGGGGATGCCCAGGCATGGACAGTGGGTGGCAGG |
| | | GGGGGAGAGGAGGGCTGTCTGCTTCCCAGAAGTCCAAGGACACA |
| | | AATGGGTGAGGGGAGAGCTCTCCCCATAGCTGGGCTGCGGCCCAA |
| | | CCCCACCCCCTCAGGCTATGCCAGGGGGTGTTGCCAGGGGCACCC |
| | | GGGCATCGCCAGTCTAGCCCACTCCTTCATAAAGCCCTCGCATCC |
| | | CAGGAGCGAGCAGAGCCAGAGCAGGTTGGAGAGGAGACGCATCA |
| | | CCTCCGCTGCTCGCA |
| 4 | sgLuc2 | CACCGTTGGCGCTCAACTTTTACGA |
| | (**) | |
| 5 | SgAbca1 | CACCGGAGAGTCACTCACCCGGACA |
| | (#1) | |
| 6 | sgAbca1 | CACCGTTGGCGCTCAACTTTTACGA |
| | (#2) | |
| 7 | sgRNA | TACGTGACGTAGAAAGTA |
| | sequencing | |
| | primer | |

Table 3: “*” = Mayo L, Trauger S.A., Blain M., et al. Regulation of astrocyte activation by glycolipids drives chronic CNS inflammation. Nat Med. October 2014; 20(10): 1147-56. doi:10.1038/nm.3681; “**” = Hart T., Chandrashekhar M., Aregger M., et al. High-Resolution CRISPR Screens Reveal Fitness Genes and Genotype-Specific Cancer Liabilities. Cell. Dec. 3 2015; 163(6): 1515-26. doi:10.1016/j.cell.2015.11.015.

TABLE 4
Functional enrichment analysis
of human tumor-associated astrocytes

| ID | Term ID | Term Name | ES | FDR |
|---|---|---|---|---|
| 1 | R-HSA- | Defective ST3GAL3 causes | 0.98662 | 0.03965 |
| | 3656243 | MCT12 and EIEE15 | | |
| 2 | R-HSA- | Cholesterol biosynthesis | 0.64482 | 0.03965 |
| | 191273 | | | |
| 3 | R-HSA- | Glyoxylate metabolism and | 0.63704 | 0.03965 |
| | 389661 | glycine degradation | | |
| 4 | R-HSA- | Complex I biogenesis | 0.55068 | 0.03965 |
| | 6799198 | | | |
| 5 | R-HSA- | Respiratory electron transport, | 0.5376 | 0.03965 |
| | 163200 | ATP synthesis by chemiosmotic | | |
| | | coupling, and heat production | | |
| | | by uncoupling proteins. | | |
| 6 | R-HSA- | Respiratory electron transport | 0.5357 | 0.03965 |
| | 611105 | | | |
| 7 | R-HSA- | Regulation of cholesterol | 0.52005 | 0.03965 |
| | 1655829 | biosynthesis by SREBP (SREBF) | | |
| 8 | R-HSA- | Activation of gene expression | 0.51517 | 0.03965 |
| | 2426168 | by SREBF (SREBP) | | |
| 9 | R-HSA- | The citric acid (TCA) cycle and | 0.43726 | 0.03965 |
| | 1428517 | respiratory electron transport | | |
| 10 | R-HSA- | Innate Immune System | −0.30387 | 0.03965 |
| | 168249 | | | |
| 11 | R-HSA- | Signaling by Receptor | −0.33193 | 0.03965 |
| | 9006934 | Tyrosine Kinases | | |
| 12 | R-HSA- | Signaling by GPCR | −0.33459 | 0.03965 |
| | 372790 | | | |
| 13 | R-HSA- | Metabolism of vitamins | −0.38108 | 0.03965 |
| | 196854 | and cofactors | | |
| 14 | R-HSA- | SLC-mediated transmembrane | −0.39824 | 0.03965 |
| | 425407 | transport | | |
| 15 | R-HSA- | Cytokine Signaling in | −0.39854 | 0.03965 |
| | 1280215 | Immune system | | |
| 16 | R-HSA- | Interferon Signaling | −0.42312 | 0.03965 |
| | 913531 | | | |
| 17 | R-HSA- | L1CAM interactions | −0.42924 | 0.03965 |
| | 373760 | | | |
| 18 | R-HSA- | Signaling by Interleukins | −0.44046 | 0.03965 |
| | 449147 | | | |
| 19 | R-HSA- | GPCR ligand binding | −0.45853 | 0.03965 |
| | 500792 | | | |
| 20 | R-HSA- | Semaphorin interactions | −0.46668 | 0.03965 |
| | 373755 | | | |
| 21 | R-HSA- | Signaling by MET | −0.51052 | 0.03965 |
| | 6806834 | | | |
| 22 | R-HSA- | Response to elevated platelet | −0.53623 | 0.03965 |
| | 76005 | cytosolic Ca2+ | | |
| 23 | R-HSA- | Interferon alpha/beta signaling | −0.53748 | 0.03965 |
| | 909733 | | | |
| 24 | R-HSA- | ECM proteoglycans | −0.58288 | 0.03965 |
| | 3000178 | | | |
| 25 | R-HSA- | Non-integrin membrane-ECM | −0.60099 | 0.03965 |
| | 3000171 | interactions | | |
| 26 | R-HSA- | Cell surface interactions at | −0.60219 | 0.03965 |
| | 202733 | the vascular wall | | |
| 27 | R-HSA- | Class A/1 (Rhodopsin-like | −0.61315 | 0.03965 |
| | 373076 | receptors) | | |
| 28 | R-HSA- | Elastic fibre formation | −0.62358 | 0.03965 |
| | 1566948 | | | |
| 29 | R-HSA- | Extracellular matrix organization | −0.62407 | 0.03965 |
| | 1474244 | | | |
| 30 | R-HSA- | Peptide ligand-binding receptors | −0.63294 | 0.03965 |
| | 375276 | | | |
| 31 | R-HSA- | Interleukin-4 and Interleukin-13 | −0.63426 | 0.03965 |
| | 6785807 | signaling | | |
| 32 | R-HSA- | Sema4D in semaphorin signaling | −0.63503 | 0.03965 |
| | 400685 | | | |
| 33 | R-HSA- | Regulation of Insulin-like Growth | −0.64225 | 0.03965 |
| | 381426 | Factor (IGF) transport and | | |
| | | uptake by Insulin-like | | |
| | | Growth Factor Binding | | |
| | | Proteins (IGFBPs) | | |
| 34 | R-HSA- | Diseases of programmed | −0.65146 | 0.03965 |
| | 9645723 | cell death | | |
| 35 | R-HSA- | MET promotes cell motility | −0.65289 | 0.03965 |
| | 8875878 | | | |
| 36 | R-HSA- | Collagen degradation | −0.65325 | 0.03965 |
| | 1442490 | | | |
| 37 | R-HSA- | Interleukin-6 family signaling | −0.65494 | 0.03965 |
| | 6783589 | | | |
| 38 | R-HSA- | Interferon gamma signaling | −0.66473 | 0.03965 |
| | 877300 | | | |
| 39 | R-HSA- | Platelet Aggregation | −0.6806 | 0.03965 |
| | 76009 | (Plug Formation) | | |
| 40 | R-HSA- | Degradation of the | −0.68368 | 0.03965 |
| | 1474228 | extracellular matrix | | |
| 41 | R-HSA- | Other interleukin signaling | −0.68368 | 0.03965 |
| | 449836 | | | |
| 42 | R-HSA- | Basigin interactions | −0.68665 | 0.03965 |
| | 210991 | | | |
| 43 | R-HSA- | Syndecan interactions | −0.69992 | 0.03965 |
| | 3000170 | | | |
| 44 | R-HSA- | Laminin interactions | −0.71571 | 0.03965 |
| | 3000157 | | | |
| 45 | R-HSA- | MET activates PTK2 signaling | −0.7177 | 0.03965 |
| | 8874081 | | | |
| 46 | R-HSA- | Collagen biosynthesis and | −0.71836 | 0.03965 |
| | 1650814 | modifying enzymes | | |
| 47 | R-HSA- | Collagen chain trimerization | −0.73086 | 0.03965 |
| | 8948216 | | | |
| 48 | R-HSA- | Immunoregulatory interactions | −0.73656 | 0.03965 |
| | 198933 | between a Lymphoid | | |
| | | and a non-Lymphoid cell | | |
| 49 | R-HSA- | Collagen formation | −0.73969 | 0.03965 |
| | 1474290 | | | |
| 50 | R-HSA- | TNFR1-induced proapoptotic | −0.74918 | 0.03965 |
| | 5357786 | signaling | | |
| 51 | R-HSA- | Cell-extracellular matrix | −0.77086 | 0.03965 |
| | 446353 | interactions | | |
| 52 | R-HSA- | Integrin cell surface interactions | −0.77262 | 0.03965 |
| | 216083 | | | |
| 53 | R-HSA- | Nicotinate metabolism | −0.77888 | 0.03965 |
| | 196807 | | | |
| 54 | R-HSA- | Assembly of collagen fibrils | −0.79098 | 0.03965 |
| | 2022090 | and other multimeric | | |
| | | structures | | |
| 55 | R-HSA- | Nicotinamide salvaging | −0.8154 | 0.03965 |
| | 197264 | | | |
| 56 | R-HSA- | Interleukin-10 signaling | −0.885 | 0.03965 |
| | 6783783 | | | |
| 57 | R-HSA- | Chemokine receptors | −0.91066 | 0.03965 |
| | 380108 | bind chemokines | | |
| 58 | R-HSA- | Crosslinking of collagen fibrils | −0.93 | 0.03965 |
| | 2243919 | | | |
| 59 | R-HSA- | Alternative complement | −0.9999 | 0.03965 |
| | 173736 | activation | | |
| 60 | R-HSA- | Metal sequestration by | −1 | 0.03965 |
| | 6799990 | antimicrobial proteins | | |

Additional abbreviations:
RPL22 = Ribosomal protein L22;
TMZ = Temozolomide;

### Example 7

### Targeting of Cholesterol Efflux to Tumor Cells Using FDA-Approved Drugs

The present inventor has evaluated the antitumor potency of FDA-approved cholesterol-lowering drugs to decrease astrocyte-driven cholesterol efflux to tumor cells, to halt tumor progression in-vitro and in-vivo using GBM-animal models, and to reprogram the tumor chemoresistance to therapy (TMZ) against primary and postoperative GBM recurrence.

Without being bound by any theory, the present inventor has hypothesized that drug-mediated inhibition of cholesterol synthesis, and inhibition of ABCA1 activity, would limit cholesterol efflux to the tumor cells, depriving the cells of vital energy sources, lowering their apoptotic threshold, and thus should attenuate tumor survival.

The following drugs were tested:

Inhibitors of cholesterol synthesis: The BBB-permeable statins inhibit cholesterol synthesis by inhibiting 3-hydroxy-3-methylglutaryl-CoA reductase (HMGCR), a rate-limiting enzyme in the cholesterol synthesis pathway. These include, but are not limited to, Simvastatin, Lovastatin, and Pitavastatin.

Inhibitors of cholesterol transporter: Probucol (Lorelco) and/or glyburide (Glibenclamide) inhibit the activity of the cholesterol transporter ABCA1.

Probucol is a strong, BBB-permeable, ABCA1 inhibitor that was also shown to prevent BBB dysfunction and mitigate cognitive and hippocampal synaptic impairments in Alzheimer's models.

Glyburide is also a potent ABCA1 inhibitor; however, it doesn't cross well the undisrupted BBB. Glyburide can also inhibit Sulfonylurea receptor 1 (SUR1), a known component of ATP-sensitive potassium channels, and thus serves to significantly reduce edema and brain swelling in different brain injuries ranging from stroke to metastatic brain tumor, which is one the main mode-of-actions of Bevacizumab (Avastin), a recently FDA-approved (2009) for the treatment of recurrent or progressive GBM. Experimental design

To ensure statistical power, for in vitro assays, three to five replicates within each condition were used to represent intragroup variations sufficiently and allow for defining statistical significance. In all in vivo experiments, 8-10-week-old mice are used, and six to ten mice are included within each group to ensure statistical power, enabling to distinguish tumor burden and survival rates across groups statistically. Before treatment, mice are randomized based on bioluminescent imaging to ensure similar average tumor sizes across groups. Animal experiments and IHC analysis are performed blinded when possible. Statistical analyses are performed using PRISM GraphPad.

### In-Vitro Assays for Determining the Potential of the FDA-Approved Drugs to Target Cholesterol Efflux to the Tumor Cells:

(i) Accumulation of cholesterol in tumor cells—Mouse and human GBM cells (GL261 and C2-TA, or U87EGFRvIII and GBM39, respectively) are cultured in the presence (co-culture) or absence (mono-culture) of astrocytes under lipoprotein deprivation (in media-supplemented with lipoprotein-deprived serum (LPDS)), with different concentrations of simvastatin, lovastatin, pitavastatin, probucol, glyburide (hence, cholesterol-lowering drugs), or vehicle. After 24 hours-48 hours the cholesterol accumulation in the tumor cells is assessed using Filipin-III staining or by using commercial kits (e.g., Amplex Red Cholesterol Assay Kit, Invitrogen). Of note, to validate that the observed effects of probucol and glyburide are indeed mediated by ABCA1, the same experimental design is repeated with astrocytes in which the expression of ABCA1 is targeted. To this end, the present inventor uses the lentivirus-based system optimized for astrocyte-specific knock-down as described hereinabove, and the ability of the different drugs to attenuate the efflux of astrocyte-derived cholesterol to the glioma cells is tested. In this system, the truncated GFAP promoter, GfaABC1D, drives the expression of a miR30-based shRNA or CRISPR and a GFP reporter, targeting Abca1 or control seq (not targeting sequence for the RNAi system or luciferase gene Luc2 in the CRISPR-Cas9 systems (FIGS. 6A-O and FIG. 8C).

(ii) Viability of glioma cells following the use of cholesterol-lowering drugs—To test whether drug-mediated inhibition of cholesterol efflux rescues the glioblastoma cells from cholesterol depredation-induced death, mouse and human GBM cells (GL261 and C2-TA, or U87EGFRvIII and GBM39, respectively) are cultured in the presence (co-culture) or absence (mono-culture) of astrocytes and subjected to lipoprotein deprivation, with optimal concentrations of simvastatin, lovastatin, pitavastatin (0-40 μM), probucol, glyburide (0-100 μM), or vehicle, and the glioma viability is determined by Annexin-V assay. Moreover, statins and probucol, or glyburide, target different nodes in the cholesterol efflux mechanism (synthesis and extracellular export). Therefore, the present inventor further tests whether the combinations of the two drug groups would have a synergistic effect on glioma viability.

To validate the dependency on astrocyte-driven cholesterol, the experiments is repeated and exogenous cholesterol is added, to test whether cholesterol can rescue the cells from the effect of the drugs. Of note, both blocking of cholesterol synthesis in the reactive astrocytes by the statins (using lovastatin, Pitavastatin or Simvastatin) or cholesterol accumulation following the inhibition of ABCA1 activity, may be detrimental to the astrocytes, leading to their death (which is a desired effect, based on the models using astrocyte depletion in GBM-bearing mice), therefore the present inventor monitors the astrocyte viability using the Annexin-V assay (measures GBM cell death in this case).

Indeed, the present inventor found that treatment of cholesterol-deprived astrocyte-glioma co-cultures with cholesterol-lowering drugs attenuated the astrocyte-dependent rescue of the glioma cells from cholesterol deprivation, with different efficiencies (FIGS. 15A-D). Glyburide was the most potent, followed by probucol, simvastatin, and pitavastatin, while lovastatin showed a minimal effect.

These results demonstrate the efficacy of using cholesterol lowering drugs for treating glioblastoma by blocking efflux of cholesterol from astrocytes in the TME to the cancerous GBM cells.

### Example 8

Cholesterol-Lowering Drugs have the Ability to Halt Gbm Pathogenicity

As shown in Example 6 of the Examples section above, the efflux of cholesterol from tumor-associated astrocytes to the tumor cells was blocked in-vivo by targeting astrocytic expression of the cholesterol transporter ABCA1. Thus, astrocyte-specific shRNA lentiviruses, cause a reduction in the cholesterol content of the GBM cells, regression of tumor growth and prolongs mouse survival (FIGS. 6A-O and 8C). The physiological relevance of these findings to human disease is provided by scRNAseq data analysis of the TME of GBM patients, as well as TCGA data demonstrating an increased expression of astrocytic ABCA1 in the GBM TME and an inverse correlation with patient survival (FIGS. 6A-O). Further support is provided by recent reports by Villa et al. 2016 (Cancer Cell 30, 683-693) that targeting the ABCA1-LDLR axis, using a brain-penetrant LXR agonist, kills glioma cells and attenuates tumor pathogenicity in a GBM xenograft model. FIGS. 15A-D shows that cholesterol-lowering drugs significantly inhibit the astrocyte-cholesterol-tumor cell axis. Thus, the present inventor has reasoned that cholesterol-lowering drugs would limit the astrocyte-derived cholesterol available to the tumor cells and thus mitigate the astrocyte metabolic support of the tumor, halting GBM progression. To investigate this hypothesis, syngeneic immunocompetent murine models of GBM are used, in which the mice are intracranially implanted with tumor cells that recapitulate many of the features of human GVM. The use of this GBM model allows investigation of the drug's response in the context of an intact immune system in the TME; the cross-talk between astrocyte and the tumor-associated macrophages (TAMs), and that cholesterol regulates TAMs polarization and induces CD8⁺ T Cell exhaustion in peripheral tumors.

### Experimental Design

To investigate this hypothesis, C57Bl/6 mice are implanted with GFP⁺ luciferase-expressing glioma cells (GL261 or CT-2A), using two subtype models—primary or recurrent GBM/brain tumor resection (as illustrated in FIG. 16A), and are treated daily with probucol, glyburide, or vehicle control, with or without a potent statin (e.g., simvastatin as shown in FIG. 15A). The dosage of the different drugs is optimized based on FDA guidelines for conversions from human-accepted dosages (Table 5 below).

TABLE 5
Dosage of different FDA-approved drugs for in-vivo experiments

| | Human | Eq. mouse | Tested Dosage |
|---|---|---|---|
| | (max dosage) | dosage | (mg/kg/day) |
| Probucol | 600 mg/day | 124 mg/kg/day | 30, 60, 90, 120 |
| Glyburide | 20 mg/day | 4 mg/kg/day | 1, 2, 3, 4 |
| Statins | 80 mg/day | 16 mg/kg/day | 4, 8, 12, 16 |

In the primary GBM model (pGBM)—10 days after tumor implantation, the mice are treated with cholesterol-lowering drugs (FIG. 16A). In the recurrent GBM model (rGBM), 12-14 days after inoculation of the mice with the tumor cells, the visible tumor(s) are surgically removed, based on GFP expression, under a stereoscope. The mice are then allowed to recover for a week and then are treated with cholesterol-lowering drugs (FIG. 16A). Of note, tumor formation or recurrence is monitored by bioluminescence (BLI) signals from the tumor cells on in-vivo imaging system (IVIS) spectrum and quantified with Living Image software. Mice are monitored for tumor growth, peritumoral brain edema formation, and mouse survival until the experimental endpoint (extreme lethargy or body weight reduction of greater than 20%), or are euthanized seven days after treatment with the cholesterol-lowering drugs to assess tumor burden, angiogenesis, BBB stability, and immunomodulatory activity in-situ. Tumor volume is evaluated in-vivo by determining tumor BLI, and by histopathology analysis postmortem. Peritumoral brain edema is evaluated in-vivo using IVISense™ Edema (Superhance™ 680) fluorescent probe. Tumor pathology is further analyzed by studying cell infiltration to the CNS (H&E, CD3, IBA1, and CD45 staining), BBB integrity (Fibrin staining), astrocyte activation (GFAP, ALDH1L1, and AQP4 staining), tumor proliferation (Ki67 expression), angiogenesis (CD31 staining), cellular viability (cleaved caspase-3 staining and TUNEL staining), and evaluation of tumor insaneness and tumor necrosis. Immunomodulatory activity TAMs and CD8⁺ T-cells is analyzed by FACS-sorting the cells and subjecting them to transcriptomic analysis [qRT-PCR (Real-Time Quantitative Reverse Transcription PCR) or NanoString]. The expression of TAMs genes associated with glioma invasion (e.g., Mmp2, Mmp9, Mmp14, Cx3Cl1, Csf1, and Egf), angiogenesis (e.g., Vegfa, Fgf2, and Il6), immune suppression [e.g., Il10, Tgfb, Cd274 (PD-L1), Cd95l], tumor proliferation (e.g., Egf, Il1b, 116, and Gdnj) is determined, and evaluation of the TAM differentiation is determined using the expression of both M1-like (Cd80, Cd86, Stat1, Cd274, Il1b, Tnfa, and Il27), M2-like (e.g., Il10, Cd206, Cd163, Cd204, Arg1, Vegfa, Klf4, and Pparg), and GBM-related [Csf1, csf1r, Ahr, Entpd1, Tlr2, Hif1, I123a, Cxcr4, cxcl12) markers.

CD8⁺ T-cells are assayed for the expression of cytotoxic genes (e.g., Gzmb, Tnf, Ifng, Fasl) and as cholesterol was shown to induce CD8⁺ T Cell Exhaustion in the TME, also for known exhaustion markers (PD1, 2B4, TIM-3, and LAG-3).

In addition, GFP⁺ tumor cells are FACS-sorted (17-20 days after initial implantation) and the changes to the tumor phenotype are characterized by measuring the following properties:
(1) immunogenic properties (e.g., MHC-I, PD-L1) and expression of immunosuppressive cytokines such as TGFβ, IL-10, and PGE2; by qRT-PCR and FACS analyses;
2) expression of factors known to be associated with angiogenesis (e.g., Vegf, Cd93, Hif1a; by qRT-PCR);
3) expression of cytokines and chemokines that may affect the tumor microenvironment (e.g., Ccl2, Sdf1, Csf1, Csf3, Mic1; by qRT);
4) genes known to contribute to tumor progression and invasion including Idol, Mmp2/3/9/14, Stat3, Cxcr4, Egf, and glutamate transporters; by qRT-PCR and if relevant also by FACS or WB (Western Blot)—e.g., to determine STAT3 phosphorylation).

Finally, cholesterol content in GBM cells, astrocytes, TAMs, and CD8⁺ T-cells is determined by Filipin III staining or by Mass spectrometry analysis to validate the efficiency of the cholesterol-lowering drugs.

### Example 9

### Analysis of Cognitive Phenotype on GBM-Mice Following Treatment with Cholesterol-Lowering Drugs

Cholesterol is ubiquitous in the CNS and is essential for normal brain function, including signaling and synaptic plasticity. Brain cholesterol metabolic deficiency has been linked to various neurological disorders, such as Alzheimer's disease, Parkinson's disease, and Huntington's disease.

To analyze the cognitive phenotype of the GBM-bearing mice, treated with the different cholesterol-lowering drug regimes, compared to vehicle control, the mice's cognitive function is analyzed by Novel object recognition, open field test, and elevated plus maze, which were shown to be affected by cholesterol levels and associated with GBM (Feng, X., et al., 2018. Elife 7, e38865).

The analysis involves using in-vitro studies (as described above), in-vivo tumor implantation (as described above), craniotomy (as described in Benbenishty, A. et al. Plos Biol 17, e2006859 (2019); which is fully incorporated herein by reference), and in-vivo imaging (as described hereinabove and in Levy, A. et al. Neuro-oncology 14, 1037-49 (2012); which is fully incorporated herein by reference).

Tumor restriction is performed based on the Cells' GFP expression, or by intravenous injection of fluorescein at the beginning of the procedure to better visualize the tumor, as described in Reste, P. J. L. et al. Cancer Lett 494, 73-83 (2020); which is fully incorporated herein by reference).

Evaluating of tumor volume and peritumoral brain edema can be also achieved using T2 weighted MRI imaging (as described in Levy, A. et al. 2012, supra).

Thus, these studies identify the optimal treatment regime for primary and recurrent GBM, determined by elevating the tumor burthen, prolonging mice survival, and demonstrating minimal cognitive decline.

As shown in FIGS. 16A-E, a continuous treatment with probucol, at a quarter of the human equivalent dose (HED), significantly attenuated tumor growth and weight loss associated with tumor burden and improved the mice's survival (FIGS. 16B-D). Notably, probucol and glyburide differ in their potency and ability to cross the BBB, and predict that they would have different efficiencies associated with the tumor stage (FIG. 16E).

### Example 10

### Cholesterol-Lowering Drugs have a Capacity to Increase the Apoptotic Sensitivity of Glioblastoma

### Experimental Results

(i) Depriving glioma cells of exogenous cholesterol significantly sensitized the cells to apoptotic stimuli (FIGS. 17A-C), suggesting that regulating exogenous cholesterol controls the glioma cell apoptotic threshold.

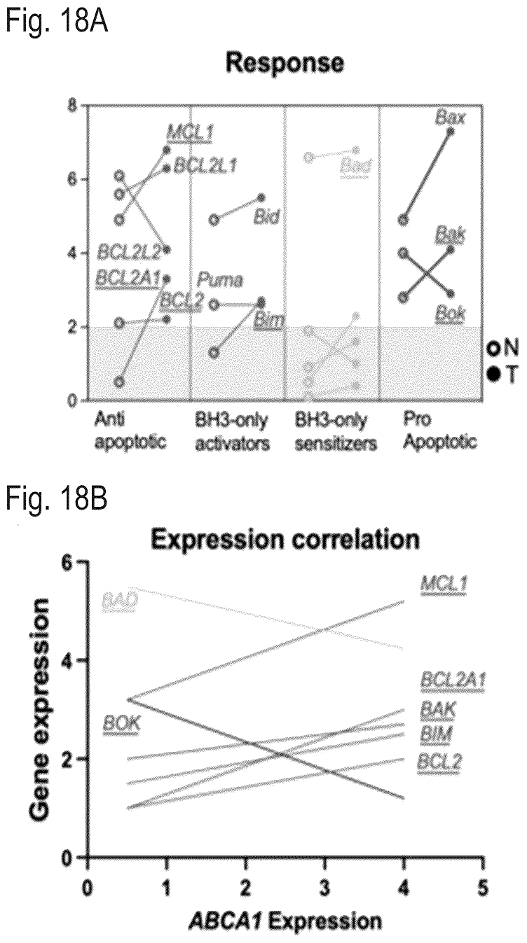

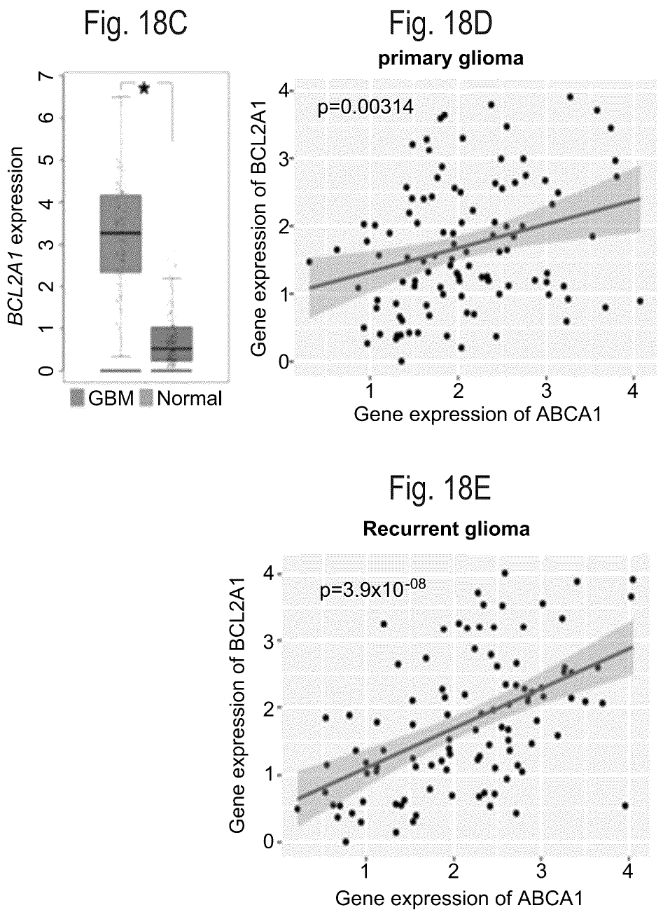

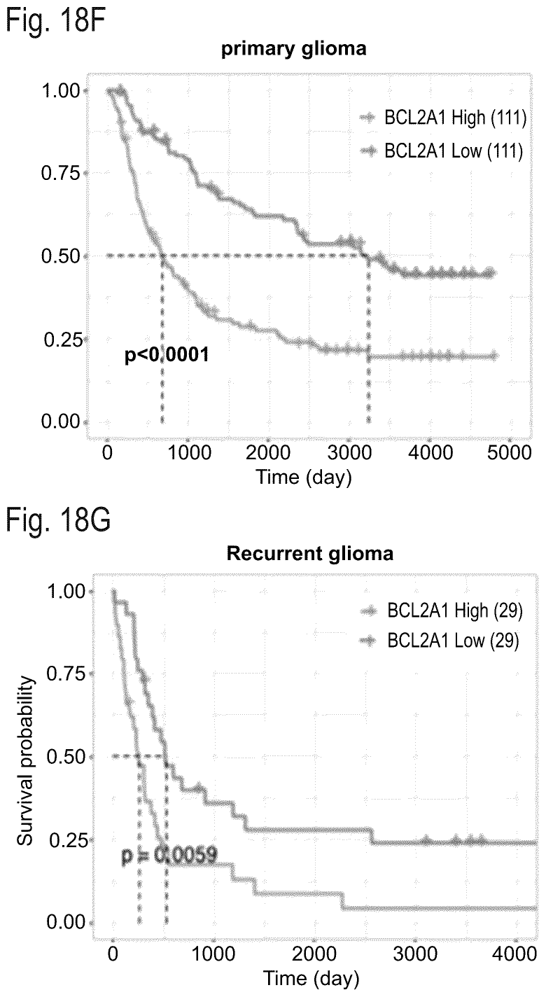

(i) Analysis of TCGA data not only identified the regulation of BCL-2 family genes but also found a significant correlation between ABCA1 expression levels and the expression of BCL-2 family genes (FIGS. 18A-B). For example, the expression of the anti-apoptotic BCL2A1 (BFL1), which is known to support resistance to chemotherapeutic drugs, is upregulated in GBM and demonstrates a significant positive correlation with ABCA1 expression, and its expression is associated with decreased survival of glioblastoma patients (FIGS. 18C-E). Thus, suggesting that cholesterol efflux in the GBM TME regulates the expression of pro and anti-apoptotic BCL-2 family genes.

Without being bound by any theory, the present inventor has hypothesized that cholesterol in gliomas plays an important role in resistance to apoptosis, and depriving gliomas of cholesterol can increase apoptotic sensitivity.

Determining the role of cholesterol in regulating the apoptotic threshold of glioblastoma cells—To determine whether cholesterol levels, specifically cholesterol levels in the mitochondria, control “mitochondrial priming” of glioblastoma cells, cholesterol levels of mouse and human glioblastoma cells are perturbated and the effect on the cell's apoptotic threshold is determined in non-stimulated cells (control) or mitochondria-dependent apoptotic stimuli (e.g., staurosporine, doxorubicin, or TMZ. To reduce global cholesterol levels, exogenous cholesterol is depleted by culturing the cells in lipoprotein-deficient serum (LPDS), or cholesterol is extracted using 2-hydroxypropyl-β-cyclodextrin (HMPCD). Whereas to promote the accumulation of cholesterol in the mitochondria, the cells are treated with U18666A, which was previously shown to promote the accumulation of cholesterol in the mitochondrial membranes, reduce Bax activation (oligomerization) in the mitochondria, and inhibit mitochondria-dependent apoptotic stimuli. Of note, the specificity of the U18666A response is further validated by co-treating the cells with U18666A+HMPCD, which should ameliorate U18666A-dependent cholesterol accumulation in the mitochondria, and by overexpression or knockdown of STARD3 (StAR Related Lipid Transfer Domain Containing 3; also known as MLN64) the main endosomal cholesterol transporter responsible for the trafficking of exogenous cholesterol to the mitochondria. Cholesterol perturbation is validated by Mass spectrometry analysis of whole cells or isolated mitochondria or by Filipin staining (mitochondrial cholesterol is evaluated by Cyotchorme C and Filipin co-staining for confocal microscopy analyses). Next, the expression of BCL-2 proteins (e.g., BCL2, BCL2A1, MCL1, BIM_{EL/L}, BAD) and activation (Bid/tBid, Bak, and Bax oligomerization) are determined by western blot, the cell apoptotic threshold is determined using BH3 profiling (a functional assay which measures the cumulative interaction and dependencies of these proteins), and cell viability is determined using Annexin-V or LDH-Assay. Collectively, these studies provide a comprehensive portrait of cholesterol's role in the anti-apoptotic machinery responsible for resistance to cell death in one of the most refractory of human tumors.

Testing the capacity of cholesterol-lowering drugs to increase the apoptotic sensitivity of glioblastoma—Mouse and human glioma cells are pre-treated with the cholesterol-lowering drugs, and subjected to TMZ treatment or left untreated, and the cell apoptotic threshold and survival is evaluated as detailed above.

Without being bound to any theory, the present inventor has uncovered that cholesterol-lowering drugs would prime the cells, making them more susceptible to intrinsic apoptotic stimulation.

Indeed, as shown in FIGS. 19A-C, treating mouse GL261 glioma cells with cholesterol-lowering drugs enhanced the cell sensitivity to cytotoxic chemotherapy (TMZ) by 2-3 fold compared to internal control, an up to 8-fold compared to vehicle control (FIGS. 19A-C).

Testing the ability of the optimal regimes of the cholesterol-lowering drugs to change the clinical response to TMZ cytotoxicity of GBM-bearing mice and to halt tumor progression—To assess the feasibility of such an approach, as an initial experimental set up, the present inventor has restricted the treatment with the ABCA1 inhibitor probucol to ±2 days before the TMZ treatment course, to limit its effect to that of the regulation of cholesterol-mediated priming of the glioma cells (FIGS. 20A-C). The results show that even this limited course of treatment with Probucol was able to halt GBM pathogenicity, and dramatically enhanced the therapeutic effect of TMZ (FIGS. 20A-C). Importantly, both probucol and TMZ were administered far below the recommended HED for both drugs. Next, intermediate (as in FIGS. 20A-C) or continuous (as in FIGS. 16A-E) administration modes of cholesterol-lowering drugs, with different TMZ dosages (2.5, 7.5, 25, or 66.7 mg/kg, which is closer to the recommended HED for TMZ) in models of primary and recurrent GBM are studied. GBM pathogenicity (tumor burden, weight loss, and mice survival) and the mice's cognitive profile are analyzed to determine the optimal treatment course.

The present inventor further investigates how the optimal treatment regime of TMZ and the cholesterol-lowering drugs regulates 1) cholesterol levels in the TME, 2) the viability and activation profile of the cells in the TME, and 3) the apoptotic threshold of the glioma cells using the BH3 profiling and analysis of Bcl2 family protein expression and activation (by IHC, or by sort the GFP⁺ glioma cells, followed by qRT or western blot analyses).

### Example 11

### PCSK9 Overexpression in the Cancer Microenvironment Halts Glioma Progression

Astrocytes were transduced with a lentivirus comprising a coding sequence of wild type PCSK9 or with an empty lentivirus (which does not comprise the coding sequence of PCSK9) and 24 hours later, the media was collected and transferred to glioma cells.

As shown in FIG. 21B, secretion of PCSK9 into the culture medium (astrocyte-conditioned medium) resulted in decreased levels of LDLR in GL261 glioma cells which were treated with the astrocyte-conditioned media for 24 hours, demonstrating that PCSK9 can control the levels of LDLR in glioma cells.

PCSK9-encoding or empty lentiviruses were intracranially injected into the TME of GL261-bearing mice 9 and 15 days after tumor implantation, and the effect on tumor growth was determined based on tumor size as measured by bioluminescence. As shown in FIG. 21C, intracranial injection of PCSK9-encoding lentivirus attenuates GBM progressions.

In addition, the effect of PCSK9 over-expression on subjects having GBM was determined by assessing the overall survival of the treated mice. As shown in FIG. 21D, Kaplan-Meier curves showed prolonged survival in mice which expressed the exogenous PCSK9 by the lentivirus.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a brain tumor in a subject in need thereof, the method comprising administering to the subject a therapeutically effective amount of an agent capable of specifically downregulating activity or expression of a component of the lipid synthesis and/or transportation pathways in a reactive non-cancerous astrocyte in the tumor microenvironment, wherein the agent is specific to said reactive astrocyte in the tumor microenvironment and not to a cancerous cell of the brain tumor, thereby treating the brain tumor in said subject.

2. The method of claim 1, wherein said agent is conjugated directly or indirectly to a targeting moiety capable of binding said reactive non-cancerous astrocyte of the tumor microenvironment and not to a non-reactive astrocyte or a cancerous cell of the brain tumor.

3. The method of claim 1, wherein said lipid is selected from the group consisting of a cholesterol, a cholesteryl ester (CE), a triglyceride and a sphingolipid.

4. The method of claim 1, wherein said agent is an efflux inhibitor which inhibits release of a lipid from said reactive non-cancerous astrocyte in the tumor microenvironment, optionally, wherein said lipid is cholesterol, optionally, wherein said efflux inhibitor is an inhibitor of the ATP-binding cassette transporter A1 (ABCA1).

5. The method of claim 1, wherein said agent is a small molecule, optionally, wherein said small molecule is Probucol or Glyburide, or an analogue thereof, optionally, wherein said agent is a combination of Probucol and chemotherapy, and optionally, wherein said chemotherapy is temozolomide (TMZ).

6. The method of claim 1, wherein said component of said lipid synthesis or transportation pathway is a molecule associated with the de-novo cholesterol synthesis pathway, cholesterol catabolism to oxysterols, liver-X-receptors (LXRs), oxysterols catabolism, and/or with the induction of Mylip.

7. The method of claim 2, wherein said targeting moiety is an antibody, an aptamer, a peptide or a particle, optionally, wherein said antibody is a T cell receptor-like antibody.

8. The method of claim 5, wherein said small molecule is selected from the group consisting of a Probucol, Glyburide, a LDLR antisense/decoy molecule, a Menin inhibitor, a Statin, AY-9944, D-003, Avasimibe, Nystatin, Ezetimibe, Fenofibrate, 2-Hydroxypropyl-β-cyclodextrin, Omega-3-acid ethyl esters and an analogue thereof.

9. The method of claim 1, wherein said agent is a DNA editing molecule or an RNA silencing molecule.

10. The method of claim 1, wherein said agent is comprised in a nucleic acid construct under the transcriptional control of a cis acting regulatory element specifically active in said reactive non-cancerous astrocyte, optionally, wherein said cis acting regulatory element is an astrocyte-specific promoter, optionally, wherein said astrocyte-specific promoter is a glial fibrillary acidic protein (GFAP) promoter, optionally, wherein said glial fibrillary acidic protein (GFAP) promoter comprises SEQ ID NO: 3.

11. The method of claim 10, wherein said nucleic acid construct is encapsulated in a particle, optionally, wherein said particle is an Adeno-associated virus (AAV) particle.

12. The method of claim 7, wherein said antibody is capable of binding a reactive non-cancerous astrocyte MHC-I complex.

13. The method of claim 1, further comprising administering to the subject a therapeutically effective amount of a molecule which is associated with lipid uptake by the tumor cells or immune cells in the tumor microenvironment.

14. A method of treating a brain tumor in a subject in need thereof, the method comprising administering to the subject a therapeutically effective amount of a molecule which is associated with lipid uptake by the tumor cells or immune cells in the tumor microenvironment, or a polynucleotide encoding same, thereby treating the brain tumor in said subject, optionally, wherein said molecule or said polynucleotide is comprised in or associated with a particle suitable for delivery into a brain of the subject.

15. The method of claim 13, wherein said molecule which is associated with lipid uptake by the tumor cells or immune cells in the tumor microenvironment is Proprotein convertase subtilisin/kexin type 9 (PCSK9).

16. The method of claim 1, further comprising administering to the subject chemotherapy and/or radiation therapy.

17. The method of claim 1, wherein said brain tumor is Glioblastoma.

18. A chimeric polynucleotide comprising a nucleic acid sequence encoding an expression product capable of downregulating an activity or expression of a component of the lipid synthesis and/or transportation pathways, and another heterologous nucleic acid sequence comprising a cis acting regulatory element specifically active in a reactive non-cancerous astrocyte of the microenvironment of the tumor but not in a non-reactive astrocyte or a cancerous cell of a brain tumor, optionally, wherein said cis acting regulatory element is a promoter.

19. A composition of matter comprising the chimeric polynucleotide of claim 18, comprising a particle encapsulating said chimeric polynucleotide, optionally, wherein said particle is an Adeno-associated virus (AAV) particle.

20. An article of manufacture comprising a small molecule capable of downregulating an activity or expression of a component of the lipid synthesis and/or transportation pathways, said small molecule being conjugated to an antibody or fragment thereof capable of binding to a reactive non-cancerous astrocyte in the tumor microenvironment and not to a non-reactive astrocyte or a cancerous cell of the brain tumor, optionally, wherein said antibody is a T cell receptor-like antibody, optionally, wherein said antibody is capable of binding a reactive astrocyte MHC-I complex.

21. A monocyte expressing a heterologous PCSK9 mRNA or protein.
